(19) United States
(12) Patent Application Publication
(10) Pub. No.: US 2024/0288427 A1
(43) Pub. Date: Aug. 29, 2024

(54) ANTIBODY CAPABLE OF BINDING SPECIFICALLY TO NUCLEOCAPSID PROTEIN OF SEVERE ACUTE RESPIRATORY SYNDROME CORONAVIRUS-2 OR FRAGMENT THEREOF, AND USE OF SAID ANTIBODY OR SAID FRAGMENT

(71) Applicants: TOYOBO CO., LTD., Osaka (JP); NATIONAL UNIVERSITY CORPORATION UNIVERSITY OF TOYAMA, Toyama (JP)

(72) Inventors: Takashi KAWAHATA, Tsuruga (JP); Takahiro MASUYA, Tsuruga (JP); Kosuke YUHARA, Tsuruga (JP); Kengo NISHIMURA, Otsu (JP); Hiroaki KITAZAWA, Tsuruga (JP); Toshihiro KUROITA, Osaka (JP); Masaharu ISOBE, Toyama (JP); Nobuyuki KUROSAWA, Toyama (JP)

(73) Assignees: TOYOBO CO., LTD., Osaka (JP); NATIONAL UNIVERSITY CORPORATION UNIVERSITY OF TOYAMA, Toyama (JP)

(21) Appl. No.: 18/251,081

(22) PCT Filed: Oct. 28, 2021
(86) PCT No.: PCT/JP2021/039754

§ 371 Date: Apr. 28, 2023

(30) Foreign Application Priority Data
Oct. 30, 2020 (JP) 2020-183273

(51) Int. Cl. G01N 33/569; C07K 16/10

(52) U.S. Cl. CPC G01N 33/56983; C07K 16/1003; C07K 2317/34; G01N 2333/165; G01N 2469/10

(57) ABSTRACT

Provided are an antibody or fragment thereof specifically bindable to the NTD or CTD of the N protein of SARS-CoV-2, and use of the antibody or fragment thereof. An antibody or fragment thereof specifically bindable to the amino acid sequence of SEQ ID NO: 1 or 2 is disclosed.

### TECHNICAL FIELD

Techniques related to antibodies or fragments thereof specifically bindable to the nucleocapsid protein of severe acute respiratory syndrome coronavirus-2 (SARS-CoV-2), and use of the antibodies or fragments thereof are provided.

### BACKGROUND ART

SARS-CoV-2 is the causative virus of novel coronavirus infection (COVID-19), which has been rapidly spreading worldwide since early 2020. SARS-CoV-2 is composed of a nucleocapsid and an envelope surrounding the nucleocapsid, which is similar to typical coronaviruses. The nucleocapsid contains the viral genome (RNA) and the nucleocapsid protein (N protein) binding to the viral genome. The envelope contains a lipid and a spike protein (S protein), a membrane protein (M protein), and an envelope protein (E protein) that bind to the lipid.

The N protein is involved in viral core formation, as well as in packaging and transcription of the viral genome. The N protein has a structure in which the N-terminal domain (NTD) and C-terminal domain (CTD) are joined via a linker with a serine (S)/arginine (R)-rich region. NPL 1 discloses that the N protein is heavily phosphorylated, showing mapping of phosphorylated sites. Due to the conservation of the amino acid sequence of the N protein among strains, the N protein is usable as a diagnostic marker.

### CITATION LIST

### Non-Patent Literature

NPL 1: Klann et al., Molecular Cell, 80(1), 2020, pp. 164-174

### SUMMARY OF INVENTION

### Technical Problem

The inventors found that because the linker with an S/R-rich region (a large amount of phosphorylated sites) of the N protein of SARS-CoV-2 can be cleaved by apoptosis-inducing cysteine proteases (e.g., caspase 3 and caspase 6) or by autolytic action, even an antibody specifically bindable to the N protein of SARS-CoV-2 may not enable the detection of the N protein of SARS-CoV-2, depending on the site to which the antibody binds.

Thus, the primary object of the present invention is to provide an antibody or fragment thereof specifically bindable to the NTD or CTD of the N protein of SARS-CoV-2, and use of the antibody or fragment thereof.

The present inventors also found that in the method for detecting the N protein of SARS-CoV-2 by forming a sandwich structure with two different antibodies specifically bindable to the N protein, the detection sensitivity for the N protein of SARS-CoV-2 is decreased due to cleavage of the linker with an S/R-rich region hindering sandwich structure formation, for example, if two antibodies for use are an antibody specifically binding to the NTD and an antibody specifically bindable to the CTD.

Thus, the secondary object of the present invention is to provide a kit and a method for detecting the N protein of SARS-CoV-2 with high sensitivity.

### Solution to Problem

The present inventors conducted extensive research to achieve the objects and then found many kinds of antibodies and fragments thereof specifically bindable to the NTD or CTD of the N protein of SARS-CoV-2. The inventors also found that the use of two or more antibodies or fragments thereof that specifically bind to the NTD and that each recognize a different epitope in the NTD and/or two or more antibodies or fragments thereof that specifically bind to the CTD and that each recognize a different epitope in the CTD enables the detection of the N protein with high sensitivity. The inventors conducted further research based on these findings and completed the present invention.

The present invention includes the following aspects.

### Item 1.

An antibody or fragment thereof specifically bindable to the amino acid sequence of SEQ ID NO: 1 or 2 (antigen-binding fragment).

### Item 2.

An antibody or fragment thereof (antigen-binding fragment), comprising a heavy-chain CDR1 containing the amino acid sequence represented by the following formula (A-1) or (A-2):

GEK^{A31}X^{A41}X^{A51}X^{A61}X^{A71}X^{A81} (A-1)
wherein
X^{A31} represents T or S,
X^{A41} represents F, L, or I,
X^{A51} represents D, S, N, or T,
X^{A61} represents D, N, S, I, or T,
X^{A71} represents Y, F, or N,
X^{A81} represents G, W, Y, S, or T, and

GFX^{A33}X^{A43}X^{A53}X^{A63}X^{A73}X^{A83}X^{A93}X^{A103} (A-2)
wherein
X^{A33} represents T or S,
X^{A43} represents F, L, or I,
X^{A53} represents D, S, N, or T,
X^{A63} represents D, N, S, or T,
X^{A73} represents N or S,
X^{A83} represents G or Y,
X^{A93} represents Y, F, or N,
X^{A103} represents T, G, W, or Y;
   a heavy-chain CDR2 containing the amino acid sequence represented by the following formula (B-1) or (B-2):

IX^{B21}X^{B31}X^{B41}X^{B51}X^{B61}X^{B71}X^{B81} (B-1)
wherein
X^{B21} represents S, N, D, W, or H,
X^{B31} represents Y, G, P, or S,
X^{B41} represents S, D, or G,
X^{B51} represents G, S, T, or A,
X^{B61} represents G, D, T, N, or R,
X^{B71} represents R, K, T, S, N, G, Y, or D,
X^{B81} represents T, I, or M, and

IX^{B23}X^{B33}X^{B43}X^{B53}X^{B63}X^{B73} (B-2)
wherein
X^{B23} represents S, N, D, W, or H,
X^{B33} represents Y, G, P, or S,
X^{B43} represents S, D, or G,
X^{B53} represents G, S, T, or A,
X^{B63} represents R, K, T, S, N, G, Y, or D,
X^{B73} represents T, I, or M;
   a heavy-chain CDR3 containing the amino acid sequence represented by any one of the following formulas (C-1) to (C-8)

X^{C11}X^{C21}X^{C31}X^{C41}X^{C51}X^{C61}X^{C71}X^{C81}X^{C91}X^{C101}X^{C111}X^{C121}X^{C131}X^{C141}X^{C151} (C-1)
wherein
X^{C11} represents A or S,
X^{C21} represents R, T, or K,
X^{C31} represents D, N, S, or Y,
X^{C41} represents G, S, or Y,
X^{C51} represents G, I, or D,
X^{C61} represents S or I,
X^{C71} represents Y, V, or A,
X^{C8} represents Y, H, S, T, K, or E,
X^{C91} represents S, I, or D,
X^{C101} represents P or Y,
X^{C111} represents I, Y, G, P, T, A, or V,
X^{C121} represents N, S, Y, P, G, or F,
X^{C131} represents F, C, M, or L,
X^{C141} represents Q or D,
X^{C151} represents F, Y, V, or S,

X^{C13}X^{C23}X^{C33}X^{C43}X^{C53}X^{C63}X^{C73}X^{C83} (C-2)
wherein
X^{C13} represents A or S,
X^{C23} represents R, T, or K,
X^{C33} represents D, N, S, or Y,
X^{C43} represents I, Y, G, P, T, A, or V,
X^{C53} represents N, S, Y, P, G, or F,
X^{C63} represents F, C, M, or L,
X^{C73} represents Q or D,
X^{C83} represents F, Y, V, or S,

X^{C15}X^{C25}X^{C35}X^{C45}X^{C55}X^{C65}X^{C75}X^{C85}X^{C95}TIFTFX^{C155}X^{C165}X^{C175}X^{C185}X^{C195} (C-3)
wherein
X^{C15} represents A or S,
X^{C25} represents R, T, or K,
X^{C35} represents G, S, or Y,
X^{C45} represents G, I, or D,
X^{C55} represents S or I,
X^{C65} represents Y, V, or A,
X^{C75} represents Y, H, S, T, K, or E,
X^{C85} represents S, I, or D,
X^{C95} represents P or Y,
X^{C155} represents I, Y, G, P, T, A, or V,
X^{C165} represents N, S, Y, P, G, or F,
X^{C175} represents F, C, M, or L,
X^{C185} represents Q or D,
X^{C195} represents F, Y, V, or S,

ARLWSGYALDI (C-4),

X^{C16}X^{C26}X^{C36}X^{C46}X^{C56}X^{C66}X^{C76}X^{C86}X^{C96}X^{C106}X^{C116}X^{C126}X^{C136} (C-5)
wherein
X^{C16} represents A or S,
X^{C26} represents R, T, or K,
X^{C36} represents D, N, S, or Y,
X^{C46} represents G, S, or Y,
X^{C56} represents G, I, or D,
X^{C66} represents Y, H, S, T, K, or E,
X^{C76} represents S, I, or D,
X^{C86} represents P or Y,
X^{C96} represents I, Y, G, P, T, A, or V,
X^{C106} represents N, S, Y, P, G, or F,
X^{C116} represents F, C, M, or L,
X^{C126} represents Q or D,
X^{C136} represents F, Y, V, or S,

V^{C27}PLLVX^{C77}HGX^{C107}X^{C117}X^{C127}X^{C137}X^{C147} (C-6)
wherein
X^{C27} represents S or K,
X^{C77} represents V, Y, or L,
X^{C107} represents I, Y, G, P, T, A, or V,
X^{C117} represents N, S, Y, P, G, or F,
X^{C127} represents F, C, M, or L,
X^{C137} represents Q or D,
X^{C147} represents F, Y, V, or S,

VSPSX^{C58}X^{C68}X^{C78}X^{C88}X^{C98}X^{C108} (C-7)
wherein
X^{C58} represents G or A,
X^{C68} represents I, Y, G, P, T, A, or V,
X^{C78} represents N, S, Y, P, G, or F,
X^{C88} represents F, C, M, or L,
X^{C98} represents Q or D,
X^{C108} represents F, Y, V, or S, and

SPSX^{C49}X^{C59}X^{C69}X^{C79}X^{C89}X^{C99} (C-8)
wherein
X^{C49} represents G or A,
X^{C59} represents I, Y, G, P, A, or V,
X^{C69} represents N, S, Y, P, G, or F,
X^{C79} represents F, C, M, or L,
X^{C89} represents Q or D,
X^{C99} represents F, Y, V, or S;
   a light-chain CDR1 containing the amino acid sequence represented by any one of the following formulas (D-1) to (D-3)

SX^{D21}X^{D31}X^{D41}X^{D51}X^{D61}X^{D71} (D-1)
wherein
X^{D21} represents E, D, Q, or G,
X^{D31} represents H, Y, G, or S,
X^{D41} represents S, I, K, or L,
X^{D51} represents H, N, or F,
X^{D61} represents Y or K,
X^{D71} represents Y, S, W, or N,

SX^{D23}X^{D33}X^{D43}X^{D53}X^{D63}X^{D73}GKNKDL (D-2)
wherein
X^{D23} represents E, D, Q, or G,
X^{D33} represents H, Y, G, or S,
X^{D43} represents R, S, I, K, or L,
X^{D53} represents H, N, or F,
X^{D63} represents Y or K,
X^{D73} represents Y, S, W, or N, and

NIGGKT (D-3);
a light-chain CDR2 containing the amino acid sequence represented by any one of the following formulas (E-1) to (E-3)

X^{E11}X^{E21}SX^{E41}GSX^{E71} (E-1)
wherein
X^{E11} represents L, V, or I,
X^{E21} represents K, N, or E,
X^{E41} represents D, Y, or K,
X^{E71} represents H or L,

VGTSDIVG (E-2), and

X^{E12}X^{E22}X^{E32} (E-3)
wherein
X^{E12} represents K, S, or W,
X^{E22} represents A or H,
X^{E32} represents N or S; and
   a light-chain CDR3 containing the amino acid sequence represented by any one of the following formulas (F-1) to (F-7):

GX^{F21}X^{F31}X^{F41}X^{F51}X^{F61}X^{F71}X^{F81}X^{F91}X^{F101} (F-1)
wherein
X^{F21} represents V or A,
X^{F31} represents S, D, N, G, or K,
X^{F41} represents Y, F, V, L, or H,
X^{F51} represents S, K, or N,
X^{F61} represents G or N,
X^{F71} represents A, E, or K,
X^{F81} represents S, Y, or V,
X^{F91} represents N, G, or S,
X^{F101} represents V, L, or Y,

GX^{F23}X^{F33}X^{F43}X^{F53}X^{F63}X^{F73}X^{F83}X^{F93}YX^{F113} (F-2)
wherein
X^{F23} represents V or A,
X^{F33} represents S, D, N, or G,
X^{F43} represents Y, F, V, L, or H,
X^{F53} represents S, K, or N,
X^{F63} represents G or N,
X^{F73} represents A, E, or K,
X^{F83} represents S, or Y,
X^{F93} represents N, G, or S,
X^{F113} represents V, L, or Y,

GX^{F24}X^{F34}X^{F44}X^{F54}IX^{F74}X^{F84}X^{F94}YX^{F114}YX^{F134} (F-3)
wherein
X^{F24} represents V or A,
X^{F34} represents S, D, N, or G,
X^{F44} represents Y, F, V, L, or H,
X^{F54} represents S, K, or N,
X^{F74} represents G or N,
X^{F84} represents A, E, or K,
X^{F94} represents S, or Y,
X^{F114} represents N, G, or S,
X^{F134} represents V, L, or Y,

GX^{F25}X^{F35}X^{F45}X^{F55}X^{F65}X^{F75}X^{F85}X^{F95} (F-4)
wherein
X^{F25} represents V or A,
X^{F35} represents S, D, N, G, or K,
X^{F45} represents Y, F, V, L, or H,
X^{F55} represents S, K, or N,
X^{F65} represents G or N,
X^{F75} represents A, E, or K,
X^{F85} represents S, Y, or V,
X^{F105} represents V, L, or Y,

QYDSTPPLT (F-5),

QVWDSYTYV (F-6), and

QFINGPLT (F-7).

### Item 3.

The antibody or fragment thereof according to Item 2, comprising
a heavy-chain CDR1 containing the amino acid sequence represented by formula (A-1),
a heavy-chain CDR2 containing the amino acid sequence represented by formula (B-1) or (B-2),
a heavy-chain CDR3 containing the amino acid sequence represented by any one of formulas (C-1), (C-2), (C-4), and (C-6) to (C-8),
a light-chain CDR1 containing the amino acid sequence represented by formula (D-1),
a light-chain CDR2 containing the amino acid sequence represented by any one of formulas (E-1) to (E-3), and
a light-chain CDR3 containing the amino acid sequence represented by any one of formulas (F-1) to (F-5).

### Item 4.

An antibody or fragment thereof (antigen-binding fragment), comprising
a heavy-chain CDR1 containing the amino acid sequence represented by the following formula (A-1-1):

GFX^{A32}X^{A42}X^{A52}X^{A62}X^{A72}X^{A82} (A-1-1)

wherein
X^{A32} represents T or S,
X^{A42} represents F or L,
X^{A52} represents D, S, N, or T,
X^{A62} represents D, S, or N,
X^{A72} represents Y or F, and
X^{A82} represents G, W, or Y;
a heavy-chain CDR2 containing the amino acid sequence represented by the following formula (B-1-1) or (B-2-1):

IX^{B22}X^{B32}X^{B42}X^{B52}X^{B62}X^{B72}X^{B82} (B-1-1)

wherein
X^{B22} represents S, N, D, or H,
X^{B32} represents Y, G, or P,
X^{B42} represents S or D,
X^{B52} represents G or S,
X^{B62} represents G, D, or N,
X^{B72} represents R, K, T, S, G, or Y,
X^{B82} represents T or I, and

IWX^{B34}X^{B44}X^{B54}X^{B64}X^{B74} (B-2-1)

wherein
X^{B34} represents Y or S.
X^{B44} represents D or G,
X^{B54} represents G or A,
X^{B64} represents R or D, and
X^{B74} represents T or I,
a heavy-chain CDR3 containing the amino acid sequence represented by any one of the following formulas (C-1-1), (C-2-1), (C-6-1), (C-7-1), and (C-8-1):

X^{C12}RDGGSYX^{C82}SPIX^{c122}X^{C132}QX^{C152} (C-1-1)

wherein
X^{C12} represents A or S,
X^{C82} represents Y, H, or S,
X^{C122} represents N or S,
X^{C132} represents F or C,
X^{C152} represents F or Y, and

ATX^{C34}X^{C44}X^{C54}X^{C64}X^{C74}X^{C84} (C-2-1)

wherein
X^{C34} represents N or S,
X^{C44} represents Y or G,
X^{C54} represents N or P,
X^{C64} represents F or M,
X^{C74} represents Q or D,
X^{C84} represents Y or V,

VSPLLVVHGPFFQY (C-6-1),

VSPSGAGLDV (C-7-1), and

SPSAVGFDV (C-8-1);
a light-chain CDR1 containing the amino acid sequence represented by any one of the following formulas (D-1-1) to (D-1-7)

SEYKHYN (D-1-1),

SEHRSYY (D-1-2),

SDYSHYS (D-1-3),

SQGINKW (D-1-4),

SGHSSYY (D-1-5),

SEHSSYY (D-1-6), and

SDHSSYY (D-1-7);
a light-chain CDR2 containing the amino acid sequence represented by any one of the following formulas (E-1-1), (E-2), and (E-3-1)

X^{E12}X^{E22}SDGSX^{E72} (E-1-1)

wherein
X^{E12} represents L, V, or I,
X^{E22} represents K, or E,
X^{E72} represents H or L,

VGTSDIVG (E-2), and

KAN (E-3-1); and
a light-chain CDR3 containing the amino acid sequence represented by any one of the following formulas (F-1-1), (F-2-1), (F-3-1), (F-4-1), and (F-5):

GVSX^{F42}SGGHX^{F92}X^{F102} (F-1-1)

wherein
X^{F42} represents Y or F,
X^{F92} represents N or G,
X^{F102} represents I or V,

GADYSGASSYV (F-2-1),

GANHNIGESYGYV (F-3-1),

GVX^{F36}YX^{F56}X^{F66}X^{F76}X^{F86}X^{F96} (F-4-1)

wherein
X^{F36} represents S, G, or K,
X^{F56} represents S or N,
X^{F66} represents G or N,
X^{F76} represents G or K,
X^{F86} represents Y or V,
X^{F96} represents V or Y; and

QYDSTPPLT (F-5).

### Item 5.

The antibody or fragment thereof according to Item 2, comprising
a heavy-chain CDR1 containing the amino acid sequence of any one of SEQ ID NOs: 3 to 11 or an amino acid sequence formed of any of these amino acid sequences with one to three amino acid mutations,
a heavy-chain CDR2 containing the amino acid sequence of any one of SEQ ID NOs: 12 to 25 or an amino acid sequence formed of any of these amino acid sequences with one to three amino acid mutations,
a heavy-chain CDR3 containing the amino acid sequence of any one of SEQ ID NOs: 26 to 41 or an amino acid sequence formed of any of these amino acid sequences with one to three amino acid mutations,
a light-chain CDR1 containing the amino acid sequence of any one of SEQ ID NOs: 42 to 50 or an amino acid sequence formed of any of these amino acid sequences with one to three amino acid mutations,
a light-chain CDR2 containing the amino acid sequence of any one of SEQ ID NOs: 51 to 58 or an amino acid sequence formed of any of these amino acid sequences with one to three amino acid mutations, or the amino acid sequence represented by KAN, SHN, or WAS, and
a light-chain CDR3 containing the amino acid sequence of any one of SEQ ID NOs: 59 to 73 or an amino acid sequence formed of any of these amino acid sequences with one to three amino acid mutations.

### Item 5a.

The antibody or fragment thereof according to Item 2, comprising
a heavy-chain CDR1 containing the amino acid sequence of any one of SEQ ID NOs: 3 to 9 or an amino acid sequence formed of any of these amino acid sequences with one to three amino acid mutations,
a heavy-chain CDR2 containing the amino acid sequence of any one of SEQ ID NOs: 12 to 19 and 25 or an amino acid sequence formed of any of these amino acid sequences with one to three amino acid mutations,
a heavy-chain CDR3 containing the amino acid sequence of any one of SEQ ID NOs: 26 to 29, 32, 33, 37, 40, and 41 or an amino acid sequence formed of any of these amino acid sequences with one to three amino acid mutations,
a light-chain CDR1 containing the amino acid sequence of any one of SEQ ID NOs: 42 to 48 or an amino acid sequence formed of any of these amino acid sequences with one to three amino acid mutations,
a light-chain CDR2 containing the amino acid sequence of any one of SEQ ID NOs: 51 to 54 and 57 or an amino acid sequence formed of any of these amino acid sequences with one to three amino acid mutations, or the amino acid sequence represented by KAN, and
a light-chain CDR3 containing the amino acid sequence of any one of SEQ ID NOs: 59 to 61, 64 to 68, and 71 or an amino acid sequence formed of any of these amino acid sequences with one to three amino acid mutations.

### Item 6.

The antibody or fragment thereof according to Item 5, wherein the mutations are conservative substitutions.

### Item 6a.

The antibody or fragment thereof according to Item 5a, wherein the mutations are conservative substitutions.

### Item 7.

The antibody or fragment thereof according to any one of Items 2 to 6, which is specifically bindable to the amino acid sequence of SEQ ID NO: 1.

### Item 7a.

The antibody or fragment thereof according to Item 5a or 6a, which is specifically bindable to the amino acid sequence of SEQ ID NO: 1.

### Item 8.

An antibody or fragment thereof (antigen-binding fragment), comprising
a heavy-chain CDR1 containing the amino acid sequence represented by the following formula (G-1) or (G-2):

GX^{G21}X^{G31}X^{G41}X^{G51}X^{G61}X^{G71}X^{G81} (G-1)

wherein
X^{G21} represents F or L,
X^{G31} represents I, T, S, or F,
X^{G41} represents F or L,
X^{G51} represents S, N, T, R, or D,
X^{G61} represents N, T, or D,
X^{G71} represents Y, S, or H,
X^{G81} represents F, T, Y, W, or G,

KLSERY (G-2);
a heavy-chain CDR2 containing the amino acid sequence represented by any one of the following formulas (H-1) to (H-3)

IX^{H21}X^{H31}X^{H41}X^{H51}X^{H61}X^{H71}X^{H81} (H-1)

wherein
X^{H21} represents S, G, K, or N,
X^{H31} represents G, N, Y, T, or P,
X^{H41} represents D, S, or A,
X^{H51} represents G, or D,
X^{H61} represents S, A, G, or R,
X^{H71} represents Y, K, N, T, I, or S,
X^{H81} represents I, T, P, or V,

MWSDGDT (H-2), and

NDS (H-3);
a heavy-chain CDR3 containing the amino acid sequence represented by any one of the following formulas (I-1) to (I-11):

X^{I11}X^{I21}X^{I31}X^{I41}X^{I31}X^{I61}X^{I71} (I-1)

wherein
X^{I11} represents S or T,
X^{I21} represents I or T,
X^{I31} represents N, L, D, or T,
X^{I41} represents W or G,
X^{I51} represents G, V, F, or N,
X^{I61} represents D, G, F, or N,
X^{I71} represents V or L,

ARTSRIVADLVTMGYALDF (I-2),

ARDVVKTGTTGLPFDL (I-3),

AREGVITVGTWGDV (I-4),

ATSEY (I-5),

TTATDV (I-6),

TPATDV (I-7),

SPMTIHGLDT (I-8),

GSGWV (I-9),

GSGWY (I-10), and

LSSESDDARV (I-11);
a light-chain CDR1 containing the amino acid sequence represented by any one of the following formulas (J-1) to (J-3):

XJIILX^{J31}X^{J41}X^{J51}Y (J-1)
wherein
X^{J11} represents K or A,
X^{J31} represents S or
X^{J41} represents N, E, K, T, or D,
X^{J51} represents Q, R, K, or E,

SX^{J23}SLX^{J53}X^{J63}X^{J73}DGX^{J103}TX^{J123} (J-2),

wherein
X^{J23} represents E or
X^{J33} represents L or V,
X^{J63} represents H, D, or Y,
X^{J73} represents S or G,
X^{J103} represents N, K, or Y,
X^{J123} represents Y or F, and

NIGGKA (J-3),
a light-chain CDR2 containing the amino acid sequence represented by the following formula (K-1):

X^{K11}X^{K21}X^{K31} (K-1)

wherein
X^{K11} represents K, N, L, or E,
X^{K21} represents D, G, V, or I,
X^{K31} represents S, N, or D, and
a light-chain CDR3 containing the amino acid sequence represented by any one of the following formulas (L-1) to (L-6)

LSX^{L31}X^{L41}X^{L51}X^{L61}X^{L71}X^{L81}X^{L91}X^{L101}X^{L111} (L-1)

wherein
X^{L31} represents R, S, A, or G,
X^{L41} represents Q, E, or D,
X^{L51} represents S or G,
X^{L61} represents D, S, or N,
X^{L71} represents D, G, or T,
X^{L81} represents A, T, or Y,
X^{L91} represents A, T, or Y,
X^{L101} represents S, R, or W,
X^{L111} represents V, L, or Y,

LSX^{L33}X^{L43}X^{L53}X^{L63}X^{L73}X^{L83}X^{L93}X^{L103} (L-2)

wherein
X^{L33} represents R, S, A, or G,
X^{L43} represents Q, E, or D,
X^{L53} represents S or G,
X^{L63} represents D, S, or N,
X^{L73} represents D, G, or T,
X^{L83} represents A, T, or Y,
X^{L93} represents S, R, or W,
X^{L103} represents V, L, or Y,

X^{L14}QX^{L34}X^{L44}X^{L54}X^{L64}PX^{L84}X^{L94} (L-3)

wherein
X^{L14} represents L or V,
X^{L34} represents A or T,
X^{L44} represents T or S,
X^{L54} represents H or N,
X^{L64} represents D, N, or V,
X^{L84} represents Y, V, I, or L,
X^{L94} represents T or S,

QX^{L27}X^{L37}X^{L47}X^{L57}PX^{L77}X^{L87} (L-4)

wherein
X^{L27} represents A or T,
X^{L37} represents T or S,
X^{L47} represents H or N,
X^{L57} represents D, N, or V,
X^{L77} represents Y, V, I, or L,
X^{L87} represents T or S,

QVYDSSSFV (L-5), and

WQGTDFPR (L-6).

### Item 9.

The antibody or fragment thereof according to Item 8, comprising
a heavy-chain CDR1 containing the amino acid sequence represented by formula (G-1),
a heavy-chain CDR2 containing the amino acid sequence represented by formula (H-1) or (H-2),
a heavy-chain CDR3 containing the amino acid sequence represented by any one of formulas (I-1) to (I-3) and (I-5) to (1-9),
a light-chain CDR1 containing the amino acid sequence represented by any one of formulas (J-1) to (J-3),
a light-chain CDR2 containing the amino acid sequence represented by formula (K-1), and
a light-chain CDR3 containing the amino acid sequence represented by any one of formulas (L-1) to (L-3), (L-5), and (L-6).

### Item 10.

An antibody or fragment thereof (antigen-binding fragment), comprising
a heavy-chain CDR1 containing the amino acid sequence represented by the following formula (G-1-1):

GX^{G22}X^{G32}X^{G42}X^{G52}X^{G62}X^{G72}X^{G82} (G-1-1)

wherein
X^{G22} represents F or L,
X^{G32} represents I, T, or S,
X^{G42} represents F or L,
X^{G52} represents S, N, T, or R,
X^{G62} represents N or T,
X^{G72} represents Y, S, or H,
X^{G82} represents F, T, Y, or W,
a heavy-chain CDR2 containing the amino acid sequence represented by the following formula (H-1-1) or (H-2):

IX^{H22}X^{H32}X^{H42}X^{H52}X^{H62}X^{H72}X^{H82} (H-1-1)

wherein
X^{H22} represents S, G, K, or N,
X^{H32} represents G, N, S, T, or P,
X^{H42} represents D, S, or A,
X^{H52} represents G, or D,
X^{H62} represents S, G, or R,
X^{H72} represents Y, K, N, T, I, or S,
X^{H82} represents I, T, P, or V, and

MWSDGDT (H-2),
a heavy-chain CDR3 containing the amino acid sequence represented by any one of the following formulas (I-1-1), (I-2), (I-3), and (I-5) to (I-9):

X^{I12}X^{I22}X^{I32}X^{I42}X^{I52}X^{I62}X^{I72} (I-1-1)

wherein
X^{I12} represents S or T,
X^{I22} represents I or T,
X^{I32} represents N, L, or T,
X^{I42} represents W or G,
X^{I52} represents G, V, F, or N,
X^{I62} represents D, F, or G,
X^{I72} represents V or L,

ARTSRIVADLVTMGYALDF (I-2),

ARDVVKTGTTGLPFDL (I-3),

ATSEY (I-5),

TTATDV (I-6),

TPATDV (I-7),

SPMTIHGLDT (I-8), and

GSGWV (I-9),
a light-chain CDR1 containing the amino acid sequence represented by any one of the following formulas (J-1-1), (J-2-1), and (J-3):

KLSX^{J42}X^{J52}Y (J-1-1)

wherein
X^{J42} represents N, E, or K,
X^{J52} represents Q, R, or E,

SX^{J24}SLX^{J54}X^{J64}SDGX^{J104}TX^{J124} (J-2-1)

wherein
X^{J24} represents E or Q,
X^{J34} represents L or V,
X^{J64} represents H, D, or Y,
X^{J104} represents N, K, or Y,
X^{J124} represents Y or F, and

NIGGKA (J-3),
a light-chain CDR2 containing the amino acid sequence represented by the following formulas (K-1-1) to (K-1-7):

NDD (K-1-1),

KDS (K-1-2),

NGN (K-1-3),

NDS (K-1-4),

KVS (K-1-5),

LIS (K-1-6), and

EVS (K-1-7), and
a light-chain CDR3 containing the amino acid sequence represented by any one of the following formulas (L-1-1), (L-2-1), (L-3-1), (L-5), and (L-6)

LSX^{L32}X^{L42}SX^{L62}DA X^{L102}V (L-1-1)

wherein
X^{L32} represents R or S,
X^{L42} represents Q or E,
X^{L62} represents D or N,
X^{L102} represents S, R, or W,

LSGESDDAWV (L-2-1),

X^{L15}QX^{L35}THDPYT (L-3-1)

wherein
X^{L15} represents L or V,
X^{L35} represents A or T,

QVYDSSSFV (L-5), and

WQGTDFPR (L-6).

### Item 11.

The antibody or fragment thereof according to Item 8, comprising
a heavy-chain CDR1 containing the amino acid sequence of any one of SEQ ID NOs: 74 to 84 or an amino acid sequence formed of any of these amino acid sequences with one to three amino acid mutations,
a heavy-chain CDR2 containing the amino acid sequence of any one of SEQ ID NOs: 85 to 101 or an amino acid sequence formed of any of these amino acid sequences with one to three amino acid mutations, or the amino acid sequence represented by NDS,
a heavy-chain CDR3 containing the amino acid sequence of any one of SEQ ID NOs: 102 to 118 or an amino acid sequence formed of any of these amino acid sequences with one to three amino acid mutations,
a light-chain CDR1 containing the amino acid sequence of any one of SEQ ID NOs: 119 to 133 or an amino acid sequence formed of any of these amino acid sequences with one to three amino acid mutations,
a light-chain CDR2 containing the amino acid sequence represented by NDD, KDS, NGN, NDS, KVS, LIS, or EVS, and
a light-chain CDR3 containing the amino acid sequence of any one of SEQ ID NOs: 134 to 149 or an amino acid sequence formed of any of these amino acid sequences with one to three amino acid mutations.

### Item 11a.

The antibody or fragment thereof according to Item 8, comprising
a heavy-chain CDR1 containing the amino acid sequence of any one of SEQ ID NOs: 74 to 81 or an amino acid sequence formed of any of these amino acid sequences with one to three amino acid mutations,
a heavy-chain CDR2 containing the amino acid sequence of any one of SEQ ID NOs: 85 to 90, 92 to 95, and 101 or an amino acid sequence formed of any of these amino acid sequences with one to three amino acid mutations,
a heavy-chain CDR3 containing the amino acid sequence of any one of SEQ ID NOs: 102 to 106, 109, 110, and 112 to 116 or an amino acid sequence formed of any of these amino acid sequences with one to three amino acid mutations,
a light-chain CDR1 containing the amino acid sequence of any one of SEQ ID NOs: 119 to 121, 125 to 129, and 133 or an amino acid sequence formed of any of these amino acid sequences with one to three amino acid mutations,
a light-chain CDR2 containing the amino acid sequence represented by NDD, KDS, NGN, NDS, KVS, LIS, or EVS, and
a light-chain CDR3 containing the amino acid sequence of any one of SEQ ID NOs: 134 to 136, 140 to 142, 148, and 149 or an amino acid sequence formed of any of these amino acid sequences with one to three amino acid mutations.

### Item 12.

The antibody or fragment thereof according to Item 11, wherein the mutations are conservative substitutions.

### Item 12a.

The antibody or fragment thereof according to Item 11a, wherein the mutations are conservative substitutions.

### Item 13.

The antibody or fragment thereof according to any one of Items 8 to 12, which is specifically bindable to the amino acid sequence of SEQ ID NO: 2.

### Item 13a.

The antibody or fragment thereof according to Item 11a or 12a, which is specifically bindable to the amino acid sequence of SEQ ID NO: 2.

### Item 14.

An antibody or fragment thereof (antigen-binding fragment) against a nucleocapsid protein of severe acute respiratory syndrome coronavirus-2 (SARS-CoV-2), whose epitope is present in a region formed of the amino acid sequence of any of SEQ ID NOs: 150 to 152 of the nucleocapsid protein.

### Item 15.

The antibody or fragment thereof according to any one of Items 1 to 14, wherein the antibody is a monoclonal antibody.

### Item 15a.

The antibody or fragment thereof according to any one of Items 5a, 6a, 11a, and 12a, wherein the antibody is a monoclonal antibody.

### Item 16.

A polynucleotide comprising a coding sequence of the antibody or fragment thereof of any one of Items 1 to 15.

### Item 16a.

A polynucleotide comprising a coding sequence of the antibody or fragment thereof of any one of Items 5a, 6a, 11a, 12a, and 15a.

### Item 17.

A cell comprising the polynucleotide of Item 16.

### Item 17a.

A cell comprising the polynucleotide of Item 16a.

### Item 18.

A reagent or drug comprising the antibody or fragment thereof of any one of Items 1 to 15, the polynucleotide of Item 16, or the cell of Item 17.

### Item 18a.

A reagent or drug comprising the antibody or fragment thereof of any one of Items 5a, 6a, 11a, 12a, and 15a, the polynucleotide of Item 16a, or the cell of Item 17a.

### Item 19.

A kit for detecting the nucleocapsid protein of severe acute respiratory syndrome coronavirus-2 (SARS-CoV-2), comprising the antibody or fragment thereof of any one of Items 1 to 15.

### Item 19a.

A kit for detecting the nucleocapsid protein of severe acute respiratory syndrome coronavirus-2 (SARS-CoV-2), comprising the antibody or fragment thereof of any one of Items 5a, 6a, 11a, 12a, and 15a.

### Item 20.

A kit for detecting the nucleocapsid protein of severe acute respiratory syndrome coronavirus-2 (SARS-CoV-2), comprising
two or more antibodies or fragments thereof specifically bindable to the amino acid sequence of SEQ ID NO: 1, the two or more antibodies or fragments thereof each recognizing a different epitope in the amino acid sequence of SEQ ID NO: 1, and/or
two or more antibodies or fragments thereof specifically bindable to the amino acid sequence of SEQ ID NO: 2, the two or more antibodies or fragments thereof each recognizing a different epitope in the amino acid sequence of SEQ ID NO: 2.

### Item 21.

The kit according to Item 20,
wherein
   the two or more antibodies or fragments thereof specifically bindable to the amino acid sequence of SEQ ID NO: 1 are each the antibody or fragment thereof of any one of Items 2 to 7 or an antibody or fragment thereof containing an amino acid sequence with at least 90% identity to the amino acid sequence of the antibody or fragment thereof of any one of Items 2 to 7, and
   the two or more antibodies or fragments thereof specifically bindable to the amino acid sequence of SEQ ID NO: 2 are each the antibody or fragment thereof of any one of Items 8 to 14 or an antibody or fragment thereof containing an amino acid sequence with at least 90% identity to the amino acid sequence of the antibody or fragment thereof of any one of Items 8 to 14.

### Item 21a.

The kit according to Item 20,
wherein
   the two or more antibodies or fragments thereof specifically bindable to the amino acid sequence of SEQ ID NO: 1 are each the antibody or fragment thereof of Item 5a or 6a, and
   the two or more antibodies or fragments thereof specifically bindable to the amino acid sequence of SEQ ID NO: 2 are each the antibody or fragment thereof of Item 11a or 12a.

### Item 22.

The kit according to any one of Items 19 to 21, for detecting the nucleocapsid protein of severe acute respiratory syndrome coronavirus-2 (SARS-CoV-2) by immunochromatography, enzyme immunoassay, chemiluminescent immunoassay, chemiluminescent enzyme immunoassay, radioimmunoassay, electrochemiluminescent immunoassay, immunonephelometry, or latex agglutination.

### Item 22a.

The kit according to Item 19a or 21a, for detecting the nucleocapsid protein of severe acute respiratory syndrome coronavirus-2 (SARS-CoV-2) by immunochromatography, enzyme immunoassay, chemiluminescent immunoassay, chemiluminescent enzyme immunoassay, radioimmunoassay, electrochemiluminescent immunoassay, immunonephelometry, or latex agglutination.

### Item 23.

The kit according to any one of Items 19 to 22, wherein a portion of two or more antibodies or fragments thereof specifically bindable to the amino acid sequence of SEQ ID NO: 1 or 2 is immobilized, and the remaining portion is labeled.

### Item 23a.

The kit according to any one of Items 19a, 21a, and 22a, wherein a portion of two or more antibodies or fragments thereof specifically bindable to the amino acid sequence of SEQ ID NO: 1 or 2 are immobilized, and the remaining portion is labeled.

### Item 24.

A method for detecting the nucleocapsid protein of severe acute respiratory syndrome coronavirus-2 (SARS-CoV-2), comprising the following steps (1) to (3):
(1) immobilizing a primary antibody or fragment thereof specifically bindable to the amino acid sequence of SEQ ID NO: 1 or 2 onto a solid phase,
(2) applying a secondary antibody or fragment thereof that is specifically bindable to a nucleocapsid protein of SARS-CoV-2, and that is specifically bindable to the amino acid sequence of SEQ ID NO: 1 or 2 to the solid phase, wherein the secondary antibody or fragment thereof recognizes a different epitope in an amino acid sequence to which the primary antibody or fragment thereof is specifically bindable, and the secondary antibody or fragment thereof is labeled with a labeling substance, and
(3) detecting a label derived from the labeling substance of the secondary antibody or fragment thereof bound to the solid phase via the nucleocapsid protein.

### Item 25.

The method according to Item 24, wherein the primary antibody or fragment thereof and the secondary antibody or fragment thereof are selected from the group consisting of the antibody or fragment thereof of any one of Items 2 to 15, and an antibody or fragment thereof containing an amino acid sequence with at least 90% identity to the amino acid sequence of the antibody or fragment thereof of any one of Items 2 to 15.

### Item 25a.

The method according to Item 24, wherein the primary antibody or fragment thereof and the secondary antibody or fragment thereof are selected from the group consisting of the antibody or fragment thereof of any one of Items 5a, 6a, 11a, 12a, and 15a, and an antibody or fragment thereof containing an amino acid sequence with at least 90% identity to the amino acid sequence of the antibody or fragment thereof of any one of Items 5a, 6a, 11a, 12a, and 15a.

### Advantageous Effects of Invention

The present invention provides an antibody or fragment thereof specifically bindable to the NTD or CTD of the N protein of SARS-CoV-2. The present invention also provides a kit and a method for detecting the N protein of SARS-CoV-2 with high sensitivity.

### DESCRIPTION OF EMBODIMENTS

### 1. Definition

In the present specification, an amino acid may be a natural or non-natural amino acid. Examples of non-natural amino acids include, but are not limited to, citrulline, ornithine, ε-acetyl-lysine, β-alanine, aminobenzoic acid, 6-aminocaproic acid, aminobutyric acid, hydroxyproline, mercaptopropionic acid, 3-nitrotyrosine, norleucine, and pyroglutamic acid. An amino acid may be, for example, L-amino acid, D-amino acid, or DL-amino acid.

In the present specification, the “identity” of amino acid sequences refers to the degree to which two or more contrastable amino acid sequences match each other in optimal alignment. The identity of amino acid sequences can be calculated by using analysis tools available commercially or via telecommunication lines (Internet), such as commercial software GENETYX (Genetyx Corporation) or using default parameters in the homology algorithm BLAST (basic local alignment search tool) of the National Center for Biotechnology Information (NCBI) (http://www.ncbi.nlm.nih.gov/BLAST/).

The amino acid sequences disclosed in the present specification may have one or more (e.g., one, two, or three) amino acids deleted, substituted, or modified, and may have one or more (e.g., one, two, or three) amino acids inserted or added as long as its bindability to the N protein of SARS-CoV-2 is not interfered with.

The substitution of an amino acid is preferably a substitution of an amino acid to another amino acid similar in structure and/or properties (conservative substitution). Conservative substitution includes, for example, substitutions within the groups shown in Table 1.

TABLE 1

| | | | Group |
|---|---|---|---|
| Classification | Non- | H or Unbranched Hydrocarbon | Glycine (G), Alanine (A) |
| by Side Chain | aromatic | β-Branched Hydrocarbon | Valine (V), Leucine (L), Isoleucine (I) |
| Structure | | Oxygen-containing | Serine (S), Threonine (T) |
| | | Sulfur-containing | Cysteine (C), Methionine (M) |
| | | Nitrogen-containing | Lysine (K), Arginine (R), Asparagine (N), Glutamine |
| | | | (Q), Proline (P) |
| | Aromatic | | Phenylalanine (F), Tryptophan (W), Histidine (H), |
| | | | Tyrosine (Y) |
| Classification | Hydrophobic | | Alanine (A), Valine (V), Leucine (L), Isoleucine (I), |
| Charge/Polarity | Non-polar | | Proline (P), Phenylalanine (F), Methionine (M), |
| in Side Chain | | | Tryptophan (W) |

| | |
|---|---|
| Uncharged | Glycine (G), Asparagine (N), Glutamine (Q), Serine (S), |
| Polar | Threonine (T), Tyrosine (Y), Cysteine (C) |
| Acidic | Aspartic Acid (D), Glutamic Acid (E) |
| Basic | Lysine (K), Arginine (R), Histidine (H) |

Modification of amino acids includes, for example, modification of functional groups such as amino, carboxyl, hydroxyl, and sulfhydryl (SH) groups. Examples of modification of the functional groups include glycosylation; methylation; esterification; amidation; PEGylation; phosphorylation; hydroxylation; binding to protective groups such as a t-butoxycarbonyl (Boc) group or a 9-fluorenylmethyloxycarbonyl (Fmoc) group; biotinylation; binding to a fluorescent dye such as fluorescein isothiocyanate (FITC); and binding to an enzyme such as peroxidase (HRP) or alkaline phosphatase (ALP).

In the present specification, antibodies may be either a monoclonal antibody or a polyclonal antibody, and are preferably a monoclonal antibody. Antibodies can be of any isotype, such as IgG, IgA, IgD, IgE, or IgM. Antibodies may be either a human antibody or a non-human antibody. Non-human antibodies include, but are not limited to, mouse antibodies, rat antibodies, and guinea pig antibodies. Antibodies may be a chimeric antibody, such as a mouse-human chimeric antibody. Antibodies can be a partially or fully humanized antibody.

In the present specification, antibody fragments are not particularly limited as long as they include heavy-chain CDR1 to CDR3 and light-chain CDR1 to CDR3. Examples of antibody fragments include Fv, Fab, Fab′, (Fab′)₂, scFv, scFv-Fc, diabody, triabody, tetrabody, and minibody. Antibody fragments are preferably a fragment with an antigen-binding capacity (antigen-binding fragment).

In the present specification, the heavy-chain CDR1 to CDR3 and the light-chain CDR1 to CDR3 are defined according to IMGT.

In the present specification, nucleotides such as DNA and RNA may have known chemical modifications as illustrated below. To prevent degradation by hydrolytic enzymes such as nucleases, the phosphate residue (phosphate) of each nucleotide may be replaced with a chemically modified phosphate residue, such as phosphorothioate (PS), methyl phosphonate, or phosphorodithionate. The hydroxyl group at the 2-position of the ribose of each ribonucleotide may be replaced with —OR (R is, for example, —CH₃, —CH₂CH₂OCH₃, —CH₂CH₂NHC (NH) NH₂, —CH₂CONHCH₃, and —CH₂CH₂CN). Additionally, the base moiety (pyrimidine, purine) may be chemically modified, for example, by introducing a methyl group or cationic functional group at the 5-position of the pyrimidine base, or by replacing the carbonyl group at the 2-position with thiocarbonyl. Furthermore, the phosphate and hydroxyl moieties may be modified with, for example, biotin, an amino group, a lower alkylamine group, or an acetyl group. However, modifications are not limited to these.

In the present specification, the N protein of SARS-CoV-2 (antigen) has the amino acid sequence disclosed in GenBank accession number MN908947. The N protein of SARS-CoV has the amino acid sequence disclosed in GenBank accession number AY278741.

### 2. Antibody or Fragment Thereof Specifically Bindable to the NTD of the N Protein of SARS-CoV-2

The NTD of the N protein of SARS-CoV-2 (simply “NTD”) means the amino acid region at positions 1 to 172 from the N-terminus of the N protein and has the following amino acid sequence:

(SEQ ID NO: 1)
MSDNGPQNQRNAPRITFGGPSDSTGSNQNGERSGARSKQRRPQGLPNNTA
SWFTALTQHGKEDLKFPRGQGVPINTNSSPDDQIGYYRRATRRIRGGDGK
MKDLSPRWYFYYLGTGPEAGLPYGANKDGIIWVATEGALNTPKDHIGTRN
PANNAAIVLQLPQGTTLPKGFY

The antibody or fragment thereof specifically bindable to the NTD of SARS-CoV-2 preferably does not bind to the NTD of SARS-CoV or has low bindability to the NTD of SARS-CoV. In the amino acid sequence of SEQ ID NO: 1, for example, the 63rd D, 65th K, 79th S, 94th I, 103rd D, 120th G, 128th D, 131st I, 152nd A, and 157th I differ from their corresponding amino acid in the NTD of SARS-CoV. The antibody or fragment thereof specifically bindable to the NTD of SARS-CoV-2 preferably binds to a region (or an epitope) containing one or more amino acids selected from the group consisting of the 63rd D, 65th K, 79th S, 94th I, 103rd D, 120th G, 128th D, 131st I, 152nd A, and 157th I in SEQ ID NO: 1.

In an embodiment, the antibody or fragment thereof specifically bindable to the NTD of SARS-CoV-2 may bind to the amino acid region at positions 1 to 180 from the N-terminus of the N protein, preferably to the RNA-binding domain of the NTD, and preferably to the amino acid region at positions 44 to 180 or 44 to 172 from the N-terminus of the N protein.

The antibody or fragment thereof specifically bindable to the NTD of SARS-CoV-2 can be produced according to known methods in combination, such as by a method including the following steps (P1) to (P6):
(P1) obtaining a cell producing an antibody that specifically binds to the N protein of SARS-CoV-2 from antibody-producing cells of an animal immunized with the N protein of SARS-CoV-2 by using the N protein of SARS-CoV-2 labeled with a fluorescent label whose staining selectivity for the endoplasmic reticulum is higher than that for cell organelles other than the endoplasmic reticulum;
(P2) obtaining an antibody variable region gene fragment from the cell obtained in step (P1);
(P3) obtaining a gene expression unit from the antibody variable region gene fragment obtained in step (P2);
(P4) introducing the gene expression unit obtained in step (P3) into a cell to express antibodies that bind to the N protein of SARS-CoV-2;
(P5) recovering the antibodies expressed in step (P4); and
(P6) obtaining antibodies that specifically bind to the NTD from the antibodies recovered in step (P5).

Step (P1) can be performed according to the method (ERIAA method) described, for example, in U.S. Patent Application Publication No. 2013/0029325 (incorporated by reference in its entirety into this specification).

Step (P2) can be performed according to the method (MAGrand method) described, for example, in the following literature: Rapid production of antigen-specific monoclonal antibodies from a variety of animals, BMC Biology 2012, 10:80.

Step (P3) can be performed according to the method (TS-jPCT method) described, for example, in U.S. Patent Application Publication No. 2013/0023009 (incorporated by reference in its entirety into this specification).

Step (P4) can be performed using known gene transfer methods, such as calcium phosphate transfection, lipofection, DEAE dextran, electroporation, or microinjection. The cells into which the gene expression unit is introduced are not particularly limited to specific species as long as the cells can transiently express antibodies, and include mammalian cells such as CHO cells, HEK293 cells, Expi293 cells, 293F cells, 293T cells, and 293FT cells.

Step (P6) can be performed by selecting antibodies that specifically bind to the NTD according to a known screening method, such as enzyme-linked immunosorbent assay (ELISA), western blotting, or immunostaining.

The antibodies may optionally be purified according to a known method, such as salting out using ammonium sulfate, sodium sulfate, etc.; affinity chromatography using protein A etc.; ion-exchange chromatography using DEAE cellulose etc.; gel filtration; or a purification method using His-tag, FLAG-tag, etc.

The method including steps (P1) to (P6) can provide multiple types of antibodies or fragments thereof that specifically bind to the NTD of SARS-CoV-2. (The antibodies or fragments thereof differ from each other in terms of the amino acid sequence of at least one CDR of the heavy-chain CDR1 to CDR3 and the light-chain CDR1 to CDR3.)

In an embodiment, the heavy-chain CDR1 of the antibody or fragment thereof specifically bindable to the NTD of SARS-CoV-2 preferably contains the following amino acid sequence:
the amino acid sequence represented by either formula (A-1) or (A-2) shown in Table 2
an amino acid sequence having at least 90% (preferably at least 95%) identity to the amino acid sequence represented by either formula (A-1) or (A-2), or
an amino acid sequence formed of the amino acid sequence represented by either formula (A-1) or (A-2) with 1 to 3 (preferably 1 or 2, more preferably 1) amino acid mutations (preferably conservative substitution).

TABLE 2

| GFX^{A31}X^{A41}X^{A51}X^{A61}X^{A71}X^{A81} | GFX^{A32}X^{A42}X^{A52}X^{A62}X^{A72}X^{A82} | GFTFDDYG |
|---|---|---|
| (A-1) wherein | (A-1-1) | (A-1-2, SEQ ID NO: 3) |
| X^{A31} represents T or S, | X^{A32} represents T or S, | GFTFSDFW |
| X^{A41} represents F, L, or I, | X^{A42} represents F or L, | (A-1-3, SEQ ID NO: 4) |
| X^{A51} represents D, S, N, or T, | X^{A52} represents D, S, N, or T, | GFTFNNYW |
| X^{A61} represents D, N, S, I, or T, | X^{A62} represents D, S, or N, | (A-1-4, SEQ ID NO: 5) |
| X^{A71} represents Y, F, or N, | X^{A72} represents Y or F, | GFTFDDFG |
| X^{A81} represents G, W, Y, S, or T. | X^{A82} represents G, W, S, or Y. | (A-1-5, SEQ ID NO: 6) |
| | | GFTFSNYY |
| | | (A-1-6, SEQ ID NO: 7) |
| | | GFTFSDYW |
| | | (A-1-7, SEQ ID NO: 8) |
| | | GFSLTSYS |
| | | (A-1-8, SEQ ID NO: 9) |
| | GFTFSDYT (A-1-9, SEQ ID NO: 10) | |
| GFX^{A33}X^{A43}X^{A53}X^{A63}X^{A73}X^{A83}X^{A93}X^{A103} | GFSITTX^{A74} | GFSITTNGYT |
| (A-2) wherein | X^{A84}YX^{A104} | (A-2-2, SEQ ID NO: 11) |
| X^{A33} represents T or S, | (A-2-1) wherein | |
| X^{A43} represents F, L, or I, | X^{A74} represents N or S, | |
| X^{A53} represents D, S, N, or T, | X^{A84} represents G or Y, | |
| X^{A63} represents D, N, S, or T, | X^{A104} represents T or G. | |
| X^{A73} represents N or S, | | |
| X^{A83} represents G or Y, | | |
| X^{A93} represents Y, F, or N, | | |
| X^{A103} represents T, G, W, or Y | | |

In formula (A-1), X^{A41} is preferably F or L; X^{A51} is preferably D, S, N, or T; X^{A61} is preferably D, N, S, or I, and more preferably D, N, or S; X^{A71} is preferably Y or F; and X^{A81} is preferably G, W, S, or Y.

Formula (A-1) is preferably formula (A-1-1) or one selected from the group consisting of formulas (A-1-2) to (A-1-9), and more preferably one selected from the group consisting of formulas (A-1-2) to (A-1-8).

In formula (A-2), X^{A33} is preferably S; X^{A43} is preferably I; X^{A53} is preferably T; X^{A63} is preferably T; X^{A73} is preferably N or S; X^{A83} is preferably G or Y; X^{A93} is preferably Y or F, and more preferably Y; and X^{A103} is preferably T, G, or W, and more preferably is T or G.

Formula (A-2) is preferably formula (A-2-1), and more preferably formula (A-2-2).

The heavy-chain CDR1 of the antibody or fragment thereof specifically bindable to the NTD of SARS-CoV-2 preferably contains the amino acid sequence of formula (A-1); more preferably the amino acid sequence of formula (A-1-1) or one amino acid sequence selected from the group consisting of the amino acid sequences of formulas (A-1-2) to (A-1-9); and still more preferably one amino acid sequence selected from the group consisting of the amino acid sequences of formulas (A-1-2) to (A 1-8); or an amino acid sequence having at least 90% identity to any of these amino acid sequences. The identity is preferably at least 910, at least 92%, at least 93%, at least 94%, or at least 950.

In an embodiment, the heavy-chain CDR2 of the antibody or fragment thereof specifically bindable to the NTD of SARS-CoV-2 preferably contains the following amino acid sequence:
the amino acid sequence represented by either formula (B-1) or (B-2) shown in Table 3,
an amino acid sequence having at least 90% (preferably at least 95%) identity to the amino acid sequence represented by either formula (B-1) or (B-2), or
an amino acid sequence formed of the amino acid sequence represented by either formula (B-1) or (B-2) with 1 to 3 (preferably 1 or 2, more preferably 1) amino acid mutations (preferably conservative substitution).

TABLE 3

| IX^{B21}X^{B31}X^{B41}X^{B51}X^{B61}X^{B71}X^{B81} | IX^{B22}X^{B32}X^{B42}X^{B52}X^{B62}X^{B72}X^{B82} | ISYSGGRT |
|---|---|---|
| (B-1) wherein | (B-1-1) wherein | (B-1-2, SEQ ID |
| X^{B21} represents S, N, D, W, or H, | X^{B22} represents S, N, D, or H, | NO: 12) |
| X^{B31} represents Y, G, P, or S, | X^{B32} represents Y, G, or P, | INYDGDRT |
| X^{B41} represents S, D, or G, | X^{B42} represents S or D, | (B-1-3, SEQ ID |
| X^{B51} represents G, S, T, or A, | X^{B52} represents G or S, | NO: 13) |
| X^{B61} represents G, D, T, N, or R, | X^{B62} represents G, D, T, or N, | INGDSTKI |
| X^{B71} represents R, K, T, S, N, G, | X^{B72} represents R, K, T, S, G, | (B-1-4, SEQ ID |
| Y, or D, | or Y, | NO: 14) |
| x^{B81} represents T, I, or M. | X^{B82} represents T or I. | IDYSGGTT |
| | | (B-1-5, SEQ ID |
| | | NO: 15) |
| | | ISYSGGST |
| | | (B-1-6, SEQ ID |
| | | NO: 16) |
| | | ISGDSNYI |
| | | (B-1-7, SEQ ID |
| | | NO: 17) |
| | | IHPDGGST |
| | | (B-1-8, SEQ ID |
| | | NO: 18) |
| | | ISPDGGGT |
| | | (B-1-9, SEQ ID |
| | | NO: 19) |
| | | ISPSGGST |
| | | (B-1-10, SEQ ID |
| | | NO: 20) |
| | ISYSGGNT (B-1-11, SEQ ID NO: 21) | |
| | IWYDGTKM (B-1-12, SEQ ID NO: 22) | |
| | ISGDTNYI (B-1-13, SEQ ID NO: 23) | |
| IX^{B23}X^{B33}X^{B43}X^{B53}X^{B63}X^{B73} | IWX^{B34}X^{B44}X^{B54}X^{B64}X^{B74} (B-2-1) | IWYDADT |
| (B-2) wherein | wherein | (B-2-2, SEQ ID |
| X^{B23} represents S, N, D, W, or H, | X^{B34} represents Y or S, | NO: 24) |
| X^{B33} represents Y, G, P, or S, | X^{B44} represents D or G, | IWSGGRI |
| X^{B43} represents S, D, or G, | X^{B54} represents G or A, | (B-2-3, SEQ ID |
| X^{B53} represents G, S, T, or A, | X^{B64} represents R or D, | NO: 25) |
| X^{B63} represents R, K, T, S, N, G, | X^{B74} represents T or I. | |
| Y, or D, | | |
| X^{B73} represents T, I, or M. | | |

In formula (B-1), X^{B21} is preferably S, N, D, or H; X^{B31} is preferably Y, G, or P; X^{B41} is preferably S or D; X^{B51} is preferably G or S; X^{B61} is preferably G, D, T, or N; X^{B71} is preferably R, K, T, S, G, or Y; and X^{B81} is preferably is T or I.

Formula (B-1) is preferably formula (B-1-1) or one selected from the group consisting of formulas (B-1-2) to (B-1-13), more preferably one selected from the group consisting of formulas (B-1-2) to (B-1-10), and still more preferably one selected from the group consisting of formulas (B-1-2) to (B-1 9).

In formula (B-2), X^{B23} is preferably S, N, D, or W, more preferably W; X^{B33} is preferably Y, G, or S, more preferably Y or S; X^{B43} is preferably D or G; X^{B53} is preferably G, S, or A, more preferably G or A; X^{B63} is preferably R, K, T, or D, more preferably R or D; and X^{B73} is preferably T or I.

Formula (B-2) is preferably formula (B-2-1), more preferably formula (B-2-2) or (B-2-3), and still more preferably formula (B-2-3)

The heavy-chain CDR2 of the antibody or fragment thereof specifically bindable to the NTD of SARS-CoV-2 preferably contains the amino acid sequence selected from the group consisting of formulas (B-1-1) and (B-2-1) or from the group consisting of the formulas (B-1-2) to (B-1-13), (B-2-2), and (B 2-3), more preferably the amino acid sequence selected from the group consisting of formulas (B-1-2) to (B-1-10), (B-2-2) and (B-2-3); particularly more preferably the amino acid sequence selected from the group consisting of formulas (B-1-2) to (B-1-9) and (B-2-3), or an amino acid sequence having at least 90% identity to any of these amino acid sequences. The identity is preferably at least 91%, at least 92%, at least 93%, at least 94%, or at least 95%.

In an embodiment, the heavy-chain CDR3 of the antibody or fragment thereof specifically bindable to the NTD of SARS-CoV-2 preferably contains the following amino acid sequence:
the amino acid sequence represented by any of formulas (C-1) to (C-8) shown in Table 4,
an amino acid sequence having at least 90% (preferably at least 95%) identity to the amino acid sequence represented by any of formulas (C-1) to (C-8), or
an amino acid sequence formed of the amino acid sequence represented by any of formulas (C-1) to (C-8) with 1 to 3 (preferably 1 or 2, more preferably 1) amino acid mutations (preferably conservative substitution).

TABLE 4

| X^{C11}X^{C21}X^{C31}X^{C41}X^{C51}X^{C61}X^{C71}X^{C81}X^{C91}X^{C101}X^{C111} | X^{C12}RDGGSYX^{C82}SPIX^{C122}X^{C132}QX^{C152} | ARDGGSYYSPINFQF |
|---|---|---|
| X^{C121}X^{C131}X^{C141}X^{C151} (C-1) wherein | (C-1-1) wherein | (C-1-2, SEQ ID |
| X^{C11} represents A or S, | X^{C12} represents A or S, | NO: 26) |
| X^{C21} represents R, T, or K, | X^{C82} represents Y, H, or S, | SRDGGSYHSPISCQY |
| X^{C31} represents D, N, S, or Y, | X^{C122} represents N or S, | (C-1-3, SEQ ID |
| X^{C41} represents G, S, or Y, | X^{C132} represents F or C, | NO: 27) |
| X^{C51} represents G, I, or D, | X^{C152} represents F or Y. | ARDGGSYSSPINFQY |
| X^{C61} represents S or I, | | (C-1-4, SEQ ID |
| X^{C71} represents Y, V, or A, | | NO: 28) |
| X^{C81} represents Y, H, S, T, K, or E, | | SRDGGSYSSPINFQY |
| X^{C91} represents S, I, or D, | | (C-1-5, SEQ ID |
| X^{C101} represents P or Y, | | NO: 29) |
| x^{C111} represents I, Y, G, P, T, A, or V, | ARDGGSVTSPIYFQV | |
| X^{C121} represents N, S, Y, P, G, or F, | (C-1-6, SEQ ID NO: 30) | |
| X^{C131} represents F, C, M, or L, | ARDGGSVKSPIYFQY | |
| X^{C141} represents Q or D, | (C-1-7, SEQ ID NO: 31) | |
| X^{C151} represents F, Y, V, or S. | | |
| X^{C13}X^{C23}X^{C33}X^{C43}X^{C53}X^{C63}X^{C73}X^{C83} (C-2) | ATX^{C34}X^{C44}X^{C54}X^{C64}X^{C74}X^{C84} | ATNYNFQY |
| wherein | (C-2-1) wherein | (C-2-2, SEQ ID |
| X^{C13} represents A or S, | X^{C34} represents N or S, | NO: 32) |
| X^{C23} represents R, T, or K, | X^{C44} represents Y or G, | ATSGPMDV |
| X^{C33} represents D, N, S, or Y, | X^{C54} represents N or P, | (C-2-3, SEQ ID |
| X^{C43} represents I, Y, G, P, T, A, or V, | X^{C64} represents F or M, | NO: 33) |
| X^{C53} represents N, S, Y, P, G, or F, | X^{C74} represents Q or D, | |
| X^{C63} represents F, C, M, or L, | X^{C84} represents Y or V. | |
| X^{C73} represents Q or D, | | |
| X^{C83} represents F, Y, V, or S. | | |

| | |
|---|---|
| X^{C15}X^{C25}X^{C35}X^{C45}X^{C55}X^{C65}X^{C75}X^{C85}X^{C95}TIFTFX^{C155}X^{C165}X^{C175}X^{C185}X^{C195} | ARSIIAEIPTIFTFPPFDV |
| (C-3) wherein | (C-3-1, SEQ ID NO: 34) |
| x^{C15} represents A or S, | |
| x^{C25} represents R, T, or K, | |
| X^{C35} represents G, S, or Y, | |
| X^{C45} represents G, I, or D, | |
| X^{C55} represents S or I, | |
| X^{C65} represents Y, V, or A, | |
| X^{C75} represents Y, H, S, T, K, or E, | |
| X^{C85} represents S, I, or D, | |
| X^{C95} represents P or Y, | |
| X^{C155} represents I, Y, G, P, T, A, or V, | |
| X^{C165} represents N, S, Y, P, G, or F, | |
| X^{C175} represents F, C, M, or L, | |
| X^{C185} represents Q or D, | |
| X^{C195} represents F, Y, V, or S. | |
| ARLWSGYALDI (C-4, SEQ ID NO: 35) | |
| X^{C16}X^{C26}X^{C36}X^{C46}X^{C56}X^{C66}X^{C76}X^{C86}X^{C96}X^{C106}X^{C116}X^{C126}X^{C136} (C-5) | AKYYDYDYTGFDV |
| wherein | (C-5-1, SEQ ID NO: 36) |
| X^{C16} represents A or S, | |
| X^{C26} represents R, T, or K, | |
| X^{C36} represents D, N, S, or Y, | |
| X^{C46} represents G, S, or Y, | |
| X^{C56} represents G, I, or D, | |
| X^{C66} represents Y, H, S, T, K, or E, | |
| X^{C76} represents S, I, or D, | |
| X^{C86} represents P or Y, | |
| X^{C96} represents I, Y, G, P, T, A, or V, | |
| X^{C106} represents N, S, Y, P, G, or F, | |
| X^{C116} represents F, C, M, or L, | |
| X^{C126} represents Q or D, | |
| X^{C136} represents F, Y, V, or S. | |
| VX^{C27}PLLVX^{C77}HGX^{C107}X^{C117}X^{C127}X^{C137}X^{C147} (C-6) | VSPLLVVHGPFFQY |
| wherein | (C-6-1, SEQ ID NO: 37) |
| X^{C27} represents S or K, | VSPLLVYHGPFFQY |
| X^{C77} represents V, Y, or L, | (C-6-2, SEQ ID NO: 38) |
| X^{C107} represents I, Y, G, P, T, A, or V, | VKPLLVLHGPFFQS |
| X^{C117} represents N, S, Y, P, G, or F, | (C-6-3, SEQ ID NO: 39) |
| X^{C127} represents F, C, M, or L, | |
| X^{C137} represents Q or D, | |
| X^{C147} represents F, Y, V, or S. | |
| VSPSX^{C58}X^{C68}X^{C78}X^{C88}X^{C98}X^{C108} (C-7) | VSPSGAGLDV |
| wherein | (C-7-1, SEQ ID NO: 40) |
| X^{C58} represents G or A, | |
| X^{C68} represents I, Y, G, P, T, A, or V, | |
| X^{C78} represents N, S, Y, P, G, or F, | |
| X^{C88} represents F, C, M, or L, | |
| X^{C98} represents Q or D, | |
| X^{C108} represents F, Y, V, or S. | |
| SPSX^{C49}X^{C59}X^{C69}X^{C79}X^{C89}X^{C99} (C-8) | SPSAVGFDV |
| wherein | (C-8-1, SEQ ID NO: 41) |
| X^{C49} represents G or A, | |
| X^{C59} represents I, Y, G, P, T, A, or V, | |
| X^{C69} represents N, S, Y, P, G, or F, | |
| X^{C79} represents F, C, M, or L, | |
| X^{C89} represents Q or D, | |
| X^{C99} represents F, Y, V, or S. | |

In formula (C-1), X^{C21} is preferably R; X^{C31} is preferably D; X^{C41} is preferably G; X^{C51} is preferably G; X^{C61} is preferably S; X^{C70} is preferably Y or V, more preferably Y; X^{C81} is preferably Y, H, S, T, or K, more preferably Y, H, or S; X^{C91} is preferably S; X^{C101} is preferably P; X^{C111} is preferably I; X^{C121} is preferably N, S, or Y, more preferably N or S; X^{C131} is preferably F or C; X^{C141} is preferably Q; and X^{C51} is preferably F, Y or V, more preferably F or Y.

Formula (C-1) is preferably formula (C-1-1) or one selected from the group consisting of formulas (C-1-2) to (C-1-7), and more preferably one selected from the group consisting of formulas (C-1-2) to (C-1-4).

In formula (C-2), X^{C13} is preferably A; X^{C23} is preferably T; X^{C33} is preferably N or S; X^{C43} is preferably Y or G; X^{C53} is preferably N, S, or P, more preferably N or P; X^{C63} is preferably F, C, or M, more preferably F or M; X^{C73} is preferably Q or D; and X^{C83} is preferably F, Y, or V, more preferably Y or V.

Formula (C-2) is preferably formula (C-2-1), and more preferably formula (C-2-2) or (C-2-3).

In formula (C-3), X^{C15} is preferably A; X^{C25} is preferably R; X^{C35} is preferably S; X^{C45} is preferably I; X^{C55} is preferably I; X^{C65} is preferably A; X^{C75} is preferably E; X^{C85} is preferably I; X^{C95} is preferably P; X^{C155} is preferably P; X^{C165} is preferably N, S, or P, more preferably P; X^{C175} is preferably F, C, or M, more preferably F; X^{C183} is preferably Q or D, more preferably D; and X^{C195} is preferably F, Y, or V, more preferably V.

Formula (C-3) is preferably formula (C-3-1).

In formula (C-5), X^{C16} is preferably A; X^{C26} is preferably K; X^{C36} is preferably Y; X^{C46} is preferably Y; X^{C56} is preferably D; X^{C66} is preferably Y; X^{C76} is preferably D; X^{C86} is preferably Y; X^{C96} is preferably T; X^{C106} is preferably G; X^{C116} is preferably F, C, or M, more preferably F; X^{C126} is preferably Q or D, more preferably D; and X^{C136} is preferably F, Y, or V, and more preferably V.

Formula (C-5) is preferably formula (C-5-1).

In formula (C-6), X^{C107} is preferably P; X^{C117} is preferably F; X^{C127} is preferably F, C, or M, more preferably F; X^{C137} is preferably Q or D, more preferably Q, and X^{C147} is preferably Y or S.

Formula (C-6) is preferably one selected from the group consisting of formulas (C-6-1) to (C-6-3).

In formula (C-7), X^{C58} is preferably G; X^{C68} is preferably A; X^{C78} is preferably G; X^{C88} is preferably L; X^{C98} is preferably Q or D, more preferably D; and X^{C108} is preferably F, Y, or V, more preferably V.

Formula (C-7) is preferably formula (C-7-1).

In formula (C-8), X^{C49} is preferably A; X^{C59} is preferably V; X^{C69} is preferably G; X^{C79} is preferably F, C, or M, more preferably F; X^{C89} is preferably Q or D, more preferably D; and X^{C99} is preferably F, Y, or V, more preferably V.

Formula (C-8) is preferably formula (C-8-1).

The heavy-chain CDR3 of the antibody or fragment thereof specifically bindable to the NTD of SARS-CoV-2 preferably contains the amino acid sequence selected from the group consisting of formulas (C-1), (C-2), (C-4), and (C-6) to (C-8), more preferably the amino acid sequence selected from the group consisting of formulas (C-1-1), (C-2-1), and (C-6) to (C-8), the amino acid sequence selected from the group consisting of formulas (C-1-2) to (C-1-7), (C-2-2), (C-2-3), (C-4), (C-6-1) to (C-6-3), (C-7-1), and (C-8-1), and more preferably the amino acid sequence selected from the group consisting of formulas (C-1-2) to (C-1-5), (C-2-2), (C-2-3), (C-4), (C-6-1), (C-7-1), and (C-8-1), or an amino acid sequence having at least 90% identity to any of these amino acid sequences. The identity is preferably at least 91%, at least 92%, at least 93%, at least 94%, or at least 950.

In an embodiment, the light-chain CDR1 of the antibody or fragment thereof specifically bindable to the NTD of SARS-CoV-2 preferably contains the following amino acid sequence:
the amino acid sequence represented by any of formulas (D-1) to (D-3) shown in Table 5,
an amino acid sequence having at least 90% (preferably at least 95%) identity to the amino acid sequence represented by any of formulas (D-1) to (D-3), or
an amino acid sequence formed of the amino acid sequence represented by any of formulas (D-1) to (D-3) with 1 to 3 (preferably 1 or 2, more preferably 1) amino acid mutations (preferably conservative substitution).

TABLE 5

| | |
|---|---|
| SX^{D21}X^{D31}X^{D41}X^{D51}X^{D61}X^{D71} (D-1) | SEYKHYN (D-1-1, SEQ ID NO: 42) |
| wherein | SEHRSYY (D-1-2, SEQ ID NO: 43) |
| X^{D21} represents E, D, Q, or G, | SDYSHYS (D-1-3, SEQ ID NO: 44) |
| X^{D31} represents H, Y, G, or S, | SQGINKW (D-1-4, SEQ ID NO: 45) |
| X^{D41} represents R, S, I, K, or L, | SGHSSYY (D-1-5, SEQ ID NO: 46) |
| X^{D51} represents S, H, N, or F, | SEHSSYY (D-1-6, SEQ ID NO: 47) |
| X^{D61} represents Y or K, | SDHSSYY (D-1-7, SEQ ID NO: 48) |
| X^{D71} represents Y, S, W, or N. | |
| SX^{D23}X^{D33}X^{D43}X^{D53}X^{D63}X^{D73}GKNKDL (D-2) | SQSLFYSGKNKDL (D-2-1, SEQ ID NO: 49) |
| wherein | |
| X^{D23} represents E, D, Q, or G, | |
| X^{D33} represents H, Y, G, or S, | |
| X^{D43} represents R, S, I, K, or L, | |
| X^{D53} represents S, H, N, or F, | |
| X^{D63} represents Y or K, | |
| X^{D73} represents Y, S, W, or N. | |

NIGGKT (D-3, SEQ ID NO: 50)

Formula (D-1) is preferably one selected from the group consisting of formulas (D-1-1) to (D-1-7).

In formula (D-2), X^{D23} is preferably Q; XD 33 is preferably S; X^{D43} is preferably L; X^{D53} is preferably F; X^{D63} is preferably Y or K, more preferably Y; and X^{D73} is preferably Y, S, or W, more preferably S.

Formula (D-2) is preferably formula (D-2-1).

The light-chain CDR1 of the antibody or fragment thereof specifically bindable to the NTD of SARS-CoV-2 preferably contains the amino acid sequence of formula (D-1), more preferably the amino acid sequence selected from the group consisting of formulas (D-1-1) to (D-1-7), or an amino acid sequence having at least 90% identity to any of these amino acid sequences. The identity is preferably at least 91%, at least 92%, at least 93%, at least 94%, or at least 95%.

In an embodiment, the light-chain CDR2 of the antibody or fragment thereof specifically bindable to the NTD of SARS-CoV-2 preferably contains the following amino acid sequence:
the amino acid sequence represented by any of formulas (E-1) to (E-3) shown in Table 6,
an amino acid sequence having at least 90% (preferably at least 95%) identity to the amino acid sequence represented by any of formulas (E-1) to (E-3), or
an amino acid sequence formed of the amino acid sequence represented by any of formulas (E-1) to (E-3) with 1 to 3 (preferably 1 or 2, more preferably 1) amino acid mutations (preferably conservative substitution).

TABLE 6

| | | |
|---|---|---|
| X^{E11}X^{E21}SX^{E41}GSX^{E71} (E-1) | X^{E12}X^{E22}SDGSX^{E72} (E-1-1) | LKSDGSH (E-1-2, SEQ ID NO: 51) |
| wherein | wherein | VKSDGSH (E-1-3, SEQ ID NO: 52) |
| X^{E11} represents L, V, or I, | X^{E12} represents L, V, or I, | IESDGSH (E-1-4, SEQ ID NO: 53) |
| X^{E21} represents K, N, or E, | X^{E22} represents K, or E, | VKSDGSL (E-1-5, SEQ ID NO: 54) |
| X^{E41} represents D, Y, or K, | X^{E72} represents H or L. | |

| | |
|---|---|
| X^{E71} represents H or L. | LNSYGSH (E-1-6, SEQ ID NO: 55) |
| | LNSKGSH (E-1-7, SEQ ID NO: 56) |
| | IESKGSH (E-1-8, SEQ ID NO: 57) |

VGTSDIVG (E-2, SEQ ID NO: 58)

| | |
|---|---|
| X^{E13}X^{E23}X^{E33} (E-3) | KAN (E-3-1) |
| wherein | SHN (E-3-2) |
| X^{E13} represents K, S, or W, | WAS (E-3-3) |
| X^{E23} represents A or H, | |
| X^{E33} represents N or S. | |

In formula (E-1), X^{E21} is preferably K or E, and X^{E41} is preferably D.

Formula (E-1) is preferably formula (E-1-1) or one selected from the group consisting of formulas (E-1-2) to (E-1-8), and more preferably one selected from the group consisting of formulas (E-1-2) to (E-1-5).

In formula (E-3), X^{E13} is preferably K, X^{E23} is preferably A, and X^{E33} is preferably N.

Formula (E-3) is preferably one selected from the group consisting of formulas (E-3-1) to (E-3-3), and more preferably formula (E-3-1)

The light-chain CDR2 of the antibody or fragment thereof specifically bindable to the NTD of SARS-CoV-2 preferably contains the amino acid sequence selected from the group consisting of formulas (E-1-1), (E-2), and (E-3), or the amino acid sequence selected from the group consisting of formulas (E 1-2) to (E-1-8), (E-2), and (E-3-1) to (E-3-3); more preferably the amino acid sequence selected from the group consisting of formulas (E-1-2) to (E-1-5), (E-2), and (E-3-1); or an amino acid sequence having at least 90% identity to any of these amino acid sequences. The identity is preferably at least 91%, at least 92%, at least 93%, at least 94%, or at least 95%.

In an embodiment, the light-chain CDR3 of the antibody or fragment thereof specifically bindable to the NTD of SARS-CoV-2 preferably contains the following amino acid sequence:
the amino acid sequence represented by any of formulas (F-1) to (F-7) shown in Table 7,
an amino acid sequence having at least 90% (preferably at least 95%) identity to the amino acid sequence represented by any of formulas (F-1) to (F-7), or
an amino acid sequence formed of the amino acid sequence represented by any of formulas (F-1) to (F-7) with 1 to 3 (preferably 1 or 2, more preferably 1) amino acid mutations (preferably conservative substitution).

TABLE 7

| GX^{F21}X^{F31}X^{F41}X^{F51}X^{F61}X^{F71}X^{F81}X^{F91}X^{F101} | GVSX^{F42}SGGHX^{F92}X^{F102} (F-1-1) | GVSYSGGHNI |
|---|---|---|
| (F-1) wherein | wherein | (F-1-2, SEQ ID NO: 59) |
| X^{F21} represents V or A, | X^{F42} represents Y or F, | GVSFSGGHGV |
| X^{F31} represents S, D, N, G, or K, | X^{F92} represents N or G, | (F-1-3, SEQ ID NO: 60) |
| X^{F41} represents Y, F, V, L, or H, | X^{F102} represents I or V. | GVSYSGGHNV |
| X^{F51} represents S, K, or N, | | (F-1-4, SEQ ID NO: 61) |
| X^{F61} represents G or N, | GVSVSGGHNL | |
| X^{F71} represents G, A, E, or K, | (F-1-5, SEQ ID NO: 62) | |
| X^{F81} represents H, S, Y, or V, | GVSLKGGHNL | |
| X^{F91} represents N, G, or S, | (F-1-6, SEQ ID NO: 63) | |
| X^{F101} represents I, V, L, or Y. | | |
| GX^{F23}X^{F33}X^{F43}X^{F53}X^{F63}X^{F73}X^{F83}X^{F93}YX^{F113} | GADYSGASSYV | |
| (F-2) wherein | (F-2-1, SEQ ID NO: 64) | |
| X^{F23} represents V or A, | | |
| X^{F33} represents S, D, N, G, or K, | | |
| X^{F43} represents Y, F, V, L, or H, | | |
| X^{F53} represents S, K, or N, | | |
| X^{F63} represents G or N, | | |
| X^{F73} represents G, A, E, or K, | | |
| X^{F83} represents H, S, Y, or V, | | |
| X^{F93} represents N, G, or S, | | |
| X^{F113} represents I, V, L, or Y. | | |
| GX^{F24}X^{F34}X^{F44}X^{F54}TX^{F74}X^{F84}X^{F94}YX^{F114}YX^{F134} | GANHNIGESYGYV | |
| (F-3) wherein | (F-3-1, SEQ ID NO: 65) | |
| X^{F24} represents V or A, | | |
| X^{F34} represents S, D, N, G, or K | | |
| X^{F44} represents Y, F, V, L, or H, | | |
| X^{F54} represents S, K, or N, | | |
| X^{F74} represents G or N, | | |
| X^{F84} represents G, A, E, or K, | | |
| X^{F94} represents H, S, Y, or V, | | |
| X^{F114} represents N, G, or S, | | |
| X^{F134} represents I, V, L, or Y. | | |
| GX^{F25}X^{F35}X^{F45}X^{F55}X^{F65}X^{F75}X^{F85}X^{F95} (F-4) | GVX^{F36}YX^{F56}X^{F66}X^{F76}X^{F86}X^{F96} | GVGYNNKYV |
| wherein | (F-4-1) wherein | (F-4-2, SEQ ID NO: 66) |
| X^{F25} represents V or A, | X^{F36} represents S, G, or K, | GVGYSGGYV |
| X^{F35} represents S, D, N, G, or K, | X^{F56} represents S or N, | (F-4-3, SEQ ID NO: 67) |
| X^{F45} represents Y, F, V, L, or H, | X^{F66} represents G or N, | GVSYSGGYV |
| X^{F55} represents S, K, or N, | X^{F76} represents G or K, | (F-4-4, SEQ ID NO: 68) |
| X^{F65} represents G or N, | X^{F86} represents Y or V, | GVKYSGGVV |
| X^{F75} represents G, A, E, or K, | X^{F96} represents V or Y. | (F-4-5, SEQ ID NO: 69) |
| X^{F85} represents H, S, Y, or V, | | GVSYSGGYY |
| X^{F95} represents I, V, L, or Y. | | (F-4-6, SEQ ID NO: 70) |

QYDSTPPLT (F-5, SEQ ID NO: 71)
QVWDSYTYV (F-6, SEQ ID NO: 72)
QFINGPLT (F-7, SEQ ID NO: 73)

In formula (F-1), X^{F21} is preferably V; X^{F31} is preferably S; X^{F41} is preferably Y, F, V, or L, more preferably Y or F; X^{F51} is preferably S or K, more preferably S; X^{F61} is preferably G; X^{F71} is preferably G; X^{F81} is preferably H; X^{F91} is preferably N or G; and X^{F101} is preferably I, V, or L, more preferably I or V.

Formula (F-1) is preferably formula (F-1-1) or one selected from the group consisting of formulas (F-1-2) to (F-1-6), and more preferably one selected from the group consisting of formulas (F-1-2) to (F-1-4).

In formula (F-2), X^{F23} is preferably A; X^{F33} is preferably S or D, more preferably D; X^{F43} is preferably Y, F, V, or L, more preferably Y or F, still more preferably Y; X^{F53} is preferably S or K, more preferably S; X^{F63} is preferably G; X^{F73} is preferably G or A, more preferably A; X^{F83} is preferably H or S, preferably S; X^{F93} is preferably S; and X^{F113} is preferably I, V, or L, more preferably I or V, and still more preferably V.

Formula (F-2) is preferably formula (F-2-1).

In formula (F-3), X^{F24} is preferably A; X^{F34} is preferably S, D, or N, more preferably N; X^{F44} is preferably Y, F, or H, more preferably H; X^{F54} is preferably S or N, more preferably N; X^{F74} is preferably G; X^{F84} is preferably G or E, more preferably E; X^{F94} is preferably H or S, preferably S; X^{F114} is preferably G; and X^{F134} is preferably I, V, or L, more preferably I or V, still more preferably V.

Formula (F-3) is preferably formula (F-3-1).

In formula (F-4), X^{F25} is preferably V; X^{F35} is preferably S, G, or K; X^{F45} is preferably Y; X^{F55} is preferably S or N; X^{F65} is preferably G or N; X^{F75} is preferably G or K; X^{F85} is preferably Y or V; and X^{F95} is preferably V or Y.

Formula (F-4) is preferably formula (F-4-1), more preferably one selected from the group consisting of formulas (F 4-2) to (F-4-6), and still more preferably one selected from the group consisting of formulas (F-4-2) to (F-4-4).

The light-chain CDR3 of the antibody or fragment thereof specifically bindable to the NTD of SARS-CoV-2 preferably contains the amino acid sequence selected from the group consisting of formulas (F-1) to (F-5); more preferably the amino acid sequence selected from the group consisting of formulas (F 1-1), (F-2-1), (F-3-1), (F-4-1), and (F-5); still more preferably the amino acid sequence selected from the group consisting of formulas (F-1-2) to (F-1-6), (F-2-1), (F-3-1), (F-4-2) to (F-4-6), and (F-5); and particularly more preferably the amino acid sequence selected from the group consisting of formulas (F-1-2) to (F-1-4), (F-2-1), (F-3-1), (F-4-2) to (F-4-4), and (F-5); or an amino acid sequence having at least 90% identity to any of these amino acid sequences. The identity is preferably at least 91%, at least 92%, at least 93%, at least 94%, or at least 95%.

Table 8 shows preferable examples of combinations of the heavy-chain CDR1 to CDR3 and the light-chain CDR1 to CDR3 of the antibody or fragment thereof specifically bindable to the NTD of SARS-CoV-2.

TABLE 8

| | Heavy-chain | Heavy-chain | Heavy-chain | Light-chain | Light-chain | Light-chain |
|---|---|---|---|---|---|---|
| Combination | CDR1 | CDR2 | CDR3 | CDR1 | CDR2 | CDR3 |
| N-1 | (A-1) | (B-1) or | (C-1), | (D-1) | (E-1), | (F-1), |
| | | (B-2) | (C-2), | | (E-2), or | (F-2), |
| | | | (C-4), | | (E-3) | (F-3), |
| | | | (C-6), | | | (F-4), or |
| | | | (C-7), or | | | (F-5) |
| | | | (C-8) | | | |
| N-2 | (A-1-1) | (B-1-1) or | (C-1-1), | (D-1-1) | (E-1-1), | (F-1-1), |
| | | (B-2-1) | (C-2-1), | | (E-2), or | (F-2-1), |
| | | | (C-4), | | (E-3) | (F-3), |
| | | | (C-6), | | | (F-4-1), or |
| | | | (C-7), or | | | (F-5) |
| | | | (C-8) | | | |
| N-3 | (A-1-2), | (B-1-2), | (C-1-2), | (D-1-1), | (E-1-2), | (F-1-2), |
| | (A-1-3), | (B-1-3), | (C-1-3), | (D-1-2), | (E-1-3), | (F-1-3), |
| | (A-1-4), | (B-1-4), | (C-1-4), | (D-1-3), | (E-1-4), | (F-1-4), |
| | (A-1-5), | (B-1-5), | (C-1-5), | (D-1-4), | (E-1-5), | (F-2-1), |
| | (A-1-6), | (B-1-6), | (C-2-2), | (D-1-5), | (E-2), or | (F-3-1), |
| | (A-1-7), or | (B-1-7), | (C-2-3), | (D-1-6), or | (E-3-1) | (F-4-2), |
| | (A-1-8) | (B-1-8), | (C-4), | (D-1-7) | | (F-4-3), |
| | | (B-1-9), or | (C-6-1), | | | (F-4-4), or |
| | | (B-2-3) | (C-7-1), or | | | (F-5) |
| | | | (C-8-1) | | | |
| N-4 | (A-1-2) | (B-1-2) | (C-1-2) | (D-1-2) | (E-1-2) | (F-1-2) |
| N-5 | (A-1-3) | (B-1-3) | (C-2-2) | (D-1-3) | (E-2) | (F-2-1) |
| N-6 | (A-1-4) | (B-1-4) | (C-2-3) | (D-1-4) | (E-3-1) | (F-5) |
| N-7 | (A-1-5) | (B-1-5) | (C-1-3) | (D-1-5) | (E-1-3) | (F-1-3) |
| N-8 | (A-1-8) | (B-2-3) | (C-4) | (D-1-1) | (E-1-5) | (F-3-1) |
| N-9 | (A-1-2) | (B-1-6) | (C-1-4) | (D-1-6) | (E-1-2) | (F-1-4) |
| N-10 | (A-1-7) | (B-1-8) | (C-7-1) | (D-1-6) | (E-1-2) | (F-4-2) |
| N-11 | (A-1-7) | (B-1-9) | (C-8-1) | (D-1-7) | (E-1-2) | (F-4-3) |
| N-12 | (A-1-6) | (B-1-7) | (C-6-1) | (D-1-6) | (E-1-4) | (F-4-4) |
| N-13 | (A-1-2) | (B-1-6) | (C-1-5) | (D-1-6) | (E-1-3) | (F-1-4) |
| N-14 | (A-1-2), | (B-1-2), | (C-1-2), | (D-1-1), | (E-1-2), | (F-1-2), |
| | (A-1-3), | (B-1-3), | (C-1-3), | (D-1-2), | (E-1-3), | (F-1-3), |
| | (A-1-4), | (B-1-4), | (C-1-4), | (D-1-3), | (E-1-4), | (F-1-4), |
| | (A-1-5), | (B-1-5), | (C-1-5), | (D-1-4), | (E-1-5), | (F-1-5), |
| | (A-1-6), | (B-1-6), | (C-1-6), | (D-1-5), | (E-1-6), | (F-1-6), |
| | (A-1-7), | (B-1-7), | (C-1-7), | (D-1-6), | (E-1-7), | (F-2-1), |
| | (A-1-8), | (B-1-8), | (C-2-2), | (D-1-7), | (E-1-8), | (F-3-1), |
| | (A-1-9), or | (B-1-9), | (C-2-3), | (D-2-1), or | (E-2), | (F-4-2), |
| | (A-2-2) | (B-1-10), | (C-3-1), | (D-3) | (E-3-1), | (F-4-3), |
| | | (B-1-11), | (C-4), | | (E-3-2), or | (F-4-4), |
| | | (B-1-12), | (C-5-1), | | (E-3-3) | (F-4-5), |
| | | (B-1-13), | (C-6-1), | | | (F-4-6), |
| | | (B-2-2), or | (C-6-2), | | | (F-5), |
| | | (B-2-3) | (C-6-3), | | | (F-6), or |
| | | | (C-7-1), or | | | (F-7) |
| | | | (C-8-1) | | | |

It is also preferred that in combinations N-3 to N-14, one to three amino acids are mutated (preferably conservatively substituted) in the amino acid sequence of at least one CDR of the heavy-chain CDR1 to CDR3 and the light-chain CDR1 to CDR3.

### 3. Antibody or Fragment Thereof Specifically Bindable to the CTD of the N Protein of SARS-CoV-2

The CTD (simply “CTD”) of the N protein of SARS-CoV-2 means the amino acid region at positions 247 to 419 from the N-terminus of the N protein and has the following amino acid sequence:

(SEQ ID NO: 2)
TKKSAAEASKKPRQKRTATKAYNVTQAFGRRGPEQTQGNFGDQELIRQGT
DYKHWPQIAQFAPSASAFFGMSRIGMEVTPSGTWLTYTAAIKLDDKDPNF
KDQVILLNKHIDAYKTFPPTEPKKDKKKKADETQALPQRQKKQQTVTLLP
AADLDDFSKQLQQSMSSADSTQA

The antibody or fragment thereof specifically bindable to the CTD of SARS-CoV-2 preferably does not bind to the CTD of SARS-CoV or has low bindability to the CTD of SARS-CoV. In the amino acid sequence of SEQ ID NO: 2, for example, the 267th A, 290th E, 334th T, 345th N, and 349th Q from the N-terminus of the N protein differ from their corresponding amino acid in the CTD of SARS-CoV. The antibody or fragment thereof against the N protein of SARS-CoV-2 (in particular, the antibody or fragment thereof specifically bindable to the CTD of SARS-CoV-2) preferably binds to a region (or an epitope) containing one or more amino acids selected from the group consisting of the 267th A, 290th E, 334th T, 345th N, and 349th Q from the N-terminus of the N protein in the amino acid sequence of SEQ ID NO: 2.

In an embodiment, the antibody or fragment thereof against the N protein of SARS-CoV-2 (in particular, the antibody or fragment thereof specifically bindable to the CTD of SARS-CoV-2) may bind to the amino acid region at positions 247 to 419 from the N-terminus of the N protein, preferably to the dimeric domain of the CTD, or preferably to the amino acid region at positions 247 to 364 or 256 to 364 from the N-terminus of the N protein.

In an embodiment, the antibody or fragment thereof against the N protein of SARS-CoV-2 (in particular, the antibody or fragment thereof specifically bindable to the CTD of SARS-CoV-2) preferably binds to the amino acid region at positions 250 to 257, 374 to 382, or 394 to 405 from the N terminus of the N protein; i.e., it is preferred that the epitope is present in the region composed of any of the amino acid sequences of SEQ ID Nos: 150 to 152 on the N protein.

The antibody or fragment thereof specifically bindable to the CTD of SARS-CoV-2 can be produced according to known methods in combination, such as by a method including steps (P1) to (P5) and the following step (P6′):
(P6′) obtaining antibodies that specifically bind to the CTD from the antibodies recovered in step (P5).

Step (P6′) can be performed in the same manner as step (P6) by selecting antibodies that specifically bind to the CTD according to a known screening method, such as enzyme-linked immunosorbent assay (ELISA), western blotting, or immunostaining.

The method including steps (P1) to (P5) and (P6′) can provide multiple types of antibodies or fragments thereof that specifically bind to the CTD of SARS-CoV-2. (The antibodies or fragments thereof differ from each other in terms of the amino acid sequence of at least one CDR of the heavy-chain CDR1 to CDR3 and the light-chain CDR1 to CDR3.)

In an embodiment, the heavy-chain CDR1 of the antibody or fragment thereof specifically bindable to the CTD of SARS-CoV-2 preferably contains the following amino acid sequence:
the amino acid sequence represented by either formula (G-1) or (G-2) shown in Table 9,
an amino acid sequence having at least 90% (preferably at least 95%) identity to the amino acid sequence represented by formula (G-1) or (G-2), or
an amino acid sequence formed of the amino acid sequence represented by either formula (G-1) or (G-2) with 1 to 3 (preferably 1 or 2, more preferably 1) amino acid mutations (preferably conservative substitution).

TABLE 9

| | | |
|---|---|---|
| GX^{G21}X^{G31}X^{G41}X^{G51}X^{G61}X^{G71}X^{G81} (G-1) | GX^{G22}X^{G32}X^{G42}X^{G52}X^{G62}X^{G72}X^{G82} | GFIFSNYF |
| wherein | (G-1-1) wherein | (G-1-2, SEQ ID NO: 74) |
| X^{G21} represents F or L, | X^{G22} represents F or L, | GFTFNTYT |
| X^{G31} represents I, T, S, or F, | X^{G32} represents I, T, or S, | (G-1-3, SEQ ID NO: 75) |
| X^{G41} represents F or L, | X^{G42} represents F or L, | GFTFSNYY |
| X^{G51} represents S, N, T, R, or D, | X^{G52} represents S, N, T, or R, | (G-1-4, SEQ ID NO: 76) |
| X^{G61} represents N, T, or D, | X^{G62} represents N or T, | GLSLTNYT |
| X^{G71} represents Y, S, or H, | X^{G72} represents Y, S, or H, | (G-1-5, SEQ ID NO: 77) |
| X^{G81} represents F, T, Y, W, or G. | X^{G82} represents F, T, Y, or W. | GFSFSNSW |
| | | (G-1-6, SEQ ID NO: 78) |
| | | GFTFSNYW |
| | | (G-1-7, SEQ ID NO: 79) |
| | | GFTFRNHW |
| | | (G-1-8, SEQ ID NO: 80) |
| | | GFTFSNHW |
| | | (G-1-9, SEQ ID NO: 81) |

GFFFSNSW (G-1-10, SEQ ID NO: 82)
GFTFDDYG (G-1-11, SEQ ID NO: 83)

KLSERY (G-2, SEQ ID NO: 84)

In formula (G-1), X^{G31} is preferably I, T, or S; X^{G51} is preferably S, N, T, or R; X^{G61} is preferably N or T; X^{G71} is preferably Y, S, or H; and X^{G81} is preferably F, T, Y, or W.

Formula (G-1) is preferably formula (G-1-1), more preferably one selected from the group consisting of formulas (G 1-2) to (G-1-11), and still more preferably one selected from the group consisting of formulas (G-1-2) to (G-1-9).

The heavy-chain CDR1 of the antibody or fragment thereof specifically bindable to the CTD of SARS-CoV-2 preferably contains the amino acid sequence of formula (G-1-1), more preferably the amino acid sequence selected from the group consisting of formulas (G-1-2) to (G-1-11), still more preferably the amino acid sequence selected from the group consisting of formulas (G-1-2) to (G-1-9), or an amino acid sequence having at least 90% identity to any of these amino acid sequences. The identity is preferably at least 91%, at least 92%, at least 93%, at least 94%, or at least 95%.

In an embodiment, the heavy-chain CDR2 of the antibody or fragment thereof specifically bindable to the CTD of SARS-CoV-2 preferably contains the following amino acid sequence:
the amino acid sequence represented by any of formulas (H-1) to (H-3) shown in Table 10,
an amino acid sequence having at least 90% (preferably at least 95%) identity to the amino acid sequence represented by any of formulas (H-1) to (H-3), or
an amino acid sequence formed of the amino acid sequence represented by any of formulas (H-1) to (H-3) with 1 to 3 (preferably 1 or 2, more preferably 1) amino acid mutations (preferably conservative substitution).

TABLE 10

| | | |
|---|---|---|
| IX^{H21}X^{H31}X^{H41}X^{H51}X^{H61}X^{H71}X^{H81} (H-1) | IX^{H22}X^{H32}X^{H42}X^{H52}X^{H62}X^{H72}X^{H82} | ISGDSTYI |
| wherein | (H-1-1) wherein | (H-1-2, SEQ ID NO: 85) |
| X^{H21} represents S, G, K, or N, | X^{H22} represents S, G, K, or N, | IGNSGSKT |
| X^{H31} represents G, N, S, Y, T, or P, | X^{H32} represents G, N, S, T, or P, | (H-1-3, SEQ ID NO: 86) |
| X^{H41} represents D, S, T, or A, | X^{H42} represents D, S, or A, | ISGDGSNI |
| X^{H51} represents S, G, or D, | X^{H52} represents S, G, or D, | (H-1-4, SEQ ID NO: 87) |
| X^{H61} represents T, S, A, G, or R, | X^{H62} represents T, S, G, or R, | ISGDSSNI |
| X^{H71} represents Y, K, N, T, I, or S, | X^{H72} represents Y, K, N, T, I, or S, | (H-1-5, SEQ ID NO: 88) |
| X^{H81} represents I, T, P, or V. | X^{H82} represents I, T, P, or V. | IKGDSSII |
| | | (H-1-6, SEQ ID NO: 89) |
| | | INPDDGSP |
| | | (H-1-7, SEQ ID NO: 90) |
| | | IKGDSSIT |
| | | (H-1-8, SEQ ID NO: 91) |
| | | INSDGSNT |
| | | (H-1-9, SEQ ID NO: 92) |
| | | INTDGSST |
| | | (H-1-10, SEQ ID NO: 93) |
| | | IKGDSRTI |
| | | (H-1-11, SEQ ID NO: 94) |
| | | ISGASSNI |
| | | (H-1-12, SEQ ID NO: 95) |
| | | ISGDSSNV |
| | | (H-1-13, SEQ ID NO: 96) |
| | | IKGDSSTI |
| | | (H-1-14, SEQ ID NO: 97) |
| | | INSDGSST |
| | | (H-1-15, SEQ ID NO: 98) |

IGYSGASI (H-1-16, SEQ ID NO: 99)
ISGTSSNI (H-1-17, SEQ ID NO: 100)

MWSDGDT (H-2, SEQ ID NO: 101)
NDS (H-3)

In formula (H-1), X^{H31} is preferably G, N, S, T, or P; X^{H41} is preferably D, S, or A; and X^{H61} is preferably T, S, G, or R.

Formula (H-1) is preferably formula (H-1-1) or one selected from the group consisting of formulas (H-1-2) to (H-1-17), more preferably one selected from the group consisting of formulas (H-1-2) to (H-1-15), and still more preferably one selected from the group consisting of formulas (H-1-2) to (H-1-7) and (H-1-9) to (H-1-12)

The heavy-chain CDR2 of the antibody or fragment thereof specifically bindable to the CTD of SARS-CoV-2 preferably contains the amino acid sequence selected from the group consisting of formulas (H-1) and (H-2), more preferably the amino acid sequence selected from the group consisting of formulas (H 1-1) and (H-2), or from the group consisting of formulas (H-1-2) to (H-1-17) and (H-2), still more preferably the amino acid sequence selected from the group consisting of formulas (H-1-2) to (H-1-15) and (H-2), and particularly preferably the amino acid sequence selected from the group consisting of formulas (H-1-2) to (H-1-7), (H-1-9) to (H-1-12), and (H-2), or an amino acid sequence having at least 90% identity to any of these amino acid sequences. The identity is preferably at least 910, at least 92%, at least 93%, at least 94%, or at least 95%.

In an embodiment, the heavy-chain CDR3 of the antibody or fragment thereof specifically bindable to the CTD of SARS-CoV-2 preferably contains the following amino acid sequence:
the amino acid sequence represented by any of formulas (I-1) to (I-11) shown in Table 11,
an amino acid sequence having at least 90% (preferably at least 95%) identity to the amino acid sequence represented by any of formulas (I-1) to (I-11), or
an amino acid sequence formed of the amino acid sequence represented by any of formulas (I-1) to (I-11) with 1 to 3 (preferably 1 or 2, more preferably 1) amino acid mutations (preferably conservative substitution).

TABLE 11

| | | |
|---|---|---|
| X^{I11}X^{I21}X^{I31}X^{I41}X^{I51}X^{I61}X^{I71} (I-1) | X^{I12}X^{I22}X^{I32}X^{I42}X^{I52}X^{I62}X^{I72} (I-1-1) | TINWGDV (I-1-2, |
| wherein | wherein | SEQ ID NO: 102) |
| X^{I11} represents S or T, | X^{I12} represents S or T, | STLGVGV (I-1-3, |
| X^{I21} represents I or T, | X^{I22} represents I or T, | SEQ ID NO: 103) |
| X^{I31} represents N, L, D, or T, | X^{I32} represents N, L, or T, | TTLGVGV (I-1-4, |
| X^{I41} represents W or G, | X^{I42} represents W or G, | SEQ ID NO: 104) |
| X^{I51} represents G, V, F, or N, | X^{I52} represents G, V, F, or N, | STTGFDV (I-1-5, |
| X^{I61} represents D, G, F, or N, | X^{I62} represents D, F, or G, | SEQ ID NO: 105) |
| X^{I71} represents V or L. | X^{I72} represents V or L. | STLGNFL (I-1-6, |
| | | SEQ ID NO: 106) |
| | | TITWGDV (I-1-7, |
| | | SEQ ID NO: 107) |

TIDWGNV (I-1-8, SEQ ID NO: 108)

ARTSRIVADLVTMGYALDF (I-2, SEQ ID NO: 109)
ARDVVKTGTTGLPFDL (I-3, SEQ ID NO: 110)
AREGVITVGTWGDV (I-4, SEQ ID NO: 111)
ATSEY (I-5, SEQ ID NO: 112)
TTATDV (I-6, SEQ ID NO: 113)
TPATDV (I-7, SEQ ID NO: 114)
SPMTIHGLDT (I-8, SEQ ID NO: 115)
GSGWV (I-9, SEQ ID NO: 116)
GSGWY (I-10, SEQ ID NO: 117)
LSSESDDARV (I-11, SEQ ID NO: 118)

In formula (I-1), X^{I31} is preferably N, L, or T, and X^{I61} is preferably D, F, or G.

Formula (I-1) is preferably formula (I-1-1) or one selected from the group consisting of formulas (I-1-2) to (I-1-8), more preferably one selected from the group consisting of formulas (I-1-2) to (I-1-7), and still more preferably one selected from the group consisting of formulas (I-1-2) to (I-1-6).

The heavy-chain CDR3 of the antibody or fragment thereof specifically bindable to the CTD of SARS-CoV-2 preferably contains the amino acid sequence selected from the group consisting of formulas (I-1) to (I-3) and (I-5) to (I-9), more preferably the amino acid sequence selected from the group consisting of formulas (I-1-1), (I-2), (I-3), and (I-5) to (I-9), or the amino acid sequence selected from the group consisting of formulas (I-1-2) to (1-1-8), (1-2), (1-3), and (I-5) to (1-9), still more preferably the amino acid sequence selected from the group consisting of formulas (I-1-2) to (I-1-7), (I-2), (I-3), and (I-5) to (I-9), particularly preferably the amino acid sequence selected from the group consisting of formulas (I-1-2) to (I-1-6), (I-2), (I-3), and (I-5) to (I-9), or an amino acid sequence having at least 90% identity to any of these amino acid sequences. The identity is preferably at least 91%, at least 92%, at least 93%, at least 94%, or at least 95%.

In an embodiment, the light-chain CDR1 of the antibody or fragment thereof specifically bindable to the CTD of SARS-CoV-2 preferably contains the following amino acid sequence:
the amino acid sequence represented by any of formulas (J-1) to (J-3) shown in Table 12,
an amino acid sequence having at least 90% (preferably at least 95%) identity to the amino acid sequence represented by any of formulas (J-1) to (J-3), or
an amino acid sequence formed of the amino acid sequence represented by any of formulas (J-1) to (J-3) with 1 to 3 (preferably 1 or 2, more preferably 1) amino acid mutations (preferably conservative substitution).

TABLE 12

| X^{J11}LX^{J31}X^{J41}X^{J51}Y (J-1) | KLSX^{J42}X^{J52}Y (J-1-1) | KLSNQY |
|---|---|---|
| wherein | wherein | (J-1-2, SEQ ID NO: 119) |
| X^{J11} represents K or A, | X^{J42} represents N, E, or K, | KLSERY |
| X^{J31} represents S or P, | X^{J52} represents Q, R, or E. | (J-1-3, SEQ ID NO: 120) |
| X^{J41} represents N, E, K, T, or D, | | KLSKEY |
| X^{J51} represents Q, R, K, or E. | | (J-1-4, SEQ ID NO: 121) |
| | ALPKKY (J-1-5, SEQ ID NO: 122) | |
| | KLSTQY (J-1-6, SEQ ID NO: 123) | |
| | KLSDQY (J-1-7, SEQ ID NO: 124) | |
| SX^{J23}SLX^{J53}X^{J63}X^{J73}DGX^{J103}TX^{J123} | SX^{J24}SLX^{J54}X^{J64}SDGX^{J104}TX^{J124} | SESLLHSDGNTY |
| (J-2), wherein | (J-2-1) wherein | (J-2-2, SEQ ID NO: 125) |
| X^{J23} represents E or Q, | X^{J24} represents E or Q, | SQSLLHSDGKTY |
| X^{J53} represents L or V, | X^{J54} represents L or V, | (J-2-3, SEQ ID NO: 126) |
| X^{J63} represents H, D, or Y, | X^{J64} represents H, D, or Y, | SQSLVHSDGKTY |
| X^{J73} represents S or G, | X^{J104} represents N, K, or Y, | (J-2-4, SEQ ID NO: 127) |
| X^{J103} represents N, K, or Y, | X^{J124} represents Y or F. | SQSLLDSDGYTY |
| X^{J123} represents Y or F. | | (J-2-5, SEQ ID NO: 128) |
| | | SQSLLYSDGYTY |
| | | (J-2-6, SEQ ID NO: 129) |
| | | SQSLLYSDGYTF |
| | | (J-2-7, SEQ ID NO: 130) |
| | | SQSLLHSDGNTY |
| | | (J-2-8, SEQ ID NO: 131) |

SQSLLHGDGKTY (J-2-9, SEQ ID NO: 132)

NIGGKA (J-3, SEQ ID NO: 133)

In formula (J-1), X^{J11} is preferably K; X^{J31} is preferably S; X^{J41} is preferably N, E, or K; and X^{J51} is preferably Q, R, or E.

Formula (J-1) is preferably formula (J-1-1) or one selected from the group consisting of formulas (J-1-2) to (I-1-7), and more preferably one selected from the group consisting of formulas (J-1-2) to (J-1-4).

In formula (J-2), X^{J73} is preferably S.

Formula (J-2) is preferably formula (J-2-1) or one selected from the group consisting of formulas (J-2-2) to (J-2-9), more preferably one selected from the group consisting of formulas (J-2-2) to (J-2-8), and still more preferably one selected from the group consisting of formulas (J-2-2) to (J-2-6).

The heavy-chain CDR3 of the antibody or fragment thereof specifically bindable to the CTD of SARS-CoV-2 preferably contains the amino acid sequence selected from the group consisting of formulas (J-1-1), (J-2-1), and (J-3), or the amino acid sequence selected from the group consisting of formulas (J 1-2) to (J-1-7), (J-2-2) to (J-2-9), and (J-3), more preferably, the amino acid sequences selected from the group consisting of formulas (J-1-2) to (J-1-4), (J-2-2) to (J-2-6), and (J-3), or an amino acid sequence having at least 90% identity to any of these amino acid sequences. The identity is preferably at least 91%, at least 92%, at least 93%, at least 94%, or at least 95%.

In an embodiment, the light-chain CDR2 of the antibody or fragment thereof specifically bindable to the CTD of SARS-CoV-2 preferably contains the following amino acid sequence:
the amino acid sequence represented by formula (K-1) shown in Table 13,
an amino acid sequence having at least 90% (preferably at least 95%) identity to the amino acid sequence represented by formula (K-1), or
an amino acid sequence formed of the amino acid sequence represented by formula (K-1) with 1 to 3 (preferably 1 or 2, more preferably 1) amino acid mutations (preferably conservative substitution)

TABLE 13

| | |
|---|---|
| | NDD (K-1-1) |
| X^{K11}X^{K21}X^{K31} (K-1) | KDS (K-1-2) |
| wherein | NGN (K-1-3) |
| X^{K11} represents K, N, L, or E, | NDS (K-1-4) |
| X^{K21} represents D, G, V, or I, | KVS (K-1-5) |
| X^{K31} represents S, N, or D. | LIS (K-1-6) |
| | EVS (K-1-7) |

Formula (K-1) is preferably one selected from the group consisting of formulas (K-1-1) to (K-1-7).

The light-chain CDR2 of the antibody or fragment thereof specifically bindable to the CTD of SARS-CoV-2 preferably contains the amino acid sequence selected from the group consisting of formulas (K-1-1) to (K-1-7), or an amino acid sequence having at least 90% identity to any of these amino acid sequences. The identity is preferably at least 91%, at least 92%, at least 93%, at least 94%, or at least 95%.

In an embodiment, the light-chain CDR3 of the antibody or fragment thereof specifically bindable to the CTD of SARS-CoV-2 preferably contains the following amino acid sequence:
the amino acid sequence represented by any of formulas (L-1) to (L-6) shown in Table 14,
an amino acid sequence having at least 90% (preferably at least 95%) identity to the amino acid sequence represented by any of formulas (L-1) to (L-6), or
an amino acid sequence formed of the amino acid sequence represented by any of formulas (L-1) to (L-6) with 1 to 3 (preferably 1 or 2, more preferably 1) amino acid mutations (preferably conservative substitution).

TABLE 14

| LSX^{L31}X^{L41}X^{L51}X^{L61}X^{L71}X^{L81}X^{L91}X^{L101}X^{L111} | LSX^{L32}X^{L42}SX^{L62}DAAX^{L102}V | LSRQSDDAASV |
|---|---|---|
| (L-1) wherein | (L-1-1) wherein | (L-1-2, SEQ ID NO: 134) |
| X^{L31} represents R, S, A, or G, | X^{L32} represents R or S, | LSSESDDAARV |
| X^{L41} represents Q, E, or D, | X^{L42} represents Q or E, | (L-1-3, SEQ ID NO: 135) |
| X^{L51} represents S or G, | X^{L61} represents D or N, | LSRESNDAAWV |
| X^{L61} represents D, S, or N, | X^{L102} represents S, R, or W. | (L-1-4, SEQ ID NO: 136) |
| X^{L71} represents D, G, or T, | LSADSSGTYRV (L-1-5, SEQ ID NO: 137) | |
| X^{L81} represents A, T, or Y, | LSRQSDTAASL (L-1-6, SEQ ID NO: 138) | |
| X^{L91} represents A, T, or Y, | LSRQGDTAASY (L-1-7, SEQ ID NO: 139) | |
| X^{L101} represents S, R, or W, | | |
| X^{L111} represents V, L, or Y. | | |
| LSX^{L33}X^{L43}X^{L53}X^{L63}X^{L73}X^{L83}X^{L93}X^{L103} | | LSGESDDAWV |
| (L-2) wherein | | (L-2-1, SEQ ID NO: 140) |
| X^{L33} represents R, S, A, or G, | | |
| X^{L43} represents Q, E, or D, | | |
| X^{L53} represents S or G, | | |
| X^{L63} represents D, S, or N, | | |
| X^{L73} represents D, G, or T, | | |
| X^{L83} represents A, T, or Y, | | |
| X^{L93} represents S, R, or W, | | |
| X^{L103} represents V, L, or Y. | | |
| X^{L14}QX^{L34}X^{L44}X^{L54}X^{L64}PX^{L84}X^{L94} | X^{L15}QX^{L35}THDPYT (L-3-1) | LQATHDPYT |
| (L-3) wherein | wherein | (L-3-2, SEQ ID NO: 141) |
| X^{L14} represents L or V, | X^{L15} represents L or V, | VQTTHDPYT |
| X^{L34} represents A or T, | X^{L35} represents A or T. | (L-3-3, SEQ ID NO: 142) |

| | |
|---|---|
| X^{L44} represents T or S, | LQATHNPVS (L-3-4, SEQ ID NO: 143) |
| X^{L54} represents H or N, | LQATHDPIT (L-3-5, SEQ ID NO: 144) |
| X^{L64} represents D, N, or V, | VQTSHVPLT (L-3-6, SEQ ID NO: 145) |
| X^{L84} represents Y, V, I, or L, | VQTTHVPLT (L-3-7, SEQ ID NO: 146) |
| X^{L94} represents T or S. | |

| | |
|---|---|
| QX^{L27}X^{L37}X^{L47}X^{L57}PX^{L77}X^{L87} | QTTNDPYT |
| (L-4) wherein | (L-4-1, SEQ ID NO: 147) |
| X^{L27} represents A or T, | |
| X^{L37} represents T or S, | |
| X^{L47} represents H or N, | |
| X^{L57} represents D, N, or V, | |
| X^{L77} represents Y, V, I, or L, | |
| X^{L87} represents T or S. | |

QVYDSSSFV (L-5, SEQ ID NO: 148)
WQGTDFPR (L-6, SEQ ID NO: 149)

In formula (L-1), X^{L31} is preferably R or S; X^{L41} is preferably Q or E; X^{L51} is preferably S; X^{L61} is preferably D or N; X^{L71} is preferably D; X^{L81} is preferably A; X^{L91} is preferably A; X^{L101} is preferably S, R, or W; and X^{L111} is preferably V.

Formula (L-1) is preferably formula (L-1-1) or one selected from the group consisting of formulas (L-1-2) to (L-1-7), and more preferably one selected from the group consisting of formula (L-1-2) to (L-1-4)

In formula (L-2), X^{L33} is preferably R, S, or G, more preferably G; X^{L43} is preferably Q or E, more preferably E; X^{L53} is preferably S; X^{L63} is preferably D; X^{L73} is preferably D; X^{L83} is preferably A; X^{L93} is preferably W; and X^{L103} is preferably V.

Formula (L-2) is preferably formula (L-2-1).

In formula (L-3), X^{L34} is preferably H; X^{L64} is preferably D or N, or D or V; and X^{L84} is preferably Y, V, or I, or Y or L.

Formula (L-3) is preferably formula (L-3-1) or one selected from the group consisting of formulas (L-3-2) to (L-3-7), and more preferably formula (L-3-2) or (L-3-3).

In formula (L-4), X^{L27} is preferably T; X^{L37} is preferably T; X^{L47} is preferably N; X^{L57} is preferably D; X^{L77} is preferably Y; and X^{L87} is preferably T.

Formula (L-4) is preferably formula (L-4-1).

The light-chain CDR3 of the antibody or fragment thereof specifically bindable to the CTD of SARS-CoV-2 preferably contains the amino acid sequence selected from the group consisting of formulas (L-1) to (L-3), (L-5), and (L-6), more preferably the amino acid sequence selected from the group consisting of formulas (L-1-1), (L-2-1), (L-3-1), (L-5), and (L 6), or the amino acid sequence selected from the group consisting of formulas (L-1-2) to (L-1-7), (L-2-1), (L-3-2), (L-3-3), (L-5), and (L-6), still more preferably the amino acid sequence selected from the group consisting of formulas (L-1-2) to (L-1-4), (L-2-1), (L-3-2), (L-3-3), (L-5), and (L-6), or an amino acid sequence having at least 90% identity to any of these amino acid sequences. The identity is preferably at least 91%, at least 92%, at least 93%, at least 94%, or at least 95%.

Table 15 shows preferable examples of combinations of the heavy-chain CDR1 to CDR3 and the light-chain CDR1 to CDR3 of the antibody or fragment thereof specifically bindable to the CTD of SARS-CoV-2.

TABLE 15

| | Heavy-chain | Heavy-chain | Heavy-chain | Light-chain | Light-chain | Light-chain |
|---|---|---|---|---|---|---|
| Combination | CDR1 | CDR2 | CDR3 | CDR1 | CDR2 | CDR3 |
| C-1 | (G-1) | (H-1) or | (I-1), | (J-1), | (K-1) | (L-1), |
| | | (H-2) | (I-2), | (J-2), or | | (L-2), |
| | | | (I-3), | (J-3) | | (L-3), |
| | | | (I-5), | | | (L-5), or |
| | | | (I-6), | | | (L-6) |
| | | | (I-7), | | | |
| | | | (I-8), or | | | |
| | | | (I-9) | | | |
| C-2 | (G-1-1) | (H-1-1) or | (I-1-1), | (J-1-1), | (K-1) | (L-1-1), |
| | | (H-2) | (I-2), | (J-2-1), or | | (L-2), |
| | | | (I-3), | (J-3) | | (L-3-1), |
| | | | (I-5), | | | (L-5), or |
| | | | (I-6), | | | (L-6) |
| | | | (I-7), | | | |
| | | | (I-8), or | | | |
| | | | (I-9) | | | |
| C-3 | (G-1-2), | (H-1-2), | (I-1-2) | (J-1-2), | (K-1-1), | (L-1-2), |
| | (G-1-3), | (H-1-3), | (I-1-3), | (J-1-3), | (K-1-2), | (L-1-3), |
| | (G-1-4), | (H-1-4), | (I-1-4), | (J-1-4), | (K-1-3), | (L-1-4), |
| | (G-1-5), | (H-1-5), | (I-1-5), | (J-2-2), | (K-1-4), | (L-2-1), |
| | (G-1-6), | (H-1-6), | (I-1-6), | (J-2-3), | (K-1-5), | (L-3-2), |
| | (G-1-7), | (H-1-7), | (I-2), | (J-2-4), | (K-1-6), or | (L-3-3), |
| | (G-1-8), or | (H-1-9), | (I-3), | (J-2-5), | (K-1-7) | (L-5), or |
| | (G-1-9) | (H-1-10), | (I-5), | (J-2-6), | | (L-6) |
| | | (H-1-11), | (I-6), | (J-2-7), or | | |
| | | (H-1-12), | (I-7), | (J-3) | | |
| | | (H-1-13), or | (I-8), or | | | |
| | | (H-2) | (I-9) | | | |
| C-4 | (G-1-2) | (H-1-2) | (I-1-2) | (J-1-2) | (K-1-2) | (L-1-2) |
| C-5 | (G-1-3) | (H-1-3) | (I-2) | (J-3) | (K-1-3) | (L-5) |
| C-6 | (G-1-4) | (H-1-4) | (I-1-3) | (J-1-3) | (K-1-4) | (L-1-3) |
| C-7 | (G-1-5) | (H-2) | (I-5) | (J-2-2) | (K-1-5) | (L-6) |
| C-8 | (G-1-6) | (H-1-6) | (I-9) | (J-2-3) | (K-1-6) | (L-3-2) |
| C-9 | (G-1-6) | (H-1-6) | (I-9) | (J-2-4) | (K-1-6) | (L-3-3) |
| C-10 | (G-1-8) | (H-1-9) | (I-6) | (J-2-5) | (K-1-7) | (L-3-3) |
| C-11 | (G-1-9) | (H-1-10) | (I-7) | (J-2-6) | (K-1-7) | (L-3-3) |
| C-12 | (G-1-7) | (H-1-7) | (I-3) | (J-1-3) | (K-1-4) | (L-2-1) |
| C-13 | (G-1-7) | (H-1-11) | (I-1-5) | (J-2-7) | (K-1-7) | (L-3-6) |
| C-14 | (G-1-4) | (H-1-5) | (I-8) | (J-1-4) | (K-1-2) | (L-1-5) |
| C-15 | (G-1-4) | (H-1-12) | (I-1-4) | (J-1-3) | (K-1-4) | (L-1-3) |
| C-16 | (G-1-4) | (H-1-13) | (I-1-6) | (J-1-3) | (K-1-1) | (L-1-3) |
| C-17 | (G-1-2), | (H-1-2), | (I-1-2), | (J-1-2), | (K-1-1), | (L-1-2), |
| | (G-1-3), | (H-1-3), | (I-1-3), | (J-1-3), | (K-1-2), | (L-1-3), |
| | (G-1-4), | (H-1-4), | (I-1-4), | (J-1-4), | (K-1-3), | (L-1-4), |
| | (G-1-5), | (H-1-5), | (I-1-5), | (J-1-5), | (K-1-4), | (L-1-5), |
| | (G-1-6), | (H-1-6), | (I-1-6), | (J-1-6), | (K-1-5), | (L-1-6), |
| | (G-1-7), | (H-1-7), | (I-1-7), | (J-1-7), | (K-1-6), or | (L-1-7), |
| | (G-1-8), | (H-1-8), | (I-1-8), | (J-2-2), | (K-1-7) | (L-2-1), |
| | (G-1-9), | (H-1-9), | (I-2), | (J-2-3), | | (L-3-2), |
| | (G-1-10), | (H-1-10), | (I-3), | (J-2-4), | | (L-3-3), |
| | (G-1-11), or | (H-1-11), | (I-4), | (J-2-5), | | (L-3-4), |
| | (G-2) | (H-1-12), | (I-5), | (J-2-6), | | (L-3-5), |
| | | (H-1-13), | (I-6), | (J-2-7), | | (L-3-6), |
| | | (H-1-14), | (I-7), | (J-2-8), | | (L-3-7), |
| | | (H-1-15), | (I-8), | (J-2-9), or | | (L-4-1), |
| | | (H-1-16), | (I-9), | (J-3) | | (L-5), or |
| | | (H-1-17), | (I-10), or | | | (L-6) |
| | | (H-2), or | (I-11) | | | |
| | | (H-3) | | | | |

It is also preferred that in combinations C-3 to C-8, one to three amino acids are mutated (preferably conservatively substituted) in the amino acid sequence of at least one CDR of the heavy-chain CDR1 to CDR3 and the light-chain CDR1 to CDR3.

The regions other than the heavy-chain CDR1 to CDR3 and the light-chain CDR1 to CDR3 are not particularly limited and can be of any amino acid sequence as long as the regions are usable in antibodies. For example, the constant region for use can be, but is not limited to, the constant region of IgG1, IgG2, IgG3, IgA1, IgA2, or IgM.

The antibody or fragment thereof specifically bindable to the NTD or CTD of SARS-CoV-2 has high bindability to the N protein of SARS-CoV-2; the bindability is, for example, 0.2 or higher, preferably 0.3 or higher, more preferably 0.4 or higher, and still more preferably 0.5 or higher, as indicated by absorbance measured according to the enzyme-linked immunosorbent assay (ELISA), described in the Examples below.

The antibody or fragment thereof specifically bindable to the NTD or CTD of SARS-CoV-2 has a binding affinity (Kd) for the N protein of SARS-CoV-2 of, for example, 50 nM or lower, preferably 10 nM or lower, and, for example, 1 pM or higher. The binding affinity Kd can be measured by using Biacore (trademark) T200 (Cytiva). Specifically, an amine coupling reaction using N′-(3-dimethylaminopropyl) carbodiimide hydrochloride (NHS) and N-hydroxysuccinimide (EDC) is used to immobilize an anti-guinea pig IgG antibody to carboxymethyl dextran on a CM5 sensor chip through a covalent bond. Then, an antibody or fragment thereof specifically bindable to the NTD or CTD of SARS-CoV-2 is injected into the sensor chip to bind the antibody or fragment thereof, and the N protein of SARS-CoV-2 is further injected into the sensor chip to allow the N protein to react with the antibody or fragment thereof specifically bindable to the NTD or CTD of SARS-CoV-2. The Kd is then calculated by analyzing the reaction signal.

### 4. Polynucleotide

The polynucleotide according to the present invention preferably contains the coding sequence of the antibody or fragment thereof specifically bindable to the NTD or CTD of SARS-CoV-2. An example of the coding sequence is, but is not limited to, the base sequence encoding the antibody or fragment thereof containing the heavy-chain CDR1 to CDR3 and the light-chain CDR1 to CDR3 described in section 3 above.

In an embodiment, the polynucleotide according to the present invention preferably contains an expression cassette of the antibody or fragment thereof specifically bindable to the NTD or CTD of SARS-CoV-2. The expression cassette is not particularly limited to a specific one as long as the expression cassette allows for expression in a host cell, and contains, for example, a promoter and coding sequences placed under control of the promoter.

The promoter is not limited and can be suitably selected according to the type of the host cell. For example, various pol II promoters are usable. Examples of pol II promoters include, but are not limited to, the CMV promoter, the EF1 promoter, the SV40 promoter, and the MSCV promoter. Examples of promoters also include other promoters, such as tryptophan promoters (e.g., trc and tac), the lac promoter, the 17 promoter, the 15 promoter, the 13 promoter, the SP6 promoter, the arabinose-induced promoter, the cold-shock promoter, and the tetracycline-induced promoter.

The expression cassette may contain other elements as needed. Examples of other elements include a multiple cloning site (MCS), a drug-resistant gene, a replication origin, an enhancer sequence, a repressor sequence, an insulator sequence, a reporter protein coding sequence, and a drug-resistant gene coding sequence. These may be one type alone or a combination of two or more.

For example, the polynucleotide according to the present invention can be in the form of a vector. An appropriate vector is selected according to the intended use, host cell type, etc. For example, E. coli vectors include M13 phage or variants thereof, λ phage or variants thereof, and pBR322 or variants thereof (e.g. pB325, pAT153, pUC8); yeast vectors include pYepSecl, pMFa, pYES2, and pPIC3.5K; insect vectors include pAc, and pVL; and mammalian vectors include pcDNA, pCDM8, and pMT2PC.

### 5. Cell

The cell according to the present invention preferably contains the polynucleotide described in section 4 above. The cell includes, for example, Escherichia coli such as Escherichia coli K12, bacteria of the genus Bacillus, such as Bacillus subtilis MI114, yeast, such as Saccharomyces cerevisiae AH22, Sf cell lines derived from Spodoptera frugiperda, High Five cell lines derived from Trichoplusia ni, insect cells, such as olfactory nerve cells, and animal cells. Animal cells are preferably cultured cells of mammalian origin, and specifically include COST cells, CHO cells, HEK293 cells, Expi293 cells, 293F cells, 293T cells, 293FT cells, Hela cells, PC12 cells, N1E-115 cells, and SH-SY5Y cells.

In an embodiment, the cell according to the present invention preferably expresses the antibody or fragment thereof specifically bindable to the NTD or CTD of SARS-CoV-2.

In an embodiment, the cell according to the present invention preferably secretes the antibody or fragment thereof specifically bindable to the NTD or CTD of SARS-CoV-2 or preferably has the antibody or fragment thereof specifically bindable to the NTD or CTD of SARS-CoV-2 on the cell surface.

### 6. Reagent or Drug

The reagent or drug according to the present invention preferably contains an antibody or fragment thereof specifically bindable to the NTD or CTD of SARS-CoV-2, a polynucleotide containing the coding sequence of the antibody or fragment thereof, or a cell containing the polynucleotide. The reagent or drug according to the present invention preferably further contains a pharmaceutically acceptable excipient or carrier and/or additives.

Examples of excipients or carriers include starch, lactose, crystalline cellulose, sorbitol, calcium hydrogen phosphate, water, ethanol, (poly)ethylene glycol, (poly)propylene glycol, glycerol, and vegetable oil. These can be used singly or in combination of two or more.

Examples of additives include buffers, tonicity agents, thickeners, chelating agents, emulsifiers, colorants, and preservatives. These can be used singly or in a combination of two or more.

The reagent or drug according to the present invention may be an antibody or fragment thereof specifically bindable to the NTD or CTD of SARS-CoV-2 that is dissolved or dispersed in a solvent or lyophilized.

### 7. Kit for Detecting the N Protein of SARS-CoV-2

The kit for detecting the N protein of SARS-CoV-2 according to the invention preferably contains the antibody or fragment thereof specifically bindable to the NTD or CTD of SARS-CoV-2. The antibody or fragment thereof may be in the form of a reagent, such as described in section 6 above.

The kit preferably contains the following:
two or more antibodies or fragments thereof specifically bindable to the NTD of SARS-CoV-2, and the two or more antibodies or fragments thereof each recognize a different epitope of the NTD, and/or
two or more antibodies or fragments thereof specifically bindable to the CTD of SARS-CoV-2, and the two or more antibodies or fragments thereof each recognize a different epitope of the CTD.

The two or more antibodies or fragments thereof specifically bindable to the NTD preferably differ from each other in terms of the amino acid sequence of at least one CDR of the heavy-chain CDR1 to CDR3 and the light-chain CDR1 to CDR3. In an embodiment, a portion of the two or more antibodies or fragments thereof specifically bindable to the NTD (“the primary anti-NTD antibody or fragment thereof” below) is preferably immobilized, and the remaining portion (“the secondary anti-NTD antibody or fragment thereof” below) is preferably labeled. Examples of the solid phase on which the primary anti-NTD antibody or fragment thereof is immobilized include, but are not limited to, beads, wells, chips, and strips. The method for immobilizing the primary anti-NTD antibody or fragment thereof is not particularly limited. An example is a method of bringing a biotinylated primary anti-NTD antibody or fragment thereof into contact with the surface of a solid phase containing avidin. The labeling substance for labeling the secondary anti-NTD antibody or fragment thereof is not particularly limited, and includes, for example, enzymes such as peroxidase (HRP), and alkaline phosphatase (ALP)

The primary and secondary anti-NTD antibodies or fragments thereof can be selected, for example, from the antibodies or fragments thereof listed in section 2 above, preferably from N-1 in Table 8, more preferably from N-2 in Table 8, still more preferably from N-3 in Table 8, and particularly preferably from combinations of N-4 and N-5, combinations of N-4 and N-6, combinations of N-4 and N-7, combinations of N-5 and N 6, combinations of N-5 and N-7, combinations of N-6 and N-7, combinations of N-6 and N-13, combinations of N-10 and N-12, combinations of N-5 and N-8, and combinations of N-9 and N-10 combinations in Table 8 (wherein the amino acid sequences of the heavy-chain CDR1 to CDR3 and the light-chain CDR1 to CDR3 of N-4 to N-10, N-12, and N-13 may be amino acid sequences having at least 90% identity to these amino acid sequences, or amino acid sequences formed of these amino acid sequences with 1 to 3 amino acid mutations).

The two or more antibodies or fragments thereof specifically bindable to the CTD preferably differ from each other in terms of the amino acid sequence of at least one CDR of the heavy-chain CDR1 to CDR3 and the light-chain CDR1 to CDR3. In an embodiment, a portion of the two or more antibodies or fragments thereof specifically bindable to the CTD (“the primary anti-CTD antibody or fragment thereof” below) is preferably immobilized, and the remaining portion (“the secondary anti-CTD antibody or fragment thereof” below) is preferably labeled. Examples of the solid phase on which the primary anti-CTD antibody or fragment thereof is immobilized include, but are not limited to, beads, wells, chips, and strips. The method for immobilizing the primary anti-CTD antibody or fragment thereof is not particularly limited. An example is a method of bringing a biotinylated primary anti-CTD antibody or fragment thereof into contact with the surface of a solid phase containing avidin. The labeling substance for labeling the secondary anti-CTD antibody or fragment thereof is not limited, and includes, for example, enzymes such as peroxidase (HRP), and alkaline phosphatase (ALP).

The primary and secondary anti-CTD antibodies or fragments thereof can be selected, for example, from the antibodies or fragments thereof listed in section 3 above, preferably from C-1 in Table 15, more preferably from C-2 in Table 15, still more preferably from C-3 in Table 15, and particularly preferably from combinations of C-4 and C-5, combinations of C-4 and C-6, combinations of C-4 and C-7, combinations of C-5 and C-6, combinations of C-5 and C-7, combinations of C-6 and C-7, combinations of C-4 and C-12, combinations of C-9 and C-11, and combinations of C-7 and C-16 in Table 15 (wherein the amino acid sequences of the heavy-chain CDR1 to CDR3 and the light-chain CDR1 to CDR3 of C-4 to C-7, C-9, C-11, C-12, and C-16 may be amino acid sequences having at least 90% identity to these amino acid sequences, or amino acid sequences formed of these amino acid sequences with 1 to 3 amino acid mutations).

The kit is preferably a kit for detecting the N protein of SARS-CoV-2 in a test sample. The test sample may be a biological or non-biological sample. The test sample includes not only the samples as collected but also those samples that have undergone pretreatment, such as removal of foreign substances.

Examples of biological samples include, but are not limited to, blood, serum, plasma, bone marrow fluid, lymph fluid, tears, nasal discharge, nasal lavage fluid, nasal swab fluid, saliva, gargle fluid, sputum, throat swab fluid, sweat, tracheal aspirate, bronchus lavage fluid, pleural effusion, ascites fluid, amniotic fluid, intestinal lavage fluid, urine, feces, cell extracts, tissue extracts, and organ extracts.

Examples of non-biological samples include, but are not limited to, samples taken from environmental surfaces such as faucets, handles, handrails, suspension straps, switches, walls, floors, desks, chairs, and toilet seats.

Examples of the method for detecting the N protein of SARS-CoV-2 by using the kit include, but are not limited to, immunochromatography, enzyme immunoassay, chemiluminescent immunoassay, chemiluminescent enzyme immunoassay, radioimmunoassay, electrochemiluminescent immunoassay, immunonephelometry, and latex agglutination.

The kit may further contain other substances (e.g., buffers, enzyme solutions, solutions for dilution, and secondary antibodies), instruments for use, an instruction manual, etc., for example, according to the preparation method for the test sample, or the detection method for the N protein of SARS-CoV-2.

### 8. Method for Detecting the N Protein of SARS-CoV-2

The method for detecting the N protein of SARS-CoV-2 preferably includes the following steps (1) to (3):
(1) immobilizing the primary antibody or fragment thereof specifically bindable to the NTD or CTD onto a solid phase,
(2) applying the secondary antibody or fragment thereof that is specifically bindable to the N protein of SARS-CoV-2, and that is specifically bindable to the amino acid sequence of SEQ ID NO: 1 or 2 to the solid phase (wherein the secondary antibody or fragment thereof recognizes a different epitope in the amino acid sequence to which the primary antibody or fragment thereof is specifically bindable, and the secondary antibody or fragment thereof is labeled with a labeling substance), and
(3) detecting a label derived from the labeling substance of the secondary antibody or fragment thereof bound to the solid phase via the nucleocapsid protein.

The primary and secondary antibodies or fragments thereof are preferably the primary and secondary anti-NTD antibodies or fragments thereof and/or the primary and secondary anti-CTD antibodies or fragments thereof described in section 7 above.

The solid phase and the labeling substance can be selected, for example, from those described in section 7 above.

### Examples

### Obtainment of Antibody-Producing Cells by Animal Immunization

195 μg of SARS-CoV-2 N protein suspended in PBS was mixed with an adjuvant, TiterMAX Gold (Funakoshi Co., Ltd.), and the mixture was administered intramuscularly into the left and right hind limbs of guinea pigs as an initial immunization. Fourteen days after the initial immunization, 100 pg of SARS-CoV-2 N protein suspended in PBS was mixed with TiterMAX Gold, booster immunization was intramuscularly performed in the right and left hind limbs of guinea pigs. Thirty-one days after the booster immunization, booster immunization was performed again by intramuscularly administering 100 pg of SARS-CoV-2 N protein suspended in PBS to the right and left hind limbs of guinea pigs. Eight days after the second booster immunization, two guinea pigs were subjected to total blood sampling and euthanasia under anesthesia. Thereafter, lumbar lymph nodes were removed.

### Sorting of Plasma Cells Producing Anti-SARS-Cov2 N Protein Antibody

Sorting was performed according to the method of US patent application publication No. 2013-0029325, i.e., Fluorescent Probe for Plasma Cell Identification and Isolation, and Plasma Cell Identification or Isolation Method Using the Probe (ERIAA method).

Cells obtained from extracted lumbar lymph nodes were immobilized in a formaldehyde PBS solution on ice for 10 minutes. Immobilized cells were stained with Dylight488-labeled SARS-CoV-2 N protein, Dylight 550-labeled SARS-CoV N protein, Dylight 650-labeled anti-guinea pig IgG antibody, and DAPI (4′,6-diamidino-2-phenylindole); and plasma cells showing strong positivity for SARS-CoV-2 N protein, showing negativity for SARS-CoV N protein, and showing strong positivity for anti-guinea pig IgG were sorted by flow cytometry.

### Synthesis of Antibody Variable Region Gene Fragment

For cells obtained by sorting, mRNA extraction, cDNA synthesis, and amplification of antibody variable region gene fragments were performed according to the method of Kurosawa et al. (MAGrand method) (Rapid production of antigen-specific monoclonal antibodies from a variety of animals, BMC Biology 2012, 10:80).

### Production of Antibody Gene Expression Unit

From the amplified antibody variable region gene fragments, antibody gene expression units were produced according to the method of US patent application publication No. 2013-0023009, i.e., Specific Method for Preparing Joined DNA Fragments Including Sequences Derived from Target Genes (TS-jPCR method).

### Transient Expression of Antibody in 293FT Cells

After the prepared antibody gene expression unit, FuGene HD Transfection Reagent (trademark), and Opti-MEM (trademark) were mixed at a liquid volume ratio of 1:1:50 and allowed to stand at ambient temperature for 20 minutes, the mixture was added to 293FT cells suspended in 10% (w/v) FBS/IMDM medium to introduce the antibody gene expression unit. The 293FT cells in which the antibody gene expression unit had been introduced were seeded in a 24-well plate and incubated at 37° C. in an 8% (v/v) CO₂ environment for 2 days, after which a culture supernatant was collected. Antibodies released in the culture supernatant were used for subsequent screening.

### Screening of Antibody by ELISA

Screening was performed by evaluating the reaction intensity of each antibody with SARS-CoV-2 N protein by ELISA. In ELISA, immobilization was performed on the bottom of a 96-well plate so that SARS-CoV-2 N protein was at 15 ng/well, and 100 μL of the culture supernatant of 293FT cells in which the antibody was transiently expressed was added to perform an antigen-antibody reaction for 2 hours at room temperature. Guinea pig antibodies bonded to antigens were detected using a goat anti-guinea pig antibody conjugated with horseradish peroxidase, and absorbance was measured at 450 nm using a microplate reader.

### Reactivity Evaluation Screening of Antibody by Immunostaining

To confirm whether the obtained antibodies recognize the N-terminal domain (NTD) or the C-terminal domain (CTD) of SARS-CoV-2 N protein, a DNA fragment encoding NTD and a DNA fragment encoding CTD were transiently expressed in 293FT cells. By adding obtained antibodies to perform immunostaining, the reactivity of antibodies with the DNA fragment encoding NTD and with the DNA fragment encoding CTD was evaluated.

Table 16 shows the amino acid sequences of the NTD and CTD.

TABLE 16

| | |
|---|---|
| N- | MSDNGPQNQRNAPRITFGGPSDSTGSNQNGERS |
| Terminal | GARSKQRRPQGLPNNTASWFTALTQHGKEDLKF |
| Domain | PRGQGVPINTNSSPDDQIGYYRRATRRIRGGDG |
| (NTD) | KMKDLSPRWYFYYLGTGPEAGLPYGANKDGIIW |
| | VATEGALNTPKDHIGTRNPANNAAIVLQLPQGT |
| | TLPKGFY (SEQ ID: No. 1) |
| C- | TKKSAAEASKKPRQKRTATKAYNVTQAFGRRGP |
| Terminal | EQTQGNFGDQELIRQGTDYKHWPQIAQFAPSAS |
| Domain | AFFGMSRIGMEVTPSGTWLTYTAAIKLDDKDPN |
| (CTD) | FKDQVILLNKHIDAYKTEPPTEPKKDKKKKADE |
| | TQALPQRQKKQQTVTLLPAADLDDFSKQLQQSM |
| | SSADSTQA (SEQ ID: No. 2) |

Using a plasmid containing an ORF domain of SARS-CoV-2 N protein as a template, a DNA fragment encoding NTD and a DNA fragment encoding CTD were produced by PCR. The prepared DNA fragment (50 ng/μL), FuGene HD Transfection Reagent (trademark) (produced by Promega Co., Ltd.), and Opti-MEM (trademark) (Thermo Fisher Scientific Inc.) were mixed at a liquid volume ratio of 1:1:50. The mixture was allowed to stand at ambient temperature for 20 minutes and added to 293FT cells that had been suspended in 10% (w/v) FBS/IMDM medium.

The 293FT cells to which the DNA fragments had been introduced were seeded in a collagen-coated 96-well plate, followed by incubation at 37° C.; under an 8% (v/v) CO₂ environment for 2 days. Thereafter, the medium was removed, followed by washing with PBS. A 3% (w/v) formaldehyde PBS solution was added in an amount of 100 μL/well and allowed to stand at ambient temperature for 20 minutes to immobilize the cells. Further, a 0.1% (w/v) Triton PBS solution was added in an amount of 200 μL/well and allowed to stand at room temperature for 5 minutes to perform cell membrane permeabilization. After the solution in the wells was removed, 100 μL of culture supernatant of 293FT cells in which the antibody was transiently expressed were added, and allowed to stand at room temperature for 3 hours for reaction. After the culture supernatant was removed, and washing with PBS was performed, a Dylight594-labeled anti-guinea pig IgG antibody was added, and the mixture was allowed to stand at room temperature for 1 hour under shielded light for reaction. After further washing with PBS, the nuclei were stained with PBS containing DAPI.

By observing the reactivity of immobilized SARS-CoV-2 N protein (full length) and the antibody, and the reactivity of the antibody and cells expressing DNA fragments each encoding NTD or CTD using the method described above, it was determined whether each antibody had NTD reactivity or CTD reactivity.

### Transient Expression of Antibody in Expi293 Cells and Purification

For antibodies having high reactivity as a result of evaluation by ELISA, antibodies were produced by transfection of the produced gene expression unit into Expi293 cells. Expi293 Expression Medium (GIBCO) and Expi Fectamine 293 Transfection Kit (GIBCO) were used for the transfection. After 50 pg of the produced gene expression unit of each antibody, 3 mL of OptiMEM I, and 0.16 mL of Expi Fectamine 293 were mixed per E250 culture flask, and allowed to stand for 10 min, the resultant was added to Expi293 cells, thus performing gene introduction. After culture for one day, Expi Fectamine 293 Transfection Enhancer was feed-added, followed by culturing for 4 days. Thereafter, the supernatant was collected and purified in a protein A column to obtain antibodies.

Of antibodies obtained by screening 260 clones of the obtained antibodies, Tables 17 and 18 show the amino acid sequences (based on the IMGT method) of heavy-chain CDR 1 to 3 and light-chain CDR 1 to 3 of 16 clones (N1 to N16) of the antibodies specifically conjugated to NTD and 27 clones (C1 to C27) of the antibodies specifically conjugated to CTD.

TABLE 17

| Clone | Heavy chain (H chain) | | | Light chain (L chain) | | |
|---|---|---|---|---|---|---|
| No. | CDR1 | CDR2 | CDR3 | CDR1 | CDR2 | CDR3 |
| N1 | GFTFDDYG | ISYSGGRT | ARDGGSYYSPINFQF | SEHRSYY | LKSDGSH | GVSYSGGHNI |
| | (A-1-2) | (B-1-2) | (C-1-2) | (D-1-2) | (E-1-2) | (F-1-2) |
| N2 | GFTFSDFW | INYDGDRT | ATNYNFQY | SDYSHYS | VGTSDIVG | GADYSGASSYV |
| | (A-1-3) | (B-1-3) | (C-2-2) | (D-1-3) | (E-2) | (F-2-1) |
| N3 | GFTFNNYW | INGDSTKI | ATSGPMDV | SQGINKW | KAN | QYDSTPPLT |
| | (A-1-4) | (B-1-4) | (C-2-3) | (D-1-4) | (E-3-1) | (F-5) |
| N4 | GFTFDDFG | IDYSGGTT | SRDGGSYHSPISCQY | SGHSSYY | VKSDGSH | GVSFSGGHGV |
| | (A-1-5) | (B-1-5) | (C-1-3) | (D-1-5) | (E-1-3) | (F-1-3) |
| N5 | GFSITTNGYT | IWYDADT | ARSIIAEIPTIFTFP | NIGGKT | SHN | QVWDSYTYV |
| | (A-2-2) | (B-2-2) | PFDV | (D-3) | (E-3-2) | (F-6) |
| | | | (C-3-1) | | | |
| N6 | GFSLTSYS | IWSGGRI | ARLWSGYALDI | SEYKHYN | VKSDGSL | GANHNIGESYGYV |
| | (A-1-8) | (B-2-3) | (C-4) | (D-1-1) | (E-1-5) | (F-3-1) |
| N7 | GFTFDDYG | ISYSGGST | ARDGGSYSSPINFQY | SEHSSYY | LKSDGSH | GVSYSGGHNV |
| | (A-1-2) | (B-1-6) | (C-1-4) | (D-1-6) | (E-1-2) | (F-1-4) |
| N8 | GFTFDDYG | ISPSGGST | ARDGGSVTSPIYFQV | SEHSSYY | LNSYGSH | GVSVSGGHNL |
| | (A-1-2) | (B-1-10) | (C-1-6) | (D-1-6) | (E-1-6) | (F-1-5) |
| N9 | GFTFDDYG | ISYSGGNT | ARDGGSVKSPIYFQY | SEHSSYY | LNSKGSH | GVSLKGGHNL |
| | (A-1-2) | (B-1-11) | (C-1-7) | (D-1-6) | (E-1-7) | (F-1-6) |
| N10 | GFTFSDYT | IWYDGTKM | AKYYDYDYTGFDV | SQSLFYS | WAS | QFINGPLT |
| | (A-1-9) | (B-1-12) | (C-5-1) | GKNKDL | (E-3-3) | (F-7) |
| | | | | (D-2-1) | | |
| N11 | GFTFSDYW | IHPDGGST | VSPSGAGLDV | SEHSSYY | LKSDGSH | GVGYNNKYV |
| | (A-1-7) | (B-1-8) | (C-7-1) | (D-1-6) | (E-1-2) | (F-4-2) |
| N12 | GFTFSDYW | ISPDGGGT | SPSAVGFDV | SDHSSYY | LKSDGSH | GVGYSGGYV |
| | (A-1-7) | (B-1-9) | (C-8-1) | (D-1-7) | (E-1-2) | (F-4-3) |
| N13 | GFTFSNYY | ISGDSNYI | VSPLLVVHGPFFQY | SEHSSYY | IESDGSH | GVSYSGGYV |
| | (A-1-6) | (B-1-7) | (C-6-1) | (D-1-6) | (E-1-4) | (F-4-4) |
| N14 | GFTFSNYY | ISGDTNYI | VSPLLVYHGPFFQY | SDHSSYY | IESDGSH | GVKYSGGVV |
| | (A-1-6) | (B-1-13) | (C-6-2) | (D-1-7) | (E-1-4) | (F-4-5) |
| N15 | GFTFSNYY | ISGDTNYI | VKPLLVLHGPFFQS | SDHSSYY | IESKGSH | GVSYSGGYY |
| | (A-1-6) | (B-1-13) | (C-6-3) | (D-1-7) | (E-1-8) | (F-4-6) |
| N16 | GFTFDDYG | ISYSGGST | SRDGGSYSSPINFQY | SEHSSYY | VKSDGSH | GVSYSGGHNV |
| | (A-1-2) | (B-1-6) | (C-1-5) | (D-1-6) | (E-1-3) | (F-1-4) |

TABLE 18

| Clone | Heavy chain (H chain) | | | Light chain (L chain) | | |
|---|---|---|---|---|---|---|
| No. | CDR1 | CDR2 | CDR3 | CDR1 | CDR2 | CDR3 |
| C1 | GFIFSNYF | ISGDSTYI | TINWGDV | KLSNQY | KDS | LSRQSDDAASV |
| | (G-1-2) | (H-1-2) | (I-1-2) | (J-1-2) | (K-1-2) | (L-1-2) |
| C2 | GFTFNTYT | IGNSGSKT | ARTSRIVADL | NIGGKA | NGN | QVYDSSSFV |
| | (G-1-3) | (H-1-3) | VTMGYALDF | (J-3) | (K-1-3) | (L-5) |
| | | | (I-2) | | | |
| C3 | GFTFSNYY | ISGDGSNI | STLGVGV | KLSERY | NDS | LSSESDDAARV |
| | (G-1-4) | (H-1-4) | (I-1-3) | (J-1-3) | (K-1-4) | (L-1-3) |
| C4 | GLSLTNYT | MWSDGDT | ATSEY | SESLLHSDGNTY | KVS | WQGTDFPR |
| | (G-1-5) | (H-2) | (I-5) | (J-2-2) | (K-1-5) | (L-6) |
| C5 | GFFFSNSW | IKGDSSIT | GSGWV | SQSLLHGDGKTY | LIS | LQATHDPYT |
| | (G-1-10) | (H-1-8) | (I-9) | (J-2-8) | (K-1-6) | (L-3-2) |
| C6 | GFSFSNSW | IKGDSSII | GSGWV | SQSLLHSDGKTY | LIS | LQATHDPYT |
| | (G-1-6) | (H-1-6) | (I-9) | (J-2-3) | (K-1-6) | (L-3-2) |
| C7 | GFSFSNSW | IKGDSSII | GSGWY | SQSLLHSDGNTY | LIS | LQATHNPVS |
| | (G-1-6) | (H-1-6) | (I-10) | (J-2-9) | (K-1-6) | (L-3-4) |
| C8 | GFSFSNSW | IKGDSSII | GSGWY | SQSLVHSDGKTY | LIS | LQATHDPIT |
| | (G-1-6) | (H-1-6) | (I-10) | (J-2-4) | (K-1-6) | (L-3-5) |
| C9 | GFSFSNSW | IKGDSSII | GSGWV | SQSLLHSDGKTY | LIS | LQATHDPYT |
| | (G-1-6) | (H-1-6) | (I-9) | (J-2-4) | (K-1-6) | (L-3-3) |
| C10 | GFSFSNSW | IKGDSSTI | GSGWV | SQSLLHSDGKTY | LIS | LQATHDPYT |
| | (G-1-6) | (H-1-14) | (I-9) | (J-2-4) | (K-1-6) | (L-3-3) |
| C11 | GFSFSNSW | IKGDSSII | GSGWV | SQSLLHSDGKTY | LIS | LQATHDPYT |
| | (G-1-6) | (H-1-6) | (I-9) | (J-2-4) | (K-1-6) | (L-3-3) |
| C12 | GFTFDDYG | IGYSGASI | AREGVITVGT | ALPKKY | KDS | LSADSSGTYRV |
| | (G-1-11) | (H-1-16) | WGDV | (J-1-5) | (K-1-2) | (L-1-5) |
| | | | (I-4) | | | |
| C13 | GFTFRNHW | INSDGSNT | TTATDV | SQSLLDSDGYTY | EVS | VQTTHDPYT |
| | (G-1-8) | (H-1-9) | (I-6) | (J-2-5) | (K-1-7) | (L-3-3) |
| C14 | GFTFSNHW | INTDGSST | TPATDV | SQSLLYSDGYTY | EVS | VQTTHDPYT |
| | (G-1-9) | (H-1-10) | (I-7) | (J-2-6) | (K-1-7) | (L-3-3) |
| C15 | GFTFSNHW | INSDGSST | TTATDV | SQSLLYSDGYTY | EVS | QTTNDPYT |
| | (G-1-9) | (H-1-15) | (I-6) | (J-2-7) | (K-1-7) | (L-4-1) |
| C16 | GFTFSNYW | INPDDGSP | ARDVVKTGTT | KLSERY | NDS | LSGESDDAWV |
| | (G-1-7) | (H-1-7) | GLPFDL | (J-1-3) | (K-1-4) | (L-2-1) |
| | | | (I-3) | | | |
| C17 | GFTFSNYW | INPDDGSP | ARDVVKTGTT | KLSERY | NDS | LSGESDDAWV |
| | (G-1-7) | (H-1-7) | GLPFDL | (J-1-3) | (K-1-4) | (L-2-1) |
| | | | (I-3) | | | |
| C18 | GFTFSNYW | IKGDSRTI | STTGFDV | SQSLLYSDGYTF | EVS | VQTSHVPLT |
| | (G-1-7) | (H-1-11) | (I-1-5) | (J-2-7) | (K-1-7) | (L-3-6) |
| C19 | GFTFSNYW | IKGDSSTI | STTGFDV | SQSLLYSDGYTF | EVS | VQTTHVPLT |
| | (G-1-7) | (H-1-14) | (I-1-5) | (J-2-7) | (K-1-7) | (L-3-7) |
| C20 | GFTFSNYY | ISGDSSNI | SPMTIHGLDT | KLSKEY | KDS | LSRESNDAAWV |
| | (G-1-4) | (H-1-5) | (I-8) | (J-1-4) | (K-1-2) | (L-1-4) |
| C21 | GFTFSNYY | ISGTSSNI | TTLGVGV | KLSERY | NDS | LSSESDDAARV |
| | (G-1-4) | (H-1-17) | (I-1-4) | (J-1-3) | (K-1-4) | (L-1-3) |
| C22 | GFTFSNYY | ISGTSSNI | TTLGVGV | KLSERY | NDS | LSSESDDAARV |
| | (G-1-4) | (H-1-17) | (I-1-4) | (J-1-3) | (K-1-4) | (L-1-3) |
| C23 | GFTFSNYY | ISGASSNI | TTLGVGV | KLSERY | NDS | LSSESDDAARV |
| | (G-1-4) | (H-1-12) | (I-1-4) | (J-1-3) | (K-1-4) | (L-1-3) |
| C24 | GFTFSNYY | ISGDSTYI | TIDWGNV | KLSTQY | KDS | LSRQSDTAASL |
| | (G-1-4) | (H-1-2) | (I-1-8) | (J-1-6) | (K-1-2) | (L-1-6) |
| C25 | GFIFSNYF | ISGDGSNI | TITWGDV | KLSDQY | KDS | LSRQGDTAASY |
| | (G-1-2) | (H-1-4) | (I-1-7) | (J-1-7) | (K-1-2) | (L-1-7) |
| C26 | KLSERY | NDS | LSSESDDAAR | KLSERY | NDS | LSSESDDAARV |
| | (G-2) | (H-3) | V | (J-1-3) | (K-1-4) | (L-1-3) |
| | | | (I-11) | | | |
| C27 | GFTFSNYY | ISGDSSNV | STLGNFL | KLSERY | NDD | LSSESDDAARV |
| | (G-1-4) | (H-1-13) | (I-1-6) | (J-1-3) | (K-1-1) | (L-1-3) |

The effects of the present invention are explained below using some clones having a reactivity with NTD or CTD; however, the present invention is not limited to these clones. Table 19 shows the results of immunostaining and ELISA for typical clones. The use of other clones can also attain similar activity effects.

TABLE 19

| | Immunostaining | | ELISA |
|---|---|---|---|
| Clone No. | NTD | CTD | Absorbance value |
| N1 | + | − | 0.477 |
| N2 | + | − | 0.184 |
| N3 | + | − | 0.121 |
| N16 | + | − | 0.212 |
| N13 | + | − | 0.860 |
| N11 | + | − | 0.921 |
| N6 | + | − | 0.429 |
| N7 | + | − | 0.672 |
| N12 | + | − | 0.959 |
| N4 | + | − | 0.796 |
| C1 | − | + | 1.985 |
| C2 | − | + | 1.402 |
| C3 | − | + | 0.688 |
| C4 | − | + | 0.737 |
| C9 | − | + | 0.443 |
| C27 | − | + | 1.249 |
| C16 | − | + | 0.331 |
| C14 | − | + | 1.301 |
| C20 | − | + | 1.686 |
| C6 | − | + | 0.443 |
| C18 | − | + | 1.338 |
| C13 | − | + | 1.040 |
| C23 | − | + | 0.777 |

Sandwich ELISA for NP Antigen with Progressive Degradation

Each biotin-labelled clone (immobilized antibody) was diluted with TBS to 5 pg/mL, and added to a 96-well microplate in an amount of 50 μL/well, followed by coating for 2 hours at room temperature. After blocking with 1% (w/v) BSA-TBS-T and subsequent washing with TBS-T, each 0.25 pg/mL alkaline phosphatase-labeled clone (detection antibody) and a 10 ng/mL antigen solution (1% (w/v) TBS-T dilution) were each added in an amount of 50 μL/well to perform reaction at room temperature for 2 hours. As an antigen solution, in addition to Sample 1 of the full-length SARS-CoV-2 N protein, Sample 2, in which the total amount of the SARS-CoV-2 N protein was replaced with the equivalent mol amount of NTD and the equivalent mol amount of CTD, as a mimic of the sample in which the SARS-CoV-2 N protein was degraded, was used. After washing with TBS-T, pNPP was added in an amount of 100 μL/well as a chromogenic substrate, and the reaction was performed for 15 minutes at room temperature, after which the absorbance at 405 nm was measured using a microplate reader. Table 20 shows the results. The percentage (%) in Table 20 represents the relative absorbance value to the absorbance measurement value of Sample 1.

TABLE 20

| Measure- | | | Sample 1 | Sample 2 |
|---|---|---|---|---|
| ment | Immobilized | Detection | (0% of antigen | (100% of antigen |
| No. | antibody | antibody | is degraded.) | is degraded.) |
| 1 | N1 | N2 | 100% | 38% |
| 2 | N3 | N16 | 100% | 22% |
| 3 | N13 | N11 | 100% | 77% |
| 4 | N2 | N6 | 100% | 17% |
| 5 | N11 | N7 | 100% | 19% |
| 6 | C1 | C2 | 100% | 45% |
| 7 | C3 | C4 | 100% | 32% |
| 8 | C1 | C16 | 100% | 90% |
| 9 | C9 | C14 | 100% | 25% |
| 10 | C27 | C4 | 100% | 47% |
| 11 | N1 | C2 | 100% | 6% |
| 12 | N1 | C4 | 100% | 5% |
| 13 | N3 | C2 | 100% | 1% |
| 14 | N3 | C4 | 100% | 5% |
| 15 | N13 | C16 | 100% | 0% |
| 16 | N2 | C14 | 100% | 0% |
| 17 | N11 | C4 | 100% | 0% |
| 18 | C1 | N2 | 100% | 3% |
| 19 | C1 | N16 | 100% | 3% |
| 20 | C3 | N2 | 100% | 2% |
| 21 | C3 | N16 | 100% | 3% |
| 22 | C1 | N11 | 100% | 1% |
| 23 | C9 | N6 | 100% | 5% |
| 24 | C27 | N7 | 100% | 0% |

When two types of antibodies recognizing NTD were combined, and when two types of antibodies recognizing CTD were combined, it was assumed that the antigen fragment had a conformational change in Sample 2, which was a mimic of the sample in which the SARS-CoV-2 N protein was degraded, and that the reactivity of the antigen fragment was reduced, and a reduction in the absorbance measurement value was observed; however, the degree of reduction was low. In contrast, when the NTD recognition antibody was combined with the CTD recognition antibody, a significant reduction in the absorbance measurement value was confirmed in Samples 2 to 4.

### Sandwich ELISA Using Oligoclonal Antibody

An immobilized antibody mixed with multiple biotin-labeled clones was diluted to 5 pg/mL with TBS, and added to a 96-well microplate in an amount of 50 μL/well, followed by coating for 2 hours at room temperature. After blocking with 1% (w/v) BSA-TBS-T and subsequent washing with TBS-T, a 0.25 pg/mL detection antibody mixed with multiple clones labeled with alkaline phosphatase, and a 10 ng/mL antigen solution (1% (w/v) TBS-T dilution) were each added in an amount of 50 μL/well to perform reaction at room temperature for 2 hours. When two types of immobilized antibodies were used, the addition amount of each immobilized antibody was halved so that the total amount was 50 μL/well. The same applied to the case in which two types of detection antibodies were used. As an antigen solution, in addition to Sample 1 of the full-length SARS-CoV-2 N protein, Sample 2, in which the total amount of the SARS-CoV-2 N protein was replaced with the equivalent mol amount of NTD and the equivalent mol amount of CTD, as a mimic of the sample in which the SARS-CoV-2 N protein was degraded, was used. After washing with TBS-T, pNPP was added in an amount of 100 μL/well as a chromogenic substrate, and the reaction was performed for 15 minutes at room temperature, after which the absorbance was measured at 405 nm using a microplate reader. Table 21 shows the results.

TABLE 21

| | | Detection | Detection | Sample 1 | Sample 2 |
|---|---|---|---|---|---|
| Immobilized | Immobilized | antibody | antibody | (0% of antigen is | (100% of antigen is |
| antibody 1 | antibody 2 | 1 | 2 | degraded.) | degraded.) |
| N1 | — | N2 | — | 0.653 | 0.245 |
| C1 | — | C2 | — | 0.292 | 0.132 |
| N1 | C1 | N2 | C2 | 0.886 | 0.320 |

As compared to the use of a single antibody as an immobilized antibody and a detection antibody, it was confirmed that use of the combination of multiple antibodies improved detection sensitivity.

### Epitope Mapping of Antibody

Epitope mapping was determined using the service of Conformational Epitope mappings produced by PEPperPRINT GmbH. In an amino acid region at positions 200 to 419 from the N-terminus of N protein of SARS-CoV-2, peptides consisting of 7, 10, and 13 amino acids were synthesized on peptide arrays so that they were shifted by 1 amino acid to overlap by 6, 9, and 12 amino acids. Based on the reactivity of antibodies, i.e., detection signals, it was identified which peptide was the epitope of antibody binding.

### Results

By epitope mapping of monoclonal antibodies, of the clones shown in Table 18, it was confirmed that three clone epitopes were in a region of SEQ ID: No. 150:LLPAADLDDFSK (at positions 394 to 405 from the N-terminus of the N protein of SARS-CoV-2), a region of SEQ ID: No. 151:KKADETQAL (at positions 374 to 382 from the N-terminus of the N protein of SARS-CoV-2), and a region of SEQ ID: No. 152:SAAEASKK (at positions 250 to 257 from the N-terminus of the N protein of SARS-CoV-2).

1. An antibody or fragment thereof specifically bindable to the amino acid sequence of SEQ ID NO: 1 or 2.

2. The antibody or fragment thereof according to claim 1, comprising
a heavy-chain CDR1 containing the amino acid sequence represented by the following formula (A-1) or (A-2):
GFX^{A31}X^{A41}X^{A51}X^{A61}X^{A71}X^{A81} (A-1)
wherein
X^{A31} represents T or S,
X^{A41} represents F, L, or I,
X^{A51} represents D, S, N, or T,
X^{A61} represents D, N, S, I, or T,
X^{A71} represents Y, F, or N,
X^{A81} represents G, W, Y, S, or T, and
GFX^{A33}X^{A43}X^{A53}X^{A63}X^{A73}X^{A83}X^{A93}X^{A103} (A-2)
wherein
X^{A33} represents T or S,
X^{A43} represents F, L, or I,
X^{A53} represents D, S, N, or T,
X^{A63} represents D, N, S, or T,
X^{A73} represents N or S,
X^{A83} represents G or Y,
X^{A93} represents Y, F, or N,
X^{A103} represents T, G, W, or Y;
a heavy-chain CDR2 containing the amino acid sequence represented by the following formula (B-1) or (B-2):
IX^{B21}X^{B31}X^{B41}X^{B51}X^{B61}X^{B71}X^{B81} (B-1)
wherein
X^{B21} represents S, N, D, W, or H,
X^{B31} represents Y, G, P, or S,
X^{B41} represents S, D, or G,
X^{B51} represents G, S, T, or A,
X^{B61} represents G, D, T, N, or R,
X^{B71} represents R, K, T, S, N, G, Y, or D,
X^{B81} represents T, I, or M, and
IX^{B23}X^{B33}X^{B43}X^{B53}X^{B63}X^{B73} (B-2)
wherein
X^{B23} represents S, N, D, W, or H,
X^{B33} represents Y, G, P, or S,
X^{B43} represents S, D, or G,
X^{B53} represents G, S, T, or A,
X^{B63} represents R, K, T, S, N, G, Y, or D,
X^{B73} represents T, I, or M;
a heavy-chain CDR3 containing the amino acid sequence represented by any one of the following formulas (C-1) to (C-8):
X^{C11}X^{C21}X^{C31}X^{C41}X^{C51}X^{C61}X^{C71}X^{C81}X^{C91}X^{C101}X^{C111}X^{C121}X^{C131}X^{C141}X^{C151} (C-1)
wherein
X^{C11} represents A or S,
X^{C21} represents R, T, or K,
X^{C31} represents D, N, S, or Y,
X^{C41} represents G, S, or Y,
X^{C51} represents G, I, or D,
X^{C61} represents S or I,
X^{C71} represents Y, V, or A,
X^{C81} represents Y, H, S, T, K, or E,
X^{C91} represents S, I, or D,
X^{C101} represents P or Y,
X^{C111} represents I, Y, G, P, T, A, or V,
X^{C121} represents N, S, Y, P, G, or F,
X^{C131} represents F, C, M, or L,
X^{C141} represents Q or D,
X^{C151} represents F, Y, V, or S,
X^{C13}X^{C23}X^{C33}X^{C43}X^{C53}X^{C63}X^{C73}X^{C83} (C-2)
wherein
X^{C13} represents A or S,
X^{C23} represents R, T, or K,
X^{C33} represents D, N, S, or Y,
X^{C43} represents I, Y, G, P, T, A, or V,
X^{C53} represents N, S, Y, P, G, or F,
X^{C63} represents F, C, M, or L,
X^{C73} represents Q or D,
X^{C83} represents F, Y, V, or S,
X^{C15}X^{C25}X^{C35}X^{C45}X^{C55}X^{C65}X^{C75}X^{C85}X^{C95}TIFTFX^{C155}X^{C165}X^{C175}X^{C185}X^{C195} (C-3)
wherein
X^{C15} represents A or S,
X^{c25} represents R, T, or K,
X^{C35} represents G, S, or Y,
X^{C45} represents G, I, or D,
C55 represents S or I,
X^{C65} represents Y, V, or A,
X^{C75} represents Y, H, S, T, K, or E,
X^{c85} represents S, I, or D,
X^{C95} represents P or Y,
X^{C155} represents I, Y, G, P, T, A, or V,
X^{C165} represents N, S, Y, P, G, or F,
X^{C175} represents F, C, M, or L,
X^{C185} represents Q or D,
X^{C195} represents F, Y, V, or S,
ARLWSGYALDI (C-4),
X^{C16}X^{C26}X^{C36}X^{C46}X^{C56}X^{C66}X^{C76}X^{C86}X^{C96}X^{C106}X^{C116}X^{C126}X^{C136} (C-5)
wherein
X^{C16} represents A or S,
X^{C26} represents R, T, or K,
X^{C36} represents D, N, S, or Y,
X^{C46} represents G, S, or Y,
X^{C56} represents G, I, or D,
X^{C66} represents Y, H, S, T, K, or E,
X^{C76} represents S, I, or D,
X^{C86} represents P or Y,
X^{C96} represents I, Y, G, P, T, A, or V,
X^{C106} represents N, S, Y, P, G, or F,
X^{C116} represents F, C, M, or L,
X^{C126} represents Q or D,
X^{C136} represents F, Y, V, or S,
VX^{C27}PLLVX^{C77}HGX^{C107}X^{C117}X^{C127}X^{C137}X^{C147} (C-6)
wherein
X^{C27} represents S or K,
X^{C77} represents V, Y, or L,
X^{C107} represents I, Y, G, P, T, A, or V,
X^{C117} represents N, S, Y, P, G, or F,
X^{C127} represents F, C, M, or L,
X^{C137} represents Q or D,
X^{C147} represents F, Y, V, or S,
VSPSX^{C58}X^{C68}X^{C78}X^{C88}X^{C98}X^{C108} (C-7)
wherein
X^{C58} represents G or A,
X^{C68} represents I, Y, G, P, T, A, or V,
X^{C78} represents N, S, Y, P, G, or F,
X^{C88} represents F, C, M, or L,
X^{C98} represents Q or D,
X^{C108} represents F, Y, V, or S, and
SPSX^{C49}X^{C59}X^{C69}X^{C79}X^{C89}X^{C99} (C-8)
wherein
X^{C49} represents G or A,
X^{C59} represents I, Y, G, P, T, A, or V,
X^{C69} represents N, S, Y, P, G, or F,
X^{C79} represents F, C, M, or L,
X^{C89} represents Q or D,
X^{C99} represents F, Y, V, or S;
a light-chain CDR1 containing the amino acid sequence represented by any one of the following formulas (D-1) to (D-3):
SX^{D21}X^{D31}X^{D41}X^{D51}X^{D61}X^{D71} (D-1)
wherein
X^{D21} represents E, D, Q, or G,
X^{D31} represents H, Y, G, or S,
X^{D41} represents R, S, I, K, or L,
X^{D51} represents S, H, N, or F,
X^{D61} represents Y or K,
X^{D71} represents Y, S, W, or N,
SX^{D23}X^{D33}X^{D43}X^{D53}X^{D63}X^{D73}GKNKDL (D-2)
wherein
X^{D23} represents E, D, Q, or G,
X^{D33} represents H, Y, G, or S,
X^{D43} represents R, S, I, K, or L,
X^{D53} represents S, H, N, or F,
X^{D63} represents Y or K,
X^{D73} represents Y, S, W, or N, and
NIGGKT (D-3);
a light-chain CDR2 containing the amino acid sequence represented by any one of the following formulas (E-1) to (E-3):
X^{E11}X^{E21}SX^{E41}GSX^{E71} (E-1)
wherein
X^{E11} represents L, V, or I,
X^{E21} represents K, N, or E,
X^{E41} represents D, Y, or K,
X^{E71} represents H or L,
VGTSDIVG (E-2), and
X^{E12}X^{E22}X^{E32} (E-3)
wherein
X^{E12} represents K, S, or W,
X^{E22} represents A or H,
X^{E32} represents N or S; and
a light-chain CDR3 containing the amino acid sequence represented by any one of the following formulas (F-1) to (F-7):
GX^{F21}X^{F31}X^{F41}X^{F51}X^{F61}X^{F71}X^{F81}X^{F91}X^{F101} (F-1)
wherein
X^{F21} represents V or A,
X^{F31} represents S, D, N, G, or K,
X^{F41} represents Y, F, V, L, or H,
X^{F51} represents S, K, or N,
X^{F61} represents G or N,
X^{F71} represents G, A, E, or K,
X^{F81} represents H, S, Y, or V,
X^{F91} represents N, G, or S,
X^{F101} represents I, V, L, or Y,
GX^{F23}X^{F33}X^{F43}X^{F53}X^{F63}X^{F73}X^{F83}X^{F93}YX^{F113} (F-2)
wherein l
X^{F23} represents V or A,
X^{F33} represents S, D, N, or G,
X^{F43} represents Y, F, V, L, or H,
X^{F53} represents S, K, or N,
X^{F63} represents G or N,
X^{F73} represents G, A, E, or K,
X^{F83} represents H, S, or Y,
X^{F93} represents N, G, or S,
X^{F113} represents I, V, L, or Y,
GX^{F24}X^{F34}X^{F44}X^{F54}IX^{F74}X^{F84}X^{F94}YX^{I114}YX^{I134} (F-3)
wherein l
X^{F24} represents V or A,
X^{F34} represents S, D, N, or G,
X^{F44} represents Y, F, V, L, or H,
X^{F54} represents S, K, or N,
X^{F74} represents G or N,
X^{F84} represents G, A, E, or K,
X^{F94} represents H, S, or Y,
X^{F114} represents N, G, or S,
X^{F134} represents I, V, L, or Y,
GX^{F25}X^{F35}X^{F45}X^{F55}X^{F65}X^{F75}X^{F85}X^{F95} (F-4)
wherein
X^{F25} represents V or A,
X^{F35} represents S, D, N, G, or K,
X^{F45} represents Y, F, V, L, or H,
X^{F55} represents S, K, or N,
X^{F65} represents G or N,
X^{F75} represents G, A, E, or K,
X^{F85} represents H, S, Y, or V,
X^{F105} represents I, V, L, or Y,
QYDSTPPLT (F-5),
QVWDSYTYV (F-6), and
QFINGPLT (F-7).

3. The antibody or fragment thereof according to claim 2, comprising
a heavy-chain CDR1 containing the amino acid sequence represented by formula (A-1),
a heavy-chain CDR2 containing the amino acid sequence represented by formula (B 1) or (B-2),
a heavy-chain CDR3 containing the amino acid sequence represented by any one of formulas (C-1), (C-2), (C-4), and (C-6) to (C-8),
a light-chain CDR1 containing the amino acid sequence represented by formula (D-1),
a light-chain CDR2 containing the amino acid sequence represented by any one of formulas (E-1) to (E-3), and
a light-chain CDR3 containing the amino acid sequence represented by any one of formulas (F-1) to (F-5).

4. The antibody or fragment thereof according to claim 2, comprising
a heavy-chain CDR1 containing the amino acid sequence represented by the following formula (A-1-1):
GFX^{A32}X^{A42}X^{A52}X^{A62}X^{A72}X^{A82} (A-1-1)
wherein
X^{A32} represents T or S,
X^{A42} represents F or L,
X^{A52} represents D, S, N, or T,
X^{A62} represents D, S, or N,
X^{A72} represents Y or F, and
X^{A82} represents G, W, S, or Y;
a heavy-chain CDR2 containing the amino acid sequence represented by the following formula (B-1-1) or (B-2-1):
IX^{B22}X^{B32}X^{B42}X^{B52}X^{B62}X^{B72}X^{B82} (B-1-1)
wherein
X^{B22} represents S, N, D, or H,
X^{B32} represents Y, G, or P,
X^{B42} represents S or D,
X^{B52} represents G or S,
X^{B62} represents G, D, T, or N,
X^{B72} represents R, K, T, S, G, or Y,
X^{B82} represents T or I, and
IWX^{B34}X^{B44}X^{B54}X^{B64}X^{B74} (B-2-1)
wherein
X^{B34} represents Y or S,
X^{B44} represents D or G,
X^{B54} represents G or A,
X^{B64} represents R or D, and
X^{B74} represents T or I,
a heavy-chain CDR3 containing the amino acid sequence represented by any one of the following formulas (C-1-1), (C-2-1), (C-6-1), (C-7-1), and (C-8-1):
X^{C12}RDGGSYX^{C82}SPIX^{C122}X^{C132}QX^{C152} (C-1-1)
wherein
X^{C12} represents A or S,
X^{C82} represents Y, H, or S,
X^{C122} represents N or S,
X^{C132} represents F or C,
X^{C152} represents F or Y, and
ATX^{C34}X^{C44}X^{C54}X^{C64}X^{C74}X^{C84} (C-2-1)
wherein
X^{C34} represents N or S,
X^{C44} represents Y or G,
X^{C54} represents N or P,
X^{C64} represents F or M,
X^{C74} represents Q or D,
X^{C84} represents Y or V,
VSPLLVVHGPFFQY (C-6-1),
VSPSGAGLDV (C-7-1), and
SPSAVGFDV (C-8-1);
a light-chain CDR1 containing the amino acid sequence represented by any one of the following formulas (D-1-1) to (D-1-7):
SEYKHYN (D-1-1),
SEHRSYY (D-1-2),
SDYSHYS (D-1-3),
SQGINKW (D-1-4),
SGHSSYY (D-1-5),
SEHSSYY (D-1-6), and
SDHSSYY (D-1-7);
a light-chain CDR2 containing the amino acid sequence represented by any one of the following formulas (E-1-1), (E-2), and (E-3-1):
X^{E12}X^{E22}SDGSX^{E72} (E-1-1)
wherein
X^{E12} represents L, V, or I,
X^{E22} represents K, or E,
X^{E72} represents H or L,
VGTSDIVG (E-2), and
KAN (E-3-1); and
a light-chain CDR3 containing the amino acid sequence represented by any one of the following formulas (F-1-1), (F-2-1), (F-3-1), (F-4-1), and (F-5):
GVSX^{F42}SGGHX^{F92}X^{F102} (F-1-1)
wherein
X^{F42} represents Y or F,
X^{F92} represents N or G,
X^{F102} represents I or V,
GADYSGASSYV (F-2-1),
GANHNIGESYGYV (F-3-1),
GVX^{F36}YX^{F56}X^{F66}X^{F76}X^{F86}X^{F96} (F-4-1)
wherein
X^{F36} represents S, G, or K,
X^{F56} represents S or N,
X^{F66} represents G or N,
X^{F76} represents G or K,
X^{F86} represents Y or V,
X^{F96} represents V or Y; and
QYDSTPPLT (F-5).

5. The antibody or fragment thereof according to claim 2, comprising
a heavy-chain CDR1 containing the amino acid sequence of any one of SEQ ID NOs: 3 to 11 or an amino acid sequence formed of any of these amino acid sequences with one to three amino acid mutations,
a heavy-chain CDR2 containing the amino acid sequence of any one of SEQ ID NOs: 12 to 25 or an amino acid sequence formed of any of these amino acid sequences with one to three amino acid mutations,
a heavy-chain CDR3 containing the amino acid sequence of any one of SEQ ID NOs: 26 to 41 or an amino acid sequence formed of any of these amino acid sequences with one to three amino acid mutations,
a light-chain CDR1 containing the amino acid sequence of any one of SEQ ID NOs: 42 to 50 or an amino acid sequence formed of any of these amino acid sequences with one to three amino acid mutations,
a light-chain CDR2 containing the amino acid sequence of any one of SEQ ID NOs: 51 to 58 or an amino acid sequence formed of any of these amino acid sequences with one to three amino acid mutations, or the amino acid sequence represented by KAN, SHN, or WAS, and
a light-chain CDR3 containing the amino acid sequence of any one of SEQ ID NOs: 59 to 73 or an amino acid sequence formed of any of these amino acid sequences with one to three amino acid mutations.

6. The antibody or fragment thereof according to claim 5, wherein the mutations are conservative substitutions.

7. The antibody or fragment thereof according to claim 2, which is specifically bindable to the amino acid sequence of SEQ ID NO: 1.

8. The antibody or fragment thereof according to claim 1, comprising
a heavy-chain CDR1 containing the amino acid sequence represented by the following formula (G-1) or (G-2):
GX^{G21}X^{G31}X^{G41}X^{G51}X^{G61}X^{G71}X^{G81} (G-1)
wherein
X^{G21} represents F or L,
X^{G31} represents I, T, S, or F,
X^{G41} represents F or L,
X^{G51} represents S, N, T, R, or D,
X^{G61} represents N, T, or D,
X^{G71} represents Y, S, or H,
X^{G81} represents F, T, Y, W, or G,
KLSERY (G-2);
a heavy-chain CDR2 containing the amino acid sequence represented by any one of the following formulas (H-1) to (H-3):
IX^{H21}X^{H31}X^{H41}X^{H51}X^{H61}X^{H71}X^{H81} (H-1)
wherein
X^{H21} represents S, G, K, or N,
X^{H31} represents G, N, S, Y, T, or P,
X^{H41} represents D, S, T, or A,
X^{H51} represents S, G, or D,
X^{H61} represents T, S, A, G, or R,
X^{H71} represents Y, K, N, T, I, or S,
X^{H81} represents I, T, P, or V,
MWSDGDT (H-2), and
NDS (H-3);
a heavy-chain CDR3 containing the amino acid sequence represented by any one of the following formulas (I-1) to (I-11):
X^{I11}X^{I21}X^{I31}X^{I41}X^{I51}X^{I61}X^{I71} (I-1)
wherein
X^{I11} represents S or T,
X^{I21} represents I or T,
X^{I31} represents N, L, D, or T,
X^{I41} represents W or G,
X^{I51} represents G, V, F, or N,
X^{I61} represents D, G, F, or N,
X^{I71} represents V or L,
ARTSRIVADLVTMGYALDF (I-2),
ARDVVKTGTTGLPFDL (I-3),
AREGVITVGTWGDV (I-4),
ATSEY (I-5),
TTATDV (I-6),
TPATDV (I-7),
SPMTIHGLDT (I-8),
GSGWV (I-9),
GSGWY (I-10), and
LSSESDDARV (I-11);
a light-chain CDR1 containing the amino acid sequence represented by any one of the following formulas (J-1) to (J-3):
X^{J11}LX^{J31}X^{J41}X^{J51}Y (J-1)
wherein
X^{J11} represents K or A,
X^{J31} represents S or P,
X^{J41} represents N, E, K, T, or D
X^{J51} represents Q, R, K, or E,
SX^{J23}SLX^{J53}X^{J63}X^{J73}DGX^{J103}TX^{J123} (J-2),
wherein
X^{J23} represents E or Q,
X^{J53} represents L or V,
X^{J63} represents H, D, or Y,
X^{J73} represents S or G,
X^{J103} represents N, K, or Y,
X^{J123} represents Y or F, and
NIGGKA (J-3),
a light-chain CDR2 containing the amino acid sequence represented by the following formula (K-1):
X^{K11}X^{K21}X^{K31} (K-1)
wherein
X^{K11} represents K, N, L, or E,
X^{K21} represents D, G, V, or I,
X^{K31} represents S, N, or D,
a light-chain CDR3 containing the amino acid sequence represented by any one of the following formulas (L-1) to (L-6):
LSX^{L31}X^{L41}X^{L51}X^{L61}X^{L71}X^{L81}X^{L91}X^{L101}X^{L111} (L-1)
wherein
X^{L31} represents R, S, A, or G,
X^{L41} represents Q, E, or D,
X^{L51} represents S or G,
X^{L61} represents D, S, or N,
X^{L71} represents D, G, or T,
X^{L81} represents A, T, or Y,
X^{L91} represents A, T, or Y,
X^{L101} represents S, R, or W,
X^{L111} represents V, L, or Y,
LSX^{L33}X^{L43}X^{L53}X^{L63}X^{L73}X^{L83}X^{L93}X^{L103} (L-2),
wherein l
X^{L33} represents R, S, A, or G,
X^{L43} represents Q, E, or D,
X^{L53} represents S or G,
X^{L63} represents D, S, or N,
X^{L73} represents D, G, or T,
X^{L83} represents A, T, or Y,
X^{L93} represents S, R, or W,
X^{L203} represents V, L, or Y,
X^{L14}QX^{L34}X^{L44}X^{L54}X^{L64}PX^{L84}X^{L94} (L-3),
wherein l
X^{L14} represents L or V,
X^{L34} represents A or T,
X^{L44} represents T or S,
X^{L54} represents H or N,
X^{L64} represents D, N, or V,
X^{L84} represents Y, V, I, or L,
X^{L94} represents T or S,
QX^{L27}X^{L37}X^{L47}X^{L57}PX^{L77}X^{L87} (L-4)
wherein
X^{L27} represents A or T,
X^{L37} represents T or S,
X^{L47} represents H or N,
X^{L57} represents D, N, or V,
X^{L77} represents Y, V, I, or L,
X^{L87} represents T or S,
QVYDSSSFV (L-5), and
WQGTDFPR (L-6).

9. The antibody or fragment thereof according to claim 8, comprising
a heavy-chain CDR1 containing the amino acid sequence represented by formula (G-1),
a heavy-chain CDR2 containing the amino acid sequence represented by formula (H 1) or (H-2),
a heavy-chain CDR3 containing the amino acid sequence represented by any one of formulas (I-1) to (I-3) and (I-5) to (I-9),
a light-chain CDR1 containing the amino acid sequence represented by any one of formulas (J-1) to (J-3),
a light-chain CDR2 containing the amino acid sequence represented by formula (K-1), and
a light-chain CDR3 containing the amino acid sequence represented by any one of formulas (L-1) to (L-3), (L-5), and (L-6).

10. The antibody or fragment thereof according to claim 8, comprising
a heavy-chain CDR1 containing the amino acid sequence represented by the following formula (G-1-1):
GX^{G22}X^{G32}X^{G42}X^{G52}X^{G62}X^{G72}X^{G82} (G-1-1)
wherein
X^{G22} represents F or L,
X^{G32} represents I, T, or S,
X^{G42} represents F or L,
X^{G52} represents S, N, T, or R,
X^{G62} represents N or T,
X^{G72} represents Y, S, or H,
X^{G82} represents F, T, Y, or W,
a heavy-chain CDR2 containing the amino acid sequence represented by the following formula (H-1-1) or (H-2):
IX^{H22}X^{H32}X^{H42}X^{H52}X^{H62}X^{F172}X^{F182} (H-1-1)
wherein
X^{H22} represents S, G, K, or N,
X^{H32} represents G, N, S, T, or P,
X^{H42} represents D, S, or A,
X^{H52} represents S, G, or D,
X^{H62} represents T, S, G, or R,
X^{H72} represents Y, K, N, T, I, or S,
X^{H82} represents I, T, P, or V, and
MWSDGDT (H-2),
a heavy-chain CDR3 containing the amino acid sequence represented by any one of the following formulas (I-1-1), (I-2), (I-3), and (I-5) to (I-9):
X^{I12}X^{I22}X^{I32}X^{I42}X^{I52}X^{I62}X^{I72} (I-1-1)
wherein
X^{I12} represents S or T,
X^{I22} represents I or T,
X^{I32} represents N, L, or T,
X^{I42} represents W or G,
X^{I52} represents G, V, F, or N,
X^{I62} represents D, F, or G,
X^{I72} represents V or L,
ARTSRIVADLVTMGYALDF (I-2),
ARDVVKTGTTGLPFDL (I-3),
ATSEY (I-5),
TTATDV (I-6),
TPATDV (I-7),
SPMTIHGLDT (I-8), and
GSGWV (I-9),
a light-chain CDR1 containing the amino acid sequence represented by any one of the following formulas (J-1-1), (J-2-1), and (J-3):
KLSX^{J42}X^{J52}Y (J-1-1)
wherein
X^{J42} represents N, E, or K,
X^{J52} represents Q, R, or E,
SX^{J24}SLX^{J54}X^{J64}SDGX^{J104}TX^{J124} (J-2-1)
wherein
X^{J24} represents E or Q,
X^{J54} represents L or V,
X^{J64} represents H, D, or Y,
X^{J104} represents N, K, or Y,
X^{J124} represents Y or F, and
NIGGKA (J-3),
a light-chain CDR2 containing the amino acid sequence represented by the following formulas (K-1-1) to (K-1-7):
NDD (K-1-1),
KDS (K-1-2),
NGN (K-1-3),
NDS (K-1-4),
KVS (K-1-5),
LIS (K-1-6), and
EVS (K-1-7), and
a light-chain CDR3 containing the amino acid sequence represented by any one of the following formulas (L-1-1), (L-2-1), (L-3-1), (L-5), and (L-6):
LSX^{L12}X^{L42}SX^{L62}DAAX^{L102}V (L-1-1)
wherein
X^{L32} represents R or S,
X^{L42} represents Q or E,
X^{L62} represents D or N,
X^{L102} represents S, R, or W,
LSGESDDAWV (L-2-1),
X^{L15}QX^{L35}THDPYT (L-3-1)
wherein
X^{L15} represents L or V,
X^{L35} represents A or T,
QVYDSSSFV (L-5), and
WQGTDFPR (L-6).

11. The antibody or fragment thereof according to claim 8, comprising
a heavy-chain CDR1 containing the amino acid sequence of any one of SEQ ID NOs: 74 to 84 or an amino acid sequence formed of any of these amino acid sequences with one to three amino acid mutations,
a heavy-chain CDR2 containing the amino acid sequence of any one of SEQ ID NOs: 85 to 101 or an amino acid sequence formed of any of these amino acid sequences with one to three amino acid mutations, or the amino acid sequence represented by NDS,
a heavy-chain CDR3 containing the amino acid sequence of any one of SEQ ID NOs: 102 to 118 or an amino acid sequence formed of any of these amino acid sequences with one to three amino acid mutations,
a light-chain CDR1 containing the amino acid sequence of any one of SEQ ID NOs: 119 to 133 or an amino acid sequence formed of any of these amino acid sequences with one to three amino acid mutations,
a light-chain CDR2 containing the amino acid sequence represented by NDD, KDS, NGN, NDS, KVS, LIS, or EVS, and
a light-chain CDR3 containing the amino acid sequence of any one of SEQ ID NOs: 134 to 149 or an amino acid sequence formed of any of these amino acid sequences with one to three amino acid mutations.

12. The antibody or fragment thereof according to claim 11, wherein the mutations are conservative substitutions.

13. The antibody or fragment thereof according to claim 8, which is specifically bindable to the amino acid sequence of SEQ ID NO: 2.

14. An antibody or fragment thereof against a nucleocapsid protein of severe acute respiratory syndrome coronavirus-2 (SARS-CoV-2), whose epitope is present in a region formed of the amino acid sequence of any of SEQ ID NOs: 150 to 152 of the nucleocapsid protein.

15. The antibody or fragment thereof according to claim 1, wherein the antibody is a monoclonal antibody.

16.-18. (canceled)

19. A kit for detecting the nucleocapsid protein of severe acute respiratory syndrome coronavirus-2 (SARS-CoV-2), comprising the antibody or fragment thereof of claim 1.

20. A kit for detecting the nucleocapsid protein of severe acute respiratory syndrome coronavirus-2 (SARS-CoV-2), comprising
two or more antibodies or fragments thereof specifically bindable to the amino acid sequence of SEQ ID NO: 1, the two or more antibodies or fragments thereof each recognizing a different epitope in the amino acid sequence of SEQ ID NO: 1, and/or
two or more antibodies or fragments thereof specifically bindable to the amino acid sequence of SEQ ID NO: 2, the two or more antibodies or fragments thereof each recognizing a different epitope in the amino acid sequence of SEQ ID NO: 2.

21. The kit according to claim 20, wherein
the two or more antibodies or fragments thereof specifically bindable to the amino acid sequence of SEQ ID NO: 1 are each the antibody or fragment thereof of any one of claims 2 to 7 or an antibody or fragment thereof containing an amino acid sequence with at least 90% identity to the amino acid sequence of the antibody or fragment thereof of any one of claims 2 to 7, and
the two or more antibodies or fragments thereof specifically bindable to the amino acid sequence of SEQ ID NO: 2 are each the antibody or fragment thereof of any one of claims 8 to 14 or an antibody or fragment thereof containing an amino acid sequence with at least 90% identity to the amino acid sequence of the antibody or fragment thereof of any one of claims 8 to 14.

22. The kit according to claim 19, for detecting the nucleocapsid protein of severe acute respiratory syndrome coronavirus-2 (SARS-CoV-2) by immunochromatography, enzyme immunoassay, chemiluminescent immunoassay, chemiluminescent enzyme immunoassay, radioimmunoassay, electrochemiluminescent immunoassay, immunonephelometry, or latex agglutination.

23. The kit according to claim 19, wherein a portion of two or more antibodies or fragments thereof specifically bindable to the amino acid sequence of SEQ ID NO: 1 or 2 is immobilized, and the remaining portion is labeled.

24. A method for detecting the nucleocapsid protein of severe acute respiratory syndrome coronavirus-2 (SARS-CoV-2), comprising the following steps (1) to (3):
(1) immobilizing a primary antibody or fragment thereof specifically bindable to the amino acid sequence of SEQ ID NO: 1 or 2 onto a solid phase,
(2) applying a secondary antibody or fragment thereof that is specifically bindable to a nucleocapsid protein of SARS-CoV-2, and that is specifically bindable to the amino acid sequence of SEQ ID NO: 1 or 2 to the solid phase, wherein the secondary antibody or fragment thereof recognizes a different epitope in an amino acid sequence to which the primary antibody or fragment thereof is specifically bindable, and the secondary antibody or fragment thereof is labeled with a labeling substance, and
(3) detecting a label derived from the labeling substance of the secondary antibody or fragment thereof bound to the solid phase via the nucleocapsid protein.

25. (canceled)

26. A kit for detecting the nucleocapsid protein of severe acute respiratory syndrome coronavirus-2 (SARS-CoV-2), comprising the antibody or fragment thereof of claim 14.


SEQUENCE LISTING (152 sequences)

SEQ ID NO: 1 - protein, 172 aa, Homo sapiens
MSDNGPQNQRNAPRITFGGPSDSTGSNQNGERSGARSKQRRPQGLPNNTASWFTALTQHGKEDLKFPRGQGVPINTNSSPDDQIGYYRRATRRIRGGDGKMKDLSPRWYFYYLGTGPEAGLPYGANKDGIIWVATEGALNTPKDHIGTRNPANNAAIVLQLPQGTTLPKGFY

SEQ ID NO: 2 - protein, 173 aa, Homo sapiens
TKKSAAEASKKPRQKRTATKAYNVTQAFGRRGPEQTQGNFGDQELIRQGTDYKHWPQIAQFAPSASAFFGMSRIGMEVTPSGTWLTYTAAIKLDDKDPNFKDQVILLNKHIDAYKTFPPTEPKKDKKKKADETQALPQRQKKQQTVTLLPAADLDDFSKQLQQSMSSADSTQA

SEQ ID NO: 3 - protein, 8 aa, Guinea pig
GFTFDDYG

SEQ ID NO: 4 - protein, 8 aa, Guinea pig
GFTFSDFW

SEQ ID NO: 5 - protein, 8 aa, Guinea pig
GFTFNNYW

SEQ ID NO: 6 - protein, 8 aa, Guinea pig
GFTFDDFG

SEQ ID NO: 7 - protein, 8 aa, Guinea pig
GFTFSNYY

SEQ ID NO: 8 - protein, 8 aa, Guinea pig
GFTFSDYW

SEQ ID NO: 9 - protein, 8 aa, Guinea pig
GFSLTSYS

SEQ ID NO: 10 - protein, 8 aa, Guinea pig
GFTFSDYT

SEQ ID NO: 11 - protein, 10 aa, Guinea pig
GFSITTNGYT

SEQ ID NO: 12 - protein, 8 aa, Guinea pig
ISYSGGRT

SEQ ID NO: 13 - protein, 8 aa, Guinea pig
INYDGDRT

SEQ ID NO: 14 - protein, 8 aa, Guinea pig
INGDSTKI

SEQ ID NO: 15 - protein, 8 aa, Guinea pig
IDYSGGTT

SEQ ID NO: 16 - protein, 8 aa, Guinea pig
ISYSGGST

SEQ ID NO: 17 - protein, 8 aa, Guinea pig
ISGDSNYI

SEQ ID NO: 18 - protein, 8 aa, Guinea pig
IHPDGGST

SEQ ID NO: 19 - protein, 8 aa, Guinea pig
ISPDGGGT

SEQ ID NO: 20 - protein, 8 aa, Guinea pig
ISPSGGST

SEQ ID NO: 21 - protein, 8 aa, Guinea pig
ISYSGGNT

SEQ ID NO: 22 - protein, 8 aa, Guinea pig
IWYDGTKM

SEQ ID NO: 23 - protein, 8 aa, Guinea pig
ISGDTNYI

SEQ ID NO: 24 - protein, 7 aa, Guinea pig
IWYDADT

SEQ ID NO: 25 - protein, 7 aa, Guinea pig
IWSGGRI

SEQ ID NO: 26 - protein, 15 aa, Guinea pig
ARDGGSYYSPINFQF

SEQ ID NO: 27 - protein, 15 aa, Guinea pig
SRDGGSYHSPISCQY

SEQ ID NO: 28 - protein, 15 aa, Guinea pig
ARDGGSYSSPINFQY

SEQ ID NO: 29 - protein, 15 aa, Guinea pig
SRDGGSYSSPINFQY

SEQ ID NO: 30 - protein, 15 aa, Guinea pig
ARDGGSVTSPIYFQV

SEQ ID NO: 31 - protein, 15 aa, Guinea pig
ARDGGSVKSPIYFQY

SEQ ID NO: 32 - protein, 8 aa, Guinea pig
ATNYNFQY

SEQ ID NO: 33 - protein, 8 aa, Guinea pig
ATSGPMDV

SEQ ID NO: 34 - protein, 19 aa, Guinea pig
ARSIIAEIPTIFTFPPFDV

SEQ ID NO: 35 - protein, 11 aa, Guinea pig
ARLWSGYALDI

SEQ ID NO: 36 - protein, 13 aa, Guinea pig
AKYYDYDYTGFDV

SEQ ID NO: 37 - protein, 14 aa, Guinea pig
VSPLLVVHGPFFQY

SEQ ID NO: 38 - protein, 14 aa, Guinea pig
VSPLLVYHGPFFQY

SEQ ID NO: 39 - protein, 14 aa, Guinea pig
VKPLLVLHGPFFQS

SEQ ID NO: 40 - protein, 10 aa, Guinea pig
VSPSGAGLDV

SEQ ID NO: 41 - protein, 9 aa, Guinea pig
SPSAVGFDV

SEQ ID NO: 42 - protein, 7 aa, Guinea pig
SEYKHYN

SEQ ID NO: 43 - protein, 7 aa, Guinea pig
SEHRSYY

SEQ ID NO: 44 - protein, 7 aa, Guinea pig
SDYSHYS

SEQ ID NO: 45 - protein, 7 aa, Guinea pig
SQGINKW

SEQ ID NO: 46 - protein, 7 aa, Guinea pig
SGHSSYY

SEQ ID NO: 47 - protein, 7 aa, Guinea pig
SEHSSYY

SEQ ID NO: 48 - protein, 7 aa, Guinea pig
SDHSSYY

SEQ ID NO: 49 - protein, 13 aa, Guinea pig
SQSLFYSGKNKDL

SEQ ID NO: 50 - protein, 6 aa, Guinea pig
NIGGKT

SEQ ID NO: 51 - protein, 7 aa, Guinea pig
LKSDGSH

SEQ ID NO: 52 - protein, 7 aa, Guinea pig
VKSDGSH

SEQ ID NO: 53 - protein, 7 aa, Guinea pig
IESDGSH

SEQ ID NO: 54 - protein, 7 aa, Guinea pig
VKSDGSL

SEQ ID NO: 55 - protein, 7 aa, Guinea pig
LNSYGSH

SEQ ID NO: 56 - protein, 7 aa, Guinea pig
LNSKGSH

SEQ ID NO: 57 - protein, 7 aa, Guinea pig
IESKGSH

SEQ ID NO: 58 - protein, 8 aa, Guinea pig
VGTSDIVG

SEQ ID NO: 59 - protein, 10 aa, Guinea pig
GVSYSGGHNI

SEQ ID NO: 60 - protein, 10 aa, Guinea pig
GVSFSGGHGV

SEQ ID NO: 61 - protein, 10 aa, Guinea pig
GVSYSGGHNV

SEQ ID NO: 62 - protein, 10 aa, Guinea pig
GVSVSGGHNL

SEQ ID NO: 63 - protein, 10 aa, Guinea pig
GVSLKGGHNL

SEQ ID NO: 64 - protein, 11 aa, Guinea pig
GADYSGASSYV

SEQ ID NO: 65 - protein, 13 aa, Guinea pig
GANHNIGESYGYV

SEQ ID NO: 66 - protein, 9 aa, Guinea pig
GVGYNNKYV

SEQ ID NO: 67 - protein, 9 aa, Guinea pig
GVGYSGGYV

SEQ ID NO: 68 - protein, 9 aa, Guinea pig
GVSYSGGYV

SEQ ID NO: 69 - protein, 9 aa, Guinea pig
GVKYSGGVV

SEQ ID NO: 70 - protein, 9 aa, Guinea pig
GVSYSGGYY

SEQ ID NO: 71 - protein, 9 aa, Guinea pig
QYDSTPPLT

SEQ ID NO: 72 - protein, 9 aa, Guinea pig
QVWDSYTYV

SEQ ID NO: 73 - protein, 8 aa, Guinea pig
QFINGPLT

SEQ ID NO: 74 - protein, 8 aa, Guinea pig
GFIFSNYF

SEQ ID NO: 75 - protein, 8 aa, Guinea pig
GFTFNTYT

SEQ ID NO: 76 - protein, 8 aa, Guinea pig
GFTFSNYY

SEQ ID NO: 77 - protein, 8 aa, Guinea pig
GLSLTNYT

SEQ ID NO: 78 - protein, 8 aa, Guinea pig
GFSFSNSW

SEQ ID NO: 79 - protein, 8 aa, Guinea pig
GFTFSNYW

SEQ ID NO: 80 - protein, 8 aa, Guinea pig
GFTFRNHW

SEQ ID NO: 81 - protein, 8 aa, Guinea pig
GFTFSNHW

SEQ ID NO: 82 - protein, 8 aa, Guinea pig
GFFFSNSW

SEQ ID NO: 83 - protein, 8 aa, Guinea pig
GFTFDDYG

SEQ ID NO: 84 - protein, 6 aa, Guinea pig
KLSERY

SEQ ID NO: 85 - protein, 8 aa, Guinea pig
ISGDSTYI

SEQ ID NO: 86 - protein, 8 aa, Guinea pig
IGNSGSKT

SEQ ID NO: 87 - protein, 8 aa, Guinea pig
ISGDGSNI

SEQ ID NO: 88 - protein, 8 aa, Guinea pig
ISGDSSNI

SEQ ID NO: 89 - protein, 8 aa, Guinea pig
IKGDSSII

SEQ ID NO: 90 - protein, 8 aa, Guinea pig
INPDDGSP

SEQ ID NO: 91 - protein, 8 aa, Guinea pig
IKGDSSIT

SEQ ID NO: 92 - protein, 8 aa, Guinea pig
INSDGSNT

SEQ ID NO: 93 - protein, 8 aa, Guinea pig
INTDGSST

SEQ ID NO: 94 - protein, 8 aa, Guinea pig
IKGDSRTI

SEQ ID NO: 95 - protein, 8 aa, Guinea pig
ISGASSNI

SEQ ID NO: 96 - protein, 8 aa, Guinea pig
ISGDSSNV

SEQ ID NO: 97 - protein, 8 aa, Guinea pig
IKGDSSTI

SEQ ID NO: 98 - protein, 8 aa, Guinea pig
INSDGSST

SEQ ID NO: 99 - protein, 8 aa, Guinea pig
IGYSGASI

SEQ ID NO: 100 - protein, 8 aa, Guinea pig
ISGTSSNI

SEQ ID NO: 101 - protein, 7 aa, Guinea pig
MWSDGDT

SEQ ID NO: 102 - protein, 7 aa, Guinea pig
TINWGDV

SEQ ID NO: 103 - protein, 7 aa, Guinea pig
STLGVGV

SEQ ID NO: 104 - protein, 7 aa, Guinea pig
TTLGVGV

SEQ ID NO: 105 - protein, 7 aa, Guinea pig
STTGFDV

SEQ ID NO: 106 - protein, 7 aa, Guinea pig
STLGNFL

SEQ ID NO: 107 - protein, 7 aa, Guinea pig
TITWGDV

SEQ ID NO: 108 - protein, 7 aa, Guinea pig
TIDWGNV

SEQ ID NO: 109 - protein, 19 aa, Guinea pig
ARTSRIVADLVTMGYALDF

SEQ ID NO: 110 - protein, 16 aa, Guinea pig
ARDVVKTGTTGLPFDL

SEQ ID NO: 111 - protein, 14 aa, Guinea pig
AREGVITVGTWGDV

SEQ ID NO: 112 - protein, 5 aa, Guinea pig
ATSEY

SEQ ID NO: 113 - protein, 6 aa, Guinea pig
TTATDV

SEQ ID NO: 114 - protein, 6 aa, Guinea pig
TPATDV

SEQ ID NO: 115 - protein, 10 aa, Guinea pig
SPMTIHGLDT

SEQ ID NO: 116 - protein, 5 aa, Guinea pig
GSGWV

SEQ ID NO: 117 - protein, 5 aa, Guinea pig
GSGWY

SEQ ID NO: 118 - protein, 10 aa, Guinea pig
LSSESDDARV

SEQ ID NO: 119 - protein, 6 aa, Guinea pig
KLSNQY

SEQ ID NO: 120 - protein, 6 aa, Guinea pig
KLSERY

SEQ ID NO: 121 - protein, 6 aa, Guinea pig
KLSKEY

SEQ ID NO: 122 - protein, 6 aa, Guinea pig
ALPKKY

SEQ ID NO: 123 - protein, 6 aa, Guinea pig
KLSTQY

SEQ ID NO: 124 - protein, 6 aa, Guinea pig
KLSDQY

SEQ ID NO: 125 - protein, 12 aa, Guinea pig
SESLLHSDGNTY

SEQ ID NO: 126 - protein, 12 aa, Guinea pig
SQSLLHSDGKTY

SEQ ID NO: 127 - protein, 12 aa, Guinea pig
SQSLVHSDGKTY

SEQ ID NO: 128 - protein, 12 aa, Guinea pig
SQSLLDSDGYTY

SEQ ID NO: 129 - protein, 12 aa, Guinea pig
SQSLLYSDGYTY

SEQ ID NO: 130 - protein, 12 aa, Guinea pig
SQSLLYSDGYTF

SEQ ID NO: 131 - protein, 12 aa, Guinea pig
SQSLLHSDGNTY

SEQ ID NO: 132 - protein, 12 aa, Guinea pig
SQSLLHGDGKTY

SEQ ID NO: 133 - protein, 6 aa, Guinea pig
NIGGKA

SEQ ID NO: 134 - protein, 11 aa, Guinea pig
LSRQSDDAASV

SEQ ID NO: 135 - protein, 11 aa, Guinea pig
LSSESDDAARV

SEQ ID NO: 136 - protein, 11 aa, Guinea pig
LSRESNDAAWV

SEQ ID NO: 137 - protein, 11 aa, Guinea pig
LSADSSGTYRV

SEQ ID NO: 138 - protein, 11 aa, Guinea pig
LSRQSDTAASL

SEQ ID NO: 139 - protein, 11 aa, Guinea pig
LSRQGDTAASY

SEQ ID NO: 140 - protein, 10 aa, Guinea pig
LSGESDDAWV

SEQ ID NO: 141 - protein, 9 aa, Guinea pig
LQATHDPYT

SEQ ID NO: 142 - protein, 9 aa, Guinea pig
VQTTHDPYT

SEQ ID NO: 143 - protein, 9 aa, Guinea pig
LQATHNPVS

SEQ ID NO: 144 - protein, 9 aa, Guinea pig
LQATHDPIT

SEQ ID NO: 145 - protein, 9 aa, Guinea pig
VQTSHVPLT

SEQ ID NO: 146 - protein, 9 aa, Guinea pig
VQTTHVPLT

SEQ ID NO: 147 - protein, 8 aa, Guinea pig
QTTNDPYT

SEQ ID NO: 148 - protein, 9 aa, Guinea pig
QVYDSSSFV

SEQ ID NO: 149 - protein, 8 aa, Guinea pig
WQGTDFPR

SEQ ID NO: 150 - protein, 12 aa, Homo sapiens
LLPAADLDDFSK

SEQ ID NO: 151 - protein, 9 aa, Homo sapiens
KKADETQAL

SEQ ID NO: 152 - protein, 8 aa, Homo sapiens
SAAEASKK